(19) United States
(12) Patent Application Publication
(10) Pub. No.: US 2024/0002358 A1
(43) Pub. Date: Jan. 4, 2024

(54) Optically clear, taste-masked, shelf-stable nano-emulsions containing cannabinoids

(71) Applicant: CCT SCIENCES, LLC, CLEARWATER, FL (US)

(72) Inventors: Harold Meckler, Delmar, NY (US); Marcus Brackeen, Research Triangle Park, NC (US); Kyle Olson, Clearwater, FL (US)

(21) Appl. No.: 18/465,928

(22) Filed: Sep. 12, 2023

Related U.S. Application Data
(63) Continuation-in-part of application No. 17/714,103, filed on Apr. 5, 2022, now Pat. No. 11,753,390.
Application No. 17/714,103 is a continuation of application No. 17/214,229, filed on Mar. 26, 2021, now Pat. No. 11,292,779.
Application No. 17/214,229 is a continuation-in-part of application No. 17/522,866, filed on Nov. 9, 2021.
(60) Provisional application No. 63/106,527, filed on Oct. 28, 2020.

(51) Int. Cl. C07D 311/80; A61K 9/00; B01D 3/10; B01D 1/22; B01D 5/00; A23L 33/105; B01D 1/08; C07B 37/10; C07B 63/02; C07B 63/04; C07B 37/08; C07B 41/06; C07B 41/04

(52) U.S. Cl. CPC C07D 311/80; A61K 9/0014; A61K 9/0056; B01D 3/10; B01D 1/22; B01D 5/0063; A23L 33/105; B01D 1/08; C07B 37/10; C07B 63/02; C07B 63/04; C07B 37/08; C07B 41/06; C07B 41/04; A23V 2002/00; C07B 2200/09; C07C 39/23

(57) ABSTRACT

The present invention describes chemical processes for making optically-clear, taste-masked, shelf-stable nano-emulsions containing essentially pure cannabinoids and other nutraceuticals.

### FIELD OF THE INVENTION

The invention relates to chemical processes for making optically-clear, taste-masked, shelf-stable nano-emulsions containing essentially pure cannabinoids and other nutraceuticals.

### BACKGROUND OF THE INVENTION

The overall bioavailability of all compounds is typically limited due to various factors of ingestion in the body. Whether the compound is hydrophilic or lipophilic, first pass metabolism and other inefficiencies can prevent over half of the nutrient from reaching the bloodstream. Typically, lipophilic compound digestion is far less efficient than hydrophilic but both can be improved with nano-based liposomal delivery systems.

The oral route of administration is the most popular vehicle for nutraceutical administration in the field today. Therefore, despite the challenges, the demand merits the development of delivery systems in this space.

Cannabinoids are an important class of diverse compounds that connect with brain receptors which are part of the endocannabinoid system. This series of receptors modulates homeostasis throughout the various facets of the human body. The most well-known receptors are the CB1 and CB2 that interact with the central nervous system and immune system respectively. There are over 100 naturally occurring cannabinoids with each affecting the body in specific ways and helping to alleviate a variety of conditions. The cannabinoid discussed in this application, Δ8-tetrahydrocannabinol, has shown great promise in promoting a general state of well-being in the user. According to The National Cancer Institute, Δ8-tetrahydrocannabinol is defined as “an analogue of Δ9-tetrahydrocannabinol with antiemetic, anxiolytic, appetite-stimulating, analgesic and neuroprotective properties.”

Δ8-tetrahydrocannabinol has a lower psychotropic potency than Δ9-tetrahydrocannabinol which may further aid in its acceptance as a medicinal based cannabinoid. Additionally, Δ8-tetrahydrocannabinol binds to both the CB1 and CB2 receptors. CB1 receptors are found in the central nervous system, mainly in the spinal cord and brain. CB2 receptors are found on cells primarily associated with the immune system and are more broadly distributed, therefore influencing most of the body. Since Δ9-tetrahydrocannabinol predominantly interacts with the CB1 receptor, it has a more limited medicinal spectrum than that of Δ8-tetrahydrocannabinol. Lastly, Δ8-tetrahydrocannabinol has shown a reduction of side effects compared to Δ9-tetrahydrocannabinol with patients reporting much less paranoia and lethargy from its use.

However, one problem is that producing cannabinoids currently requires strict federal and state licensing. Starting materials are often federally illegal with the exception of hemp grown in compliance (less than 0.3% THC) with the U.S. federal “Farm Bill of 2018”, the Agriculture Improvement Act of 2018, Pub. L. 115-334, signed into law on Dec. 20, 2018. Further, another problem is that most commercial cannabinoids vary greatly in potency and contain toxic chemicals and metals due to not being produced using state of the art pharmaceutical methods.

For preparing Δ8-tetrahydrocannabinol, Webster, Sarna, and Mechoulam (U.S. Pat. No. 7,399,872) describe a method for producing Δ8-tetrahydrocannabinol where they achieved an 81% yield of 86% Δ8-tetrahydrocannabinol as detected by High Performance Liquid Chromatography (HPLC). The problem with this method is that it produces small amounts of Δ9-tetrahydrocannabinol which means that further purification by liquid chromatography is necessary to obtain a purified Δ8-tetrahydrocannabinol product. The inventive method improves quite significantly on the overall purity and yield of the Δ8-tetrahydrocannabinol in a single step reaction. Mechoulam and Abrahamov (U.S. Pat. No. 5,605,928) also details Δ8-tetrahydrocannabinol's antiemetic effects on children. Eight children were given a dose of Δ8-tetrahydrocannabinol before chemotherapy treatment and it was shown to effectively eliminate all vomiting with little to no side effects. Δ8-tetrahydrocannabinol has been shown to be 200% more effective as an anti-emetic than Δ9-tetrahydrocannabinol and has been especially helpful when used as an anti-emetic in children. (Abrahamov et al, 1995, Life Sciences 56: 2097-2102).

The Farm Bill of 2018 gave farmers in the United States the opportunity to grow industrial hemp on a nationwide basis. Due to many of the cultivars being high in Cannabidiol, there was major interest in cultivation for biopharmaceutical applications. After harvest, industrial hemp is typically processed using solvent such as ethanol, carbon dioxide, or hydrocarbon extraction to create a full spectrum extract. Further purification occurs through distillation and isolation of the Cannabidiol to increase potency and purity of the end product. The Farm Bill of 2018 states that hemp is defined as “the plant Cannabis sativa L.” and any part of that plant, including the seeds thereof and all derivatives, extracts, cannabinoids, isomers, acids, salts, and salts of isomers, whether growing or not, with a delta-9 tetrahydrocannabinol concentration of not more than 0.3 percent on a dry weight basis.” Given the large supply of industrial hemp and Cannabidiol in the United States, there exists a need to work towards using this cannabinoid more widely to produce other medicinally beneficial compliant compounds.

Another problem involves the cost-effective commercial manufacturing of Δ9-tetrahydrocannabinol at scale.

Finally, many cannabinoids and other actives either do not mix well into oral compositions, or produce cloudy compositions. These problems affect patient compliance and/or produce unappetizing products to the consumer.

### SUMMARY OF THE INVENTION

Accordingly to address these and other problems, the invention provides an optically clear, taste-masked stable nano-emulsions containing cannabinoids and/or other nutraceuticals for oral, topical, and inhalable delivery systems. In a preferred embodiment, the nano-emulsion compositions comprise an ethanol-based phospholipid mixture, active ingredients (lipophilic or hydrophilic) including cannabinoids, terpenes, a non-ionic surfactant, and/or polymers.

Any of the nano-emulsion compositions herein may include an essentially pure Δ8-tetrahydrocannabinol oil having >99% Δ8-THC by HPLC made from using a Δ8-tetrahydrocannabinol process that does not permit isomerization of cannabidiol to Δ9-tetrahydrocannabinol during processing.

Any of the nano-emulsion compositions herein may include an essentially pure Δ9-tetrahydrocannabinol made with a process for the preparation of high purity Δ9-THC from CBD, wherein the purity is greater than 90%-95% Δ9-THC, without any significant formation of other cannabinoids

The invention includes a process for adding a signature marker molecule to authenticate the product and deter counterfeit products.

The invention also include methods of administering the nano-emulsion composition to a patient in need thereof.

### BRIEF DESCRIPTION OF THE FIGURES

FIG. 1 is an illustration of the microscopic view of a liposome particle in an aqueous solution. The labels show where various types of active ingredients will be stored in the vesicle.

FIG. 2 is an illustration of the microscopic view of a loaded invasome particle in an aqueous solution. Note how the active ingredients take their position as referenced in FIG. 1. Additionally, the invasome contains terpenes in its bilayer along with surfactants or edge activators along the outside of its shell.

FIG. 3 is an HPLC graph showing the peaks of various cannabinoids.

FIG. 4 is a Δ8-THC process flowchart showing one preferred embodiment of the inventive process described and claimed herein for obtaining a crude Δ8-THC oil having about 73.59-99.73% Δ8-THC by HPLC and less than 0.3% Δ9-THC by HPLC.

FIG. 5 is a Δ8-THC process flowchart showing another preferred embodiment of the inventive process described and claimed herein for obtaining a crude Δ8-THC oil having about 73.59-99.73% Δ8-THC by HPLC and less than Δ9-THC by HPLC and then performing vacuum distillation of the crude Δ8-THC oil with a short path vacuum distillation system to obtain a clear Δ8-THC distillate, followed by a wiped film distillation of the clear Δ8-THC distillate to obtain a Δ8-THC oil having >99% Δ8-THC by HPLC.

FIG. 6 is a Δ8-THC process flowchart showing another preferred embodiment of the inventive process described and claimed herein for obtaining a crude Δ8-THC oil having about 73.59-99.73% Δ8-THC by HPLC and less than Δ9-THC by HPLC and then performing vacuum distillation of the crude Δ8-THC oil with a short path vacuum distillation system to obtain a clear Δ8-THC distillate, followed by a wiped film distillation of the clear Δ8-THC distillate to obtain a Δ8-THC oil having >99% Δ8-THC by HPLC, followed by repeating the wiped film distillation a second time.

FIG. 7 is a Δ8-THC process flowchart showing yet another preferred embodiment of the inventive process described and claimed herein for obtaining a crude Δ8-THC oil having about 73.59-99.73% Δ8-THC by HPLC and less than Δ9-THC by HPLC, and verifying the crude Δ8-THC oil compliance of less than 0.3% Δ9-THC using post decarboxylation and/or HPLC.

FIG. 8 is a Δ8-THC process flowchart showing another preferred embodiment of the inventive process described and claimed herein for obtaining a crude Δ8-THC oil having about 73.59-99.73% Δ8-THC by HPLC and less than Δ9-THC by HPLC, verifying the crude Δ8-THC oil compliance of less than Δ9-THC using post decarboxylation and/or HPLC, then performing vacuum distillation of the crude Δ8-THC oil with a short path vacuum distillation system to obtain a clear Δ8-THC distillate, followed by a wiped film distillation of the clear Δ8-THC distillate to obtain a Δ8-THC oil having >99% Δ8-THC by HPLC, verifying the clear Δ8-THC distillate and/or Δ8-THC oil compliance of less than 0.3% Δ9-THC using post decarboxylation and/or HPLC, optionally followed by repeating the wiped film distillation a second time.

FIG. 9 is a Δ8-THC process flowchart showing another preferred embodiment of the inventive process described and claimed herein for obtaining a crude Δ8-THC oil having about 73.59-99.73% Δ8-THC by HPLC and less than Δ9-THC by HPLC, then performing vacuum distillation of the crude Δ8-THC oil with a short path vacuum distillation system to obtain a clear Δ8-THC distillate, followed by a wiped film distillation of the clear Δ8-THC distillate to obtain a Δ8-THC oil having >99% Δ8-THC by HPLC, optionally followed by repeating the wiped film distillation a second time, and verifying compliance of less than 0.3% Δ9-THC of the crude Δ8-THC oil, the clear Δ8-THC distillate and/or Δ8-THC oil using a verification method selected from the group consisting of post decarboxylation, HPLC, gas chromatography (GC), GC coupled with mass spectrometry (MS), GC coupled with flame ionization detection (FID), HPLC with MS, HPLC with ultraviolet (UV) absorbance, HPLC with diode array detection (DAD), HPLC-electrospray ionization-quadrupole time of flight (ESI-qT0F), HPLC-MS/MS, ultra-performance liquid chromatography (UPLC), UPLC-qT0F, matrix assisted laser desorption ionization mass spectrometry (MALDI-MS), thin layer chromatography (TLC), Fourier transform infrared spectroscopy (FTIR), and nuclear magnetic resonance spectrometry (NMR).

FIG. 10 is a Δ9-THC process flowchart showing one preferred embodiment of the inventive process described and claimed herein for (i) extracting a cannabidiol extract from industrial hemp having less than 0.3% Δ9-THC, dissolving the extract in a solvent, reacting with an organoaluminum catalyst in an inert atmosphere, quenching and filtering the reaction mixture; (ii) performing short path vacuum distillation followed by wiped film distillation to obtain a Δ9-THC oil comprising about 93% Δ9-THC and about 4% unreacted CBD by HPLC.

FIG. 11 is a Δ9-THC process flowchart showing another preferred embodiment of the inventive process described and claimed herein for (i) extracting a cannabidiol extract from industrial hemp having less than 0.3% Δ9-THC, dissolving the extract in a solvent, and reacting with an organoaluminum catalyst in hexane in an inert atmosphere under ambient temperature, (ii) stirring the for 6-20 hours at −20° C. to 70° C., quenching with water or alcohol, and filtering and rinsing the reaction mixture using dichloromethane, hexanes, or a combination; (iii) Vacuum distilling the crude Δ9-THC oil with a short path vacuum distillation system at 15-20 mTorr until a clear Δ9-THC distillate starts to condense and then immediately stopping the vacuum distilling, wherein said vacuum distilling removes residual solvent and volatile cannabidiol impurities (low boilers) from the clear Δ9-THC distillate; and (iv) Wiped film distilling the clear Δ9-THC distillate with a wiped film distillation unit to obtain a Δ9-THC oil having >90 or 98-99% Δ9-THC by HPLC, wherein said wiped film distilling separates the desired product from the high temperature cannabinoid impurities having a non-vacuum boiling higher than 180° C.

FIG. 12 is a Δ9-THC process flowchart showing another preferred embodiment of the inventive process described and claimed herein for (i) extracting a cannabidiol extract from industrial hemp having less than 0.3% Δ9-THC, dissolving the extract in a solvent, and reacting with an organoaluminum catalyst in hexane in an inert atmosphere under ambient temperature; (ii) stirring the for 6-20 hours at −20° C. to 70° C., quenching with water or alcohol, and filtering and rinsing the reaction mixture using dichloromethane, hexanes, or a combination; (iii) Vacuum distilling the crude Δ9-THC oil with a short path vacuum distillation system at 15-20 mTorr until a clear Δ9-THC distillate starts to condense and then immediately stopping the vacuum distilling, wherein said vacuum distilling removes residual solvent and volatile cannabidiol impurities from the clear Δ9-THC distillate; and (iv) Wiped film distilling the clear Δ9-THC distillate with a wiped film distillation unit to obtain a Δ9-THC oil having >90-99% Δ9-THC by HPLC, wherein said wiped film distilling removes high temperature cannabinoid impurities having a non-vacuum boiling higher than 180° C.; (v) performing step (iv) a second time.

FIG. 13 is a Δ9-THC process flowchart showing another preferred embodiment of the inventive process described and claimed herein for (i) verifying a source of industrial hemp as having <0.3% Δ9-THC by HPLC; (ii) extracting a cannabidiol extract from industrial hemp having less than 0.3% Δ9-THC, dissolving the extract in a solvent, and reacting with an organoaluminum catalyst in hexane in an inert atmosphere under ambient temperature, (iii) stirring the for 6-20 hours at −20° C. to 70° C., quenching with water or alcohol, and filtering and rinsing the reaction mixture using dichloromethane, hexanes, or a combination; (iv) Vacuum distilling at 15-20 mTorr the crude Δ9-THC oil with a short path vacuum distillation system until a clear Δ9-THC distillate starts to condense and then immediately stopping the vacuum distilling, wherein said vacuum distilling removes residual solvent and volatile cannabidiol impurities from the clear Δ9-THC distillate; and (v) Wiped film distilling the clear Δ9-THC distillate with a wiped film distillation unit to obtain a Δ9-THC oil having >90-99% Δ9-THC by HPLC, wherein said wiped film distilling removes high temperature cannabinoid impurities having a non-vacuum boiling higher than 180° C.

FIG. 14 is a Δ9-THC process flowchart showing another preferred embodiment of the inventive process described and claimed herein for: (i) verifying a source of industrial hemp as having <0.3% Δ9-THC by HPLC; (ii) extracting a cannabidiol extract from industrial hemp having less than 0.3% Δ9-THC, dissolving the extract in a solvent, and reacting with an organoaluminum catalyst in hexane in an inert atmosphere under ambient temperature; (iii) stirring the for 6-20 hours at −20° C. to 70° C., quenching with water or alcohol, and filtering and rinsing the reaction mixture using dichloromethane, hexanes, or a combination; (iv) Vacuum distilling at 15-20 mTorr the crude Δ9-THC oil with a short path vacuum distillation system until a clear Δ9-THC distillate starts to condense and then immediately stopping the vacuum distilling, wherein said vacuum distilling removes residual solvent and volatile cannabidiol impurities from the clear Δ9-THC distillate; and (v) Wiped film distilling the clear Δ9-THC distillate with a wiped film distillation unit to obtain a Δ9-THC oil having >90-99% Δ9-THC by HPLC, wherein said wiped film distilling removes high temperature cannabinoid impurities having a non-vacuum boiling higher than 180° C.; (vi) verifying purity of >90-99% Δ9-THC using a verification method selected from the group consisting of post decarboxylation, HPLC, gas chromatography (GC), GC coupled with mass spectrometry (MS), GC coupled with flame ionization detection (FID), HPLC with MS, HPLC with ultraviolet (UV) absorbance, HPLC with diode array detection (DAD), ultra-performance liquid chromatography (UHPLC), thin layer chromatography (TLC), Fourier transform infrared spectroscopy (FTIR), and nuclear magnetic resonance spectrometry (NMR), UV Spectroscopy.

FIG. 15 is a Δ9-THC flow chart showing another preferred embodiment of the inventive process described and claimed herein for adding a signature marker molecule to a product containing the Δ9-THC oil having >90-99% Δ9-THC made by the process herein.

FIG. 16 is a bar graph of a prospective example showing the use of a signature marker to authenticate the high purity Δ9-tetrahydrocannabinol made according to the process herein. FIG. 8 shows an example of a rt-PCR graph, where the resulting cycle threshold (Cq) can be compared against negative and positive controls, as shown.

### DETAILED DESCRIPTION OF THE INVENTION

In a preferred embodiment, the invention relates to optically clear, taste-masked stable nano-emulsions containing cannabinoids and/or other nutraceuticals for oral, topical, and inhalable delivery systems. In a preferred embodiment, the nano-emulsion compositions comprise an ethanol-based phospholipid mixture, active ingredients (lipophilic or hydrophilic) including cannabinoids, terpenes, and a surfactant. The surfactant can also function as an edge activator that modulates onset time and location. The resulting nano-emulsion composition is then either introduced into an aqueous solution for activation or is presented in some other delivery system.

By using terpenes, the nano-emulsion composition shows greater bioavailability of the vesicles over traditional liposomes, ethosomes, transfersomes, and niosomes. What has been shown is a synergistic effect of the terpenes and solvents to allow the body to absorb the compounds at a much higher efficiency.

The invention further discloses a method of marking a nano-emulsion composition that forms optically clear nano-emulsions. Due to the proper ratio of phospholipids, solvent, and surfactant the invention provides a transparent activated solution that is also shelf stable.

Another novel aspect of this invention is that the nano particulate concentrate is tasteless in nature. Various natural extracts are used to balance out any negative taste components of the active ingredients. This offers a one-step infusion to the end delivery system without having to use any additional masking agents in the production process.

Finally, the present invention has been formulated to allow for greater stability of lipophilic compounds in a final aqueous delivery system. By using non-ionic surfactants and/or polymers the invention has been able to provide stabilized solutions for extended shelf life in physical appearance and in particle size distribution.

Any of the nano-emulsion compositions herein may include an essentially pure Δ8-tetrahydrocannabinol made from using a process that does not permit isomerization of cannabidiol to Δ9-tetrahydrocannabinol during processing to maintain compliance with federal laws throughout processing, and using a differential distillation using vacuum distillation for volatile or low temperature impurities and wipe film distillation to remove high temperature impurities.

Any of the nano-emulsion compositions herein may include Δ8-tetrahydrocannabinol made with a process that starts with an industrial hemp plant that is less than 0.3% Δ9-THC. The cannabidiol (CBD) extract obtained from the compliant (verified less than 0.3% Δ9-THC) hemp is processed to also have less than 0.3% Δ9-THC. The next step of processing with an organic acid, e.g. p-toluenesulfonic acid, in a chemically-related and compatible solvent, e.g. toluene, followed by quenching with a weak base, e.g. sodium bicarbonate, and washing with water, also yields a crude Δ8-THC oil having less than 0.3% Δ9-THC. Thus, the entire Δ8-THC process stays Δ9-THC-compliant at each step. Further performing a short-path vacuum distillation to remove the low temperature impurities ensures that the crude Δ8-THC oil produces a Δ8-THC distillate without allowing cannabidiol to isomerize to the unwanted and non-compliant Δ9-THC. Lastly, performing a wiped film distillation to remove the high temperature impurities also ensures that the Δ8-THC distillate produces a highly pure Δ8-THC oil having >99% Δ8-THC by HPLC without allowing any further isomerization to the unwanted and non-compliant Δ9-THC.

The invention also includes compositions and formulations containing the Δ8-THC oil having >99% Δ8-THC by HPLC.

Any of the nano-emulsion compositions herein may include an essentially pure Δ9-tetrahydrocannabinol made with a process for the preparation of high purity Δ9-THC from CBD, wherein the purity is greater than 90%-95% Δ9-THC, without any significant formation of other cannabinoids, including without Δ8-THC or the other isomers of THC. The only measurable cannabinoid in this invention, other than the desired Δ9-THC, consists of unreacted CBD.

Any of the nano-emulsion compositions herein that include Δ9-tetrahydrocannabinol may be made with a process that starts an industrial hemp plant that is less than 0.3% Δ9-THC and using extraction and purification techniques to derive a cannabidiol (CBD) distillate or isolate. The next steps comprise: dissolving the CBD distillate or isolate in dichloromethane to create a homogenized mixture; adding the homogenized mixture to a reactor vessel continuously purged with an inert gas and adding a 10 mol % solution of organoaluminum catalyst in hexane slowly over 30 minutes at a temperature of 18-26° C. to create a reaction mixture; stirring the reaction mixture for approximately 6-20 hours at a temperature of −20° C. to about 70° C.; quenching the reaction mixture with water or a C2-C4 alcohol, and stirring for 1 hour; filtering the reaction mixture through a filter of diatomaceous earth, perlite, or cellulose to collect a filtrate, and rinsing the filter and reaction vessel with a rinse solvent selected from dichloromethane, hexanes, or a combination of both, and combining the filtrate and the rinse solvent to obtain a combined filtrate and rinse; and performing a split path distillation of the combined filtrate and rinse, wherein the split path distillation comprises a short path distillation and a wiped film distillation to remove terpenes, high volatiles, or high boiling point cannabinoids from the combined filtrate and rinse, to obtain a Δ9-THC oil comprising about 93% or greater Δ9-THC and trace amounts of about 4% CBD.

The invention includes a process for adding a signature marker molecule to authenticate the product and deter counterfeit products.

The invention also includes a method of administering the nano-emulsion composition containing the Δ9-THC oil provided by the process herein to a patient in need thereof, comprising formulating the Δ9-THC oil as an oral or topical composition, and delivering the oral or topical composition to a patient in need thereof, wherein the patient has nausea, anxiety, stress, chronic pain, acute pain, opioid withdrawal, narcotic relapse risk, or requires an appetite stimulant.

### PREFERRED EMBODIMENTS—NANOEMULSIONS

The present invention is directed to nano-particulate compositions comprising nutraceutical particles having an effective particle size of less than 120 nm. This threshold is commonly accepted as the point at which optical clarity is shown in aqueous solutions. Some of the most popular nutraceuticals tested include: Vitamins & Minerals, Herbs, Electrolytes, Cannabinoids, Caffeine, Health Supplements, and Sports Supplements.

The present invention provides formulations and methods for improving the oral, topical, and inhalable bioavailability of nutraceutical compositions.

Liposomes may be used as a vehicle for encapsulating compounds that are either hydrophilic or lipophilic in nature. The lipid bilayer allows for the lipophilic ingredients to stabilize while the aqueous core invites hydrophilic actives to be included. Through these vesicles both types of active ingredients can be reduced in particle size, protected, and made more bioavailable to the system. This, in turn, will create better absorption, quicker onset, and greater efficacy of all loaded components. Once all the contents have been mixed, the liposomal solution is added to an aqueous solution which causes spontaneous self-emulsification of nanoparticles. This is commonly referred to in the pharmaceutical arts as a Self Nano-Emulsifying Drug Delivery System (SNEDDS). SNEDDS are able to form nano particles of less than 200 nm. This feature is very beneficial as for some use cases, these particles are small enough and don't require any further processing or specialized equipment to reach its full potential.

For oral applications particularly, some of the shortcomings of the current technology revolves around three factors: optical clarity, taste, and stability. Many liposomal formulations have inefficiencies in one of these core issues that limits its commercialization potential. The cannabinoid industry is one that is particularly sensitive to these very problems. Many of the liposomal products on the market form cloudy aqueous solutions with unappetizing taste profiles. Additionally, many of these emulsions have a hard time holding the cannabinoids in solution for any length of time.

For topical applications, many of the previous liposomal technologies focus on penetration enhancement. While the traditional liposome is a satisfactory vehicle for compound delivery, it is not the best for skin penetration. Typically, solvents such as ethanol are used to loosen the turbidity of the cell wall in the liposome and allow for it to be more malleable as it traverses the layers of the dermis. Transcutal has also been used quite often in the prior art to accomplish this task without the use of ethanol.

In the present invention, terpenes are used to increase the bioavailability in oral and topical applications. The terpenes work synergistically with the solvent(s) to establish a vesicle that is more receptive to the body. Terpenes work as a permeation enhancer that fluidizes the intercellular lipids which stimulates absorption. Even at low doses (0.25-5%), select terpenes have the ability to increase all the benefits associated with liposomal encapsulation.

In the present invention, a blend of ingredients is used to achieve the formulation and meet required parameters for optical clarity, taste profile, and stability. A specific blend or composition is required since not every combination of surface stabilizer and active agent will result in a stable nano-particulate composition. Additionally, the specific blend or combination of ingredients is required to be formulated using specialized processing techniques to arrive at a product that meets the criteria listed above.

For example, issues of optical clarity, taste profile, and stability are not met when making a vesicle called a transethosome which combines an ethanol based solution of lecithin or glyceride composition with an active ingredient and a non-ionic surfactant. This method may work to achieve nano particles of the right size, ranging from 150-1000 nm in size, but does not work to achieve optical clarity, taste profile, and stability. Many of these aqueous activated emulsions are cloudy, milky, or have a blue-ish appearance. Further, these emulsions have harsh taste profiles with chemical or bitter notes from the active ingredient and the non-ionic surfactant.

With much competition in the retail market for nutraceuticals optical clarity and an enjoyable taste profile are essential to gaining acceptance on a commercial basis.

According to the present invention, introducing terpenes into the lipid bilayer increases the absorbability to levels above prior liposome based delivery systems. This includes but is not limited to classic liposomes, ethosomes, transethosomes, transferosomes, niosomes, and solid lipid nanoparticles.

Further, incorporation of specialized techniques achieves the optical clarity and stability required in the present invention, but not possible with aqueous solutions.

The present invention may also use one or more infusions of specific extracts in the inventive nano-particulate solution to completely mute any off-tasting attributes of the active ingredient or surfactant used.

The formulation can then be further processed to reduce particle size further, gain increased uniformity in the particle distribution, or to change the state of the ingredients for various delivery systems. To further reduce particle size, a High Pressure Homogenizer can be used. High Pressure Homogenization is a technique that pushes a solution through a channel at very high pressures (25,000 PSI) causing cell lysis and therefore reducing the size of the vesicles.

Careful selection had to be made on how to formulate our nano particulate solution. A modified version of the hot method common in literature was shown to be the preferred method of processing due to its ability to allow more fluidity in the input materials. This was shown to allow more complete encapsulation and better clarity in our work.

Absolute Ethanol was heated to the proper temperature and the lipid was added along with the active ingredients. Continuing to heat the solution, the terpenes and non-ionic surfactant were then mixed into the vessel. Lastly, while still under heat, the natural extracts for taste and glycerine were added to promote optical clarity. This solution is then stirred for an additional period of time to reach optimal homogeneity.

Further processing can be done for specific delivery systems that have limitations or protocols that must be used to garner market traction.

In some embodiments, the solution discussed above is satisfactory.

### Independent Claims

In a preferred non-limiting embodiment, the invention provides a process of making a self-emulsifying nano-concentrate for encapsulating a lipophilic active ingredient, comprising the steps: dissolving 95% phosphatidylcholine into 60° C. ethanol; heating the lipophilic active ingredient to 60° C. and adding it to the hot ethanol/PC solution; stirring until homogenized; dissolving Poloxamer 407 to the solution; stirring glycerin, d-limonene terpenes, cherry tart extract, and a natural flavor blend into the solution; and adding to an aqueous solution to obtain the self-emulsifying nano-concentrate having the encapsulated lipophilic active ingredient, wherein the self-emulsifying nano-concentrate is optically clear (transparent), has no lingering taste, and is shelf stable.

In a preferred non-limiting embodiment, the invention provides a process of making a self-emulsifying nano-concentrate for encapsulating a hydrophilic ingredient, comprising the steps: dissolving phosphatidylcholine into 60° C. ethanol; separately combining reverse osmosis water and a hydrophilic active ingredient to form an aqueous solution; adding the ethanol/PC solution to the aqueous solution to obtain the self-emulsifying nano-concentrate having the encapsulated hydrophilic active ingredient, wherein the self-emulsifying nano-concentrate is optically clear (transparent), has no lingering taste, and is shelf stable.

In a preferred non-limiting embodiment, the invention provides a process of making a combined concentrate for encapsulating an active lipophilic ingredient and an active hydrophilic ingredient, comprising the steps: providing the self-emulsifying nano-concentrate having a lipophilic active ingredient that was made in claim 1 and heating to 60° C.; separately combining reverse osmosis water and a hydrophilic active ingredient to form an aqueous solution; adding the self-emulsifying nano-concentrate having a lipophilic active ingredient to the aqueous solution having the hydrophilic active ingredient to obtain a combined nano-concentrate having the lipophilic active ingredient and the hydrophilic active ingredient, wherein the combined nano-concentrate is optically clear (transparent), has no lingering taste, and is shelf stable.

In a preferred non-limiting embodiment, the invention provides a process of making an ethanol-free oral formulation for encapsulating a lipophilic active ingredient, comprising the steps: providing the self-emulsifying nano-concentrate having the lipophilic active ingredient that was made in claim 1 and heating to 60° C.; loading the heated self-emulsifying nano-concentrate into a rotary evaporator; raising the temperature to 80° C. to condense and collect the ethanol, and optionally, introducing vacuum at 700 micron until no more ethanol is visible on the condenser; cooling to room temperature; pulling the vacuum down to 500 micron and holding the vacuum overnight to ensure all ethanol is removed and to obtain a film; heating the film back to 50° C.; introducing reverse osmosis water to the film and spinning on a rotary evaporator to produce invasomes as the ethanol-free oral formulation.

In a preferred non-limiting embodiment, the invention provides a process of making an ethanol-free topical formulation for encapsulating a lipophilic active ingredient, comprising the steps: dissolving 95% phosphatidylcholine into ethyl lactate at 60° C.; heating the lipophilic active ingredient to 60° C. and adding to the hot ethyl lactate/PC solution; adding PEG-40 Hydrogenated Castor Oil to the ethyl lactate/PC solution; adding d-limonene terpenes to the ethyl lactate/PC/PEG solution to obtain the ethanol free topical formulation having a lipophilic active ingredient.

In a preferred non-limiting embodiment, the invention provides a process of making a natural topical formulation for encapsulating a lipophilic active ingredient, comprising the steps: dissolving phosphatidylcholine into 60° C. ethanol; heating the lipophilic active ingredient to 60° C. and adding to the hot ethanol/PC solution; dissolving decyl glucoside (DG) into the ethanol/PC solution; adding a solution of d-limonene terpenes to the ethanol/PC/DG solution to obtain the natural topical formulation having a lipophilic active ingredient.

In a preferred non-limiting embodiment, the invention provides a process of making a glyceride formulation for encapsulating a lipophilic active ingredient, comprising the steps: dissolving Capmul MCM mono & di-glyceride emulsifier and Captex 355 caprylic/capric triglyceride (glyceride) into ethanol at 60° C.; heating the lipophilic ingredient to 60° C. and adding to the hot ethanol/glyceride solution; adding Poloxamer 407 to the ethanol/glyceride solution; adding glycerin, d-limonene terpenes, cherry tart extract, and natural flavor blend to obtain the glyceride formulation having the lipophilic active ingredient.

In a preferred non-limiting embodiment, the invention provides a process of making a sub-100 nm invasome formulation for encapsulating a lipophilic active ingredient, comprising the steps: providing the self-emulsifying nano-concentrate having the lipophilic active ingredient that was made in claim 1 and heating to C; adding reverse osmosis water to the self-emulsifying nano-concentrate on a spinning rotary evaporator to produce invasomes; loading the invasomes into a high pressure homogenizer; in one hour (4 cycles) applying 15,000 PSI to the solution; Once complete, changing the pressure to 25,000 PSI and running for an additional 2 hours (4 cycles), to obtain the sub-100 nm invasome formulation having the lipophilic active ingredient.

In a preferred non-limiting embodiment, the invention provides a process of making a lyophilized ingestible/topical freeze dried powder, comprising the steps: providing the self-emulsifying nano-concentrate having the lipophilic active ingredient that was made in claim 1 and heating to 60° C.; adding Bovine Serum Albumin (BSA) to the self-emulsifying nano-concentrate; freezing the self-emulsifying nano-concentrate/BSA solution at −25° C. overnight; loading the frozen self-emulsifying nano-concentrate/BSA solution into a freeze dryer and running through at −20° C. for 12 hours at 350 microns of vacuum, to obtain the finished lyophilized ingestible/topical freeze dried powder having the lipophilic active ingredient, wherein the finished powder has a grainy consistency and is re-hydrated into nanoparticles by adding at least equal parts finished powder to reverse osmosis water.

In a preferred non-limiting embodiment, the invention provides a process of making a lyophilization inhalable freeze dried powder for encapsulating a lipophilic active ingredient, comprising the steps: dissolving 95% phosphatidylcholine into ethanol at 60° C.; heating the lipophilic active ingredient to 60° C. and adding to the hot ethanol/PC solution; dissolving PEG-40 Hydrogenated Castor Oil into the ethanol/PC solution; adding d-limonene terpenes and mannitol to the ethanol/PC/PEG solution; freezing the ethanol/PC/PEG solution at −25° C. overnight; loading the frozen solution into a freeze dryer and running through at −20° C. for 12 hours at 350 microns of vacuum to obtain a finished powder having the lipophilic active ingredient, wherein the finished powder has a grainy consistency and wherein the finished powder is loaded into a metered dose inhaler, dry powder inhaler, or a nebulizer for delivery.

Any of the preferred non-limiting embodiments may include wherein the lipophilic active ingredient is selected from the group consisting of: Δ8-tetrahydrocannabinol, Δ9-tetrahydrocannabinol (THC), cannabidiol (CBD), CBD distillate, CBD isolate, cannabinol (CBN), cannabigerol (CBG), Δ9(11)-tetrahydrocannabinol (exo-THC), cannabichromene (CBC), tetrahydrocannabinol-C3 (THC-C3), tetrahydrocannabutol (THC-C4), and mixtures thereof.

Any of the preferred non-limiting embodiments may include wherein the lipophilic active ingredient is selected from the group consisting of: >99% pure Δ8-tetrahydrocannabinol (THC) oil having less than 0.3% Δ9-THC; Δ9-tetrahydrocannabinol (THC) oil comprising over about 90% Δ9-THC, cannabidiol (CBD), CBD distillate, CBD isolate, and mixtures thereof.

Any of the preferred non-limiting embodiments may include wherein the invention is a composition having a lipophilic active ingredient made using the lipophilic processes herein.

Any of the preferred non-limiting embodiments may include wherein the hydrophilic active ingredient is a hydrophilic prodrug of a cannabinoid, the hydrophilic prodrug group selected from the group consisting of hemisuccinate, valine hemisuccinate, organophosphate ester (0-phosphate), acetate ester (0-acetate), and morpholinylbutyrate, and wherein the cannabinoid is selected from from the group consisting of: Δ8-tetrahydrocannabinol, Δ9-tetrahydrocannabinol (THC), cannabidiol (CBD), CBD distillate, CBD isolate, cannabinol (CBN), cannabigerol (CBG), Δ9(11)-tetrahydrocannabinol (exo-THC), cannabichromene (CBC), tetrahydrocannabinol-C3 (THC-C3), tetrahydrocannabutol (THC-C4), and mixtures thereof.

Any of the preferred non-limiting embodiments may include wherein the invention is a composition having a hydrophilic active ingredient made using the hydrophilic processes herein.

### Dependent Claims

Any of the preferred non-limiting embodiments may include wherein the compositions are loaded into a capsule or beadlet for product delivery. Upon the system dissolving in the stomach, the solution will self nano-emulsify in the gastric fluid.

Any of the embodiments herein may include wherein the solution discussed above is used ‘as is’ by loading it into a delivery system such as a beverage, edible, or gummy production line. As the solution is mixed with the aqueous ingredients, it will self nano-emulsify and be ready for ingestion.

Any of the embodiments herein may include wherein the solution discussed above is further processed by rotary evaporator to remove all traces of the ethanol and make a film of the remaining components. This is typically referred to as the Thin-Film Hydration Method and is common in the field. Upon rehydration with an aqueous solution, the film instantaneously forms an optically clear, tasteless, nano emulsion.

Any of the embodiments herein may include wherein the solution discussed above is added to a cryoprotectant and the solution is then loaded into a freeze-spray drying machine for lyophilization. This will create a solid crystalline powder that can be rehydrated to spontaneously form an optically clear, tasteless, nano emulsion.

Any of the embodiments herein may include wherein the solution discussed above is added to a specified amount of aqueous solution and further processed by high pressure homogenization. This creates particles that are sub-100 nm in size which still have all the same clarity and taste benefits discussed in other embodiments.

Acceptable solvents include ethanol, heptane, hexane, DMSO, transcutol, ethyl acetate, ethyl lactate.

Acceptable co-solvents include glycerin.

Acceptable lipids sources include but are not limited to lecithin based phospholipids such as phosphatidylcholine (PC), lysophosphatidylcholine (LPC), phosphatidylinositol (PI), phosphatidylethanolamine (PE), phosphatidylserine (PS), and phosphatidic acid (PA). Lipid sources can also include mono, di, and triglycerides. Purified concentrates of these lipids are readily available on the commercial market.

Acceptable terpenes include but are not limited to alpha bisabolol, alpha cedrene, alpha phellandrene, alpha pinene, alpha terpineol, beta caryophyllene, beta pinene, borneol, cadinene, camphene, camphor, citral, citronellol, delta 3 came, d-limonene, eucalyptol, eugenol, farnesene, fenchol, gamma terpinene, geraniol. geranyl acetate, humulene, limonene, linalool, menthol, myrcene, nerol, nerolidol, ocinene, para-cymene, phytol, terpineol, terpinolene, valencene.

Acceptable surfactants include Polyethylene glycol and derivatives, Polysorbates, Poloxamers, HPC, HPMC, PLGA, PLA, PVA, PVA-PEG, PVP, Sodium Lauryl Sulfate, Vitamin E TPGS, Coco Glucoside, Decyl Glucoside.

Acceptable cryoprotectants include bovine serum albumin, mannitol, sorbitol, lactose, maltose, PEG3350, PVP K15, PVP K30, glucose, trehalose.

### Figures—Nano-Emulsion

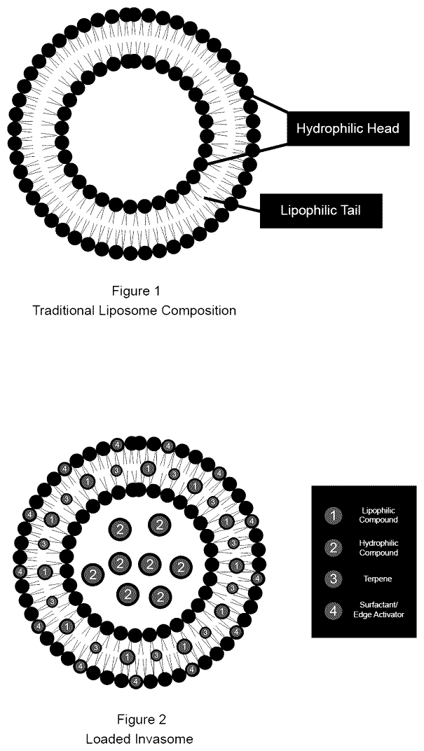

Referring now to the figures, FIG. 1 illustrates the microscopic view of a traditional liposome particle in an aqueous solution. The labels show where various types of active ingredients will be stored in the vesicle.

FIG. 2 illustrates the microscopic view of a loaded invasome particle in an aqueous solution. Note how the active ingredients take their position as referenced in FIG. 1. Additionally, the invasome contains terpenes in its bilayer along with surfactants or edge activators along the outside of its shell.

### EXAMPLES—NANO-EMULSION

### Example—Making the Self Nano Emulsifying Concentrate (For Lipophilic Ingredients)

630 g of absolute ethanol is added to a beaker on a heated stir plate. The ethanol is continuously stirred while being warmed to 60° C. Once at 60° C., 10 g of 95% phosphatidylcholine is dissolved into the ethanol. Next, 150 g of a lipophilic ingredient is heated in an oven to 60° C. and added to the hot ethanol/PC solution. After it is homogenized, 150 g of Poloxamer 407 is added to the solution. Through stirring and heat, the Poloxamer will disperse completely into the ethanol. After complete dissolution, a solution of 25 g glycerin, 10 g of d-limonene terpenes, 15 g cherry tart extract, and 10 g of a proprietary natural flavor blend are added to the main ethanol beaker. Allow to stir for an additional 20 minutes at temperature.

When added to an aqueous solution, this concentrate is optically clear (transparent), has no lingering taste, and is shelf stable.

### Example—Making the Self Nano Emulsifying Concentrate (For Lipophilic Ingredients) Failed Example

630 g of absolute ethanol is added to a beaker on a heated stir plate. The ethanol is continuously stirred while being warmed to 60° C. Once at 60° C., 10 g of 95% phosphatidylcholine is dissolved into the ethanol. Next, 150 g of a lipophilic ingredient is heated in an oven to 60° C. and added to the hot ethanol/PC solution. After it is homogenized, 200 g of PEG-40 Hydrogenated Castor Oil is added to the solution. Through stirring and heat, the PEG will disperse completely into the ethanol. Allow to stir for an additional 20 minutes at temperature.

When added to an aqueous solution, this concentrate is blueish in color, has a very bitter/chemical lingering taste, and is shelf stable.

### Example—Making the Self Nano Emulsifying Concentrate (For Hydrophilic Ingredients)

95 g of absolute ethanol is added to a beaker on a heated stir plate. The ethanol is continuously stirred while being warmed to 60° C. Once at 60° C., 5 g of 95% phosphatidylcholine is dissolved into the ethanol.

In a separate beaker add 800 g of reverse osmosis water and 100 g of a hydrophilic active ingredient and stir until completely homogenized. Once complete take the ethanol blend and add it to the aqueous solution while stirring. Allow to stir for an additional 20 minutes.

When added to an aqueous solution, this concentrate is optically clear (transparent), has no lingering taste, and is shelf stable.

### Example—Combinative Technique (Encapsulate Lipophilic and Hydrophilic Ingredients)

Take the solution that was formulated in Example 1 and pour it into a beaker on a heated stirplate. Bring the temperature up to 60° C. and continue to stir.

In a separate beaker add 900 g of reverse osmosis water and 100 g of a hydrophilic active ingredient and stir until completely homogenized. Once complete take the ethanol blend and add it to the aqueous solution while stirring. Allow to stir for an additional 20 minutes.

When added to an aqueous solution, this concentrate is optically clear (transparent), has no lingering taste, and is shelf stable.

### Example—Removing the Ethanol (Ethanol Free Oral Formulation)

Take the solution that was formulated in Example 1 and pour it into a beaker on a heated stir plate. Bring the temperature up to 60° C. and continue to stir.

Load the heated ethanol solution into a rotary evaporator. Raise the temperature to 80° C. The ethanol should start to condense and collect at this temperature. If needed, introduce vacuum at 700 micron and continue until no more ethanol is visible on the condenser. Allow to cool to room temperature. After cooling, pull the vacuum down to 500 micron and allow it to sit overnight to ensure all ethanol is removed.

After resting, heat the film back to 50° C. Introduce 1000 g of reverse osmosis water and allow it to spin on the rotary evaporator to activate the invasomes. Once complete, bottle and store.

### Example—Ethanol Free Topical Formulation

500 g of ethyl lactate is added to a beaker on a heated stir plate. The ethyl lactate is continuously stirred while being warmed to 60° C. Once at 60° C., 10 g of 95% phosphatidylcholine is dissolved into the ethanol. Next, 300 g of a lipophilic ingredient is heated in an oven to 60° C. and added to the hot ethyl lactate/PC solution. After it is homogenized, 250 g of PEG-40 Hydrogenated Castor Oil is added to the solution. Through stirring and heat, the PEG will disperse completely into the ethanol. After complete dissolution add 10 g of d-limonene terpenes to the solution. Continue to stir for an additional 20 minutes.

### Example—Natural Topical Formulation

630 g of absolute ethanol is added to a beaker on a heated stir plate. The ethanol is continuously stirred while being warmed to 60° C. Once at 60° C., 10 g of 95% phosphatidylcholine is dissolved into the ethanol. Next, 100 g of a lipophilic ingredient is heated in an oven to 60° C. and added to the hot ethanol/PC solution. After it is homogenized, 250 g of Decyl Glucoside is added to the solution. Through stirring and heat, the Decyl Glucoside will disperse completely into the ethanol. After complete dissolution, a solution of 10 g of d-limonene terpenes are added to the main ethanol beaker. Allow to stir for an additional 20 minutes at temperature.

### Example—Glyceride Formulation

620 g of absolute ethanol is added to a beaker on a heated stir plate. The ethanol is continuously stirred while being warmed to 60° C. Once at 60° C., 10 g of Capful MCM and 10 g of Captex 355 are dissolved into the ethanol. Next, 150 g of a lipophilic ingredient is heated in an oven to 60° C. and added to the hot ethanol/PC solution. After it is homogenized, 150 g of Poloxamer 407 is added to the solution. Through stirring and heat, the Poloxamer will disperse completely into the ethanol. After complete dissolution, a solution of 25 g glycerin, 10 g of d-limonene terpenes, 15 g cherry tart extract, and 10 g of a proprietary natural flavor blend are added to the main ethanol beaker. Allow to stir for an additional 20 minutes at temperature.

### Example—Sub 100 nm Invasome Formulation

Take the solution that was formulated in Example 1 and pour it into a beaker on a heated stirplate. Bring the temperature up to 60° C. and continue to stir. Add 1000 g of reverse osmosis water to the ethanol solution to activate the invasomes.

Take the activated solution and load into the high pressure homogenizer. For one hour (4 cycles) apply 15,000 PSI to the solution. Once complete, change the pressure to 25,000 PSI and allow it to run for an additional 2 hours (4 cycles). Once complete, bottle and store.

### Example—Lyophilization (Ingestible/Topical Freeze Dried Powder)

Take the solution that was formulated in Example 1 and pour it into a beaker on a heated stir plate. Bring the temperature up to 60° C. and continue to stir. Once at temperature add 40 g of Bovine Serum Albumin (BSA) to the solution.

This solution is then prepared for the freeze spray dryer. To do so, the solution was frozen at −25° C. overnight. After freezing, the solution was loaded into the freeze dryer and run through at −20° C. for 12 hours at 350 microns of vacuum.

The finished powder has a grainy consistency and can be re-hydrated into nanoparticles at any time by adding at least equal parts powder to reverse osmosis water.

### Example—Lyophilization (Inhalable Freeze Dried Powder)

630 g of absolute ethanol is added to a beaker on a heated stir plate. The ethanol is continuously stirred while being warmed to 60° C. Once at 60° C., 10 g of 95% phosphatidylcholine is dissolved into the ethanol. Next, 100 g of a lipophilic ingredient is heated in an oven to 60° C. and added to the hot ethanol/PC solution. After it is homogenized, 250 g of PEG-40 Hydrogenated Castor Oil is added to the solution. Through stirring and heat, the PEG will disperse completely into the ethanol. After complete dissolution 10 g of d-limonene terpenes and 40 g of mannitol are added to the main ethanol beaker. Allow to stir for an additional 20 minutes at temperature.

This solution is then prepared for the freeze spray dryer. To do so, the solution was frozen at −25° C. overnight. After freezing, the solution was loaded into the freeze dryer and run through at −20° C. for 12 hours at 350 microns of vacuum.

The finished powder has a grainy consistency and can be loaded into a metered dose inhaler, dry powder inhaler, or a nebulizer for delivery.

### Example—Recipe 1 (PEG Coated)

Mix: 50% Absolute Ethanol; 5% Phosphatidylcholine (PC) (main phospholipid in lecithin); 10% Cannabinoid; 27% Kolliphor RH-40 (PEG-40); 2% Glycerine; 2% Cherry Tart Extract; 4% Natural Flavor Blend.

The product is then subject to one or more of the following processes:
1. Rotovaped to remove ethanol and make a gel like substance that can be rehydrated with water;
2. High pressure homogenized to reduce size and further improve clarity;
3. Freeze dried to remove ethanol (this would require adding either mannitol or bovine serum albumin).

### Example—Recipe 2 (Polymer Coated

Mix: 50% Absolute Ethanol; 5% Phosphatidylcholine (PC); 25% Cannabinoid; 12% Poloxamer 407; 2% Glycerine; 2% Cherry Tart Extract; 4% Natural Flavor Blend.

The product is then subject to one or more of the following processes:
1. Rotovaped to remove ethanol and make a gel like substance that can be rehydrated with water;
2. High pressure homogenized to reduce size and further improve clarity;
3. Freeze dried to remove ethanol (this would require adding either mannitol or bovine serum albumin).

### Preferred Embodiments—Δ8-THC Manufacture

The process described covers industrial scale isomerization of Cannabidiol (CBD) into an essentially pure Δ8-tetrahydrocannabinol (Δ8-THC) extract. The chemical processes herein provides for making Δ8-tetrahydrocannabinol from cannabidiol extract of industrial hemp having less than 0.3% Δ9-tetrahydrocannabinol using processes for obtaining Δ8-THC that do not permit isomerization of cannabidiol to Δ9-tetrahydrocannabinol to maintain compliance with federal laws throughout processing, and using a differential distillation using vacuum distillation for volatile or low temperature impurities and wipe film distillation to remove high temperature impurities.

Essentially pure is defined as greater than 99% presence of Δ8-tetrahydrocannabinol on a weight to weight basis as detected by HPLC. Such purity of Δ8-tetrahydrocannabinol is generally accepted as a pharmaceutical, nutraceutical, skin care and/or cosmetic compositions. Additionally, the method consists of the ability not only to produce high purity Δ8-tetrahydrocannabinol (i.e. 90% to 99.9%) but also to scale up from converting hundreds of grams of CBD to the ability to convert hundreds of kilograms of CBD while maintaining said high Δ8-tetrahydrocannabinol (i.e. 90% to 99.9%). In essence the purity of said Δ8-tetrahydrocannabinol is considered essentially pure (i.e. 90% to 99.9%) on a weight to weight percent basis of the total composition.

Stated herein are the preferred and alternative methods for converting CBD to Δ8-THC. The reaction mixture can be manipulated by time, temperature, and catalyst concentration to produce extracts at different purities depending on the goal of the reaction.

Provided herein is a method of converting CBD to an essentially pure Δ8-THC with potency greater than 99%. This process is completed by introducing Cannabidiol from industrial hemp and adding it to a specific organic solvent with a specific catalyst to form a reaction mixture, loading the mixture into a reaction vessel, heating the solution to the preferred temperature, allowing it to reflux for the preferred duration, quenching the reaction mixture when complete, removing the aqueous phase, recovering the solvent, stripping the terpenes and distilling the crude residue to form a pure Δ8-THC extract.

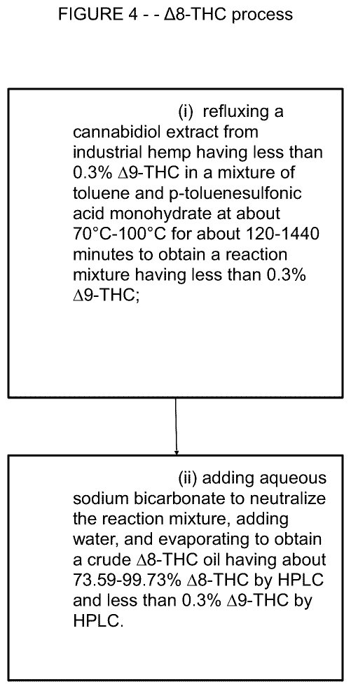

In a preferred embodiment, as shown in FIG. 4, the invention relates to a process, having the steps of:
(i) refluxing a cannabidiol extract from industrial hemp having less than 0.3% Δ9-THC in a mixture of toluene and p-toluenesulfonic acid at about 70° C.-100° C. for about 120-1440 minutes to obtain a reaction mixture having less than 0.3% Δ9-THC; and
(ii) adding aqueous sodium bicarbonate to neutralize the reaction mixture, adding water, and evaporating to obtain a crude Δ8-THC oil having about 73.59-99.73% Δ8-THC by HPLC and less than 0.3% Δ9-THC by HPLC.

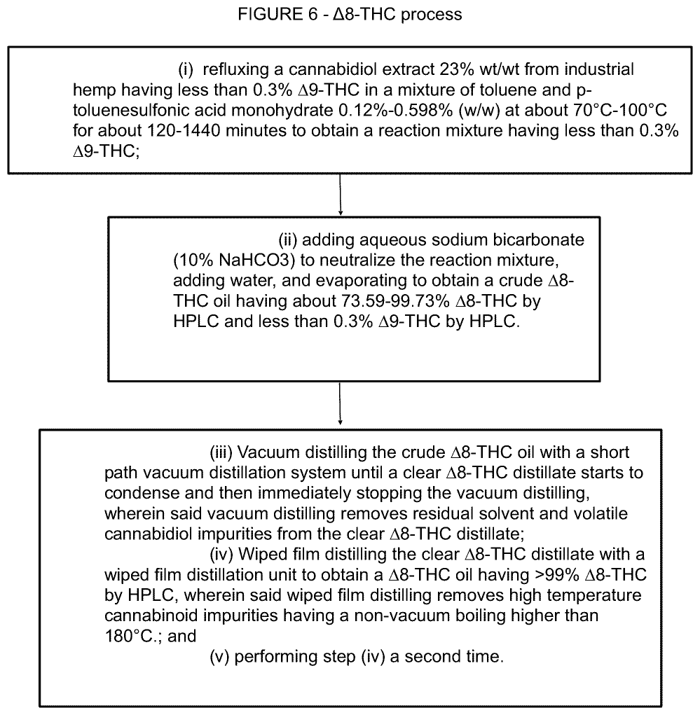

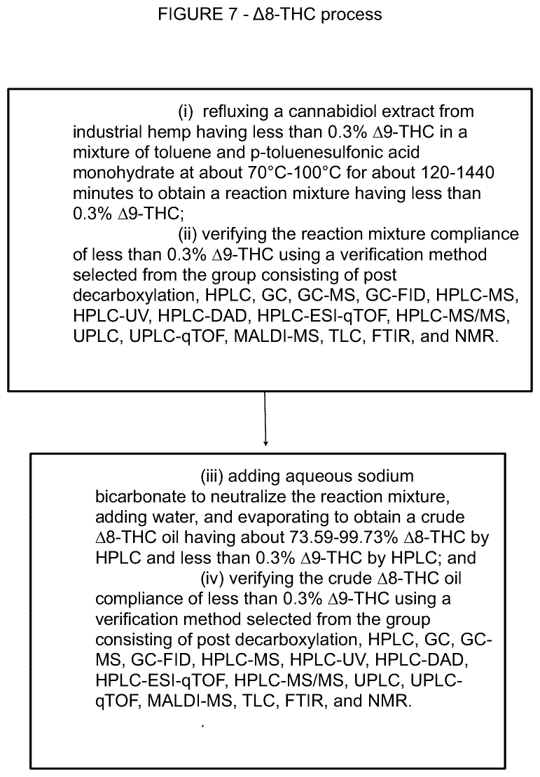

In another preferred embodiment, as shown in FIG. 6, the invention provides a process as described wherein the p-toluenesulfonic acid is about 0.12-0.598% (w/w) and the cannabidiol extract starting material is about 23% (wt/wt).

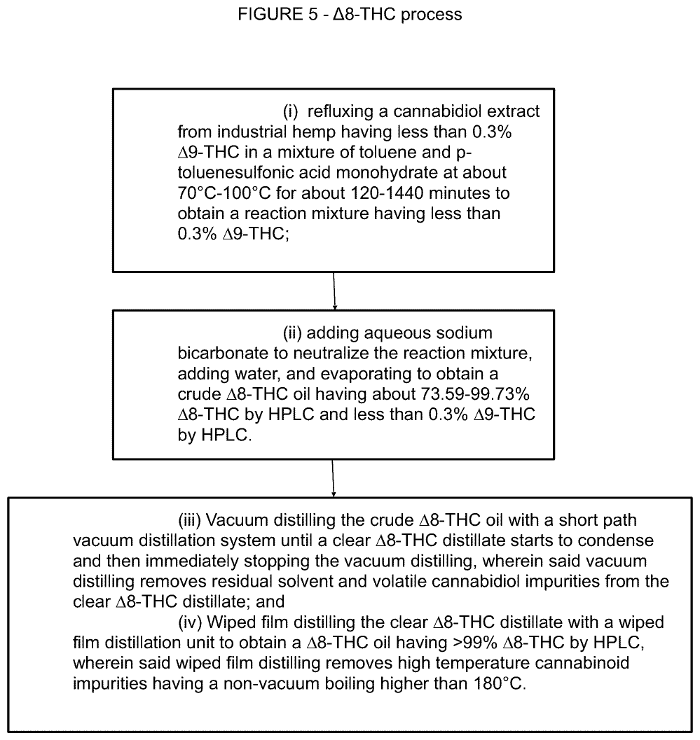

In another preferred embodiment, as shown in FIG. 5, the invention provides a process as described, comprising the additional steps of:
(iii) Vacuum distilling the crude Δ8-THC oil with a short path vacuum distillation system until a clear Δ8-THC distillate starts to condense and then immediately stopping the vacuum distilling, wherein said vacuum distilling removes residual solvent and volatile cannabidiol impurities from the clear Δ8-THC distillate; and
(iv) Wiped film distilling the clear Δ8-THC distillate with a wiped film distillation unit to obtain a Δ8-THC oil having >99% Δ8-THC by HPLC, wherein said wiped film distilling removes high temperature cannabinoid impurities having a non-vacuum boiling higher than 180° C.

In another preferred embodiment, as shown in FIG. 6, the wiped film distilling is performed twice.

Any of the preferred embodiments herein may include wherein the source of cannabidiol extract is selected from the group consisting of CBD crude, CBD distillate, and CBD isolate, and wherein the mixture is refluxed at 70° C. for 120 minutes, the aqueous sodium bicarbonate is 10% NaHCO₃, and the crude Δ8-THC oil is 91.68%-99.73% Δ8-THC by HPLC.

Any of the preferred embodiments herein may include wherein the mixture includes a second organic solvent selected from the group consisting of dichloromethane, dichloroethane, ethanol, cyclohexane, hexanes, heptanes, and a combination thereof, and wherein the mixture includes a second catalyst selected from the group consisting of Zinc Chloride, Hydrochloric acid, Sulfuric acid, Zinc Bromide, Boron Trifluoride, Boron Trifuluoride Diethyl Ethereate, and a combination thereof.

In a preferred embodiment, the invention includes a process of producing Δ8-tetrahydrocannabinol (Δ8-THC), comprising providing a source of Cannabidiol extract, adding a catalyst and organic solvent to create a reaction mixture, refluxing the reaction mixture for a specified time under acidic conditions, neutralizing the reaction, recovering a solvent product, and distilling the solvent product to obtain a crude Δ8-THC oil having >99% purity.

Any of the preferred Δ8-THC embodiments herein may include a method wherein the refluxing is selected from the group consisting of a broad reflux performed for between 0.5 to about 48 hours, a medium range reflux performed for between 60 to 180 min, and a specific reflux performed for approximately 120 min., and wherein the resulting crude Δ8-THC oil is further purified using fractional, vacuum, short path, molecular, and/or wiped film distillation.

Any of the preferred Δ8-THC embodiments herein may include wherein the dilution ratio of the Cannabinoid extract to the organic solvent is 3 to 6 on a weight basis.

Any of the preferred Δ8-THC embodiments herein may include wherein the source Cannabidiol extract is CBD crude, CBD isolate or CBD distillate, wherein the organic solvent is toluene, wherein the catalyst is 2.6% of p-toluenesulfonic acid monohydrate, and wherein the refluxing is performed for between 60 to 180 minutes at a reaction temperature selected from the group consisting of a range between 50 to 100° C., a range between 60° C. to 80° C., and approximately 70° C.

Any of the preferred Δ8-THC embodiments herein may include wherein the crude Δ8-THC having >99% purity is eluted with a second solvent or solvent mixture and separated from Δ9-THC on a Normal Phase HPLC column or a Reverse Phase HPLC column, following washing the column with the second solvent or solvent mixture, wherein the second solvent or solvent mixture is selected from toluene, ether in petroleum ether, and water-acetonitrile, wherein the eluting solvent or solvent mixture is the same as the washing solvent or solvent mixture.

Any of the preferred Δ8-THC embodiments herein may include wherein the organic solvent consists essentially of dichloromethane, dichloroethane, ethanol, cyclohexane, hexanes, heptanes, toluene, and a combination thereof.

Any of the preferred Δ8-THC embodiments herein may include wherein the catalyst is selected from the group consisting of Zinc Chloride or Hydrochloric acid or Sulfuric acid or Zinc Bromide or Boron Trifluoride or Boron Trifluoride Diethyl Ethereate, p-toluenesulfonic acid monohydrate, and a combination thereof.

Any of the preferred Δ8-THC embodiments herein may include wherein the acidic reaction mixture is neutralized using a quenching agent followed by addition of purified water, the quenching agent selected from the group consisting of sodium carbonate, sodium bicarbonate, sodium sulfate, sodium thiosulfate, a 10% NaHCO₃ solution, and a combination thereof.

In another preferred Δ8-THC embodiment, the invention provides a process, comprising: (i) dissolving 5 kg to 500 kg of CBD isolate with 25 to 250 liters of toluene to form a solution; (ii) loading the solution into a reaction vessel and heating; (iii) adding p-toluenesulfonic acid monohydrate (100 to 2 kg) to the reaction vessel and refluxing at 60-80° C. for 100-150 minutes; (iv) quenching the mixture with aqueous 10% NaHCO₃, and then adding purified water; (v) evaporating the mixture to collect a crude oil having greater than 90% Δ8-THC; (vi) loading the crude oil into a short path vacuum distillation system having Raschig rings in a condensing head and heating to remove residual solvent and terpenes and obtain a clear distillate; (vii) loading the clear distillate into a wiped film distillation unit and collecting a distilled oil having greater than 99% Δ8-tetrahydrocannabinol (Δ8-THC).

Any of the preferred Δ8-THC embodiments herein may include a pharmaceutical composition comprising the Δ8-THC made according to the processes herein and a pharmaceutically acceptable carrier, wherein the pharmaceutical composition is a topical formulation or a nutraceutical formulation.

Any of the preferred Δ8-THC embodiments herein may include a process, comprising: dissolving a quantity of CBD isolate in toluene to form a solution; loading the solution into a reaction vessel and heating; adding p-toluenesulfonic acid monohydrate to the reaction vessel and refluxing at 60-80° C. for 100-150 minutes; quenching the mixture with aqueous 10% NaHCO₃, and then adding purified water; evaporating the mixture to collect a crude oil having greater than 90% Δ8-THC; loading the crude oil into a short path vacuum distillation system having Raschig rings in a condensing head and heating to remove residual solvent and terpenes and obtain a clear distillate; loading the clear distillate into a wiped film distillation unit and collecting a distilled oil having greater than 99% Δ8-tetrahydrocannabinol (Δ8-THC).

Any of the preferred Δ8-THC embodiments herein may include an organic solvent that comprises cyclohexane, ethanol, methanol, isopropanol, acetone, toluene, hexane, pentane, heptane, methylene chloride (dichloromethane), ethylene dichloride (dichloroethane), tetrahydrofuran, benzene, chloroform, purified water, diethyl ether, and/or xylene.

In a preferred Δ8-THC embodiment, the organic solvent is toluene.

Any of the preferred Δ8-THC embodiments herein may include catalyst that may be a Lewis and/or Bronsted Lowry acid comprising acetic acid, ascorbic acid, citric acid, hydrochloric acid, hydrogen chloride, phosphoric acid, sulfuric acid, p-toluenesulfonic acid, p-toluenesulfonic acid monohydrate, zinc chloride, zinc bromide, zinc iodide, tin chloride, tin bromide, tin iodide, magnesium chloride, magnesium bromide, magnesium iodide, silver chloride, silver bromide, silver iodide, boron trifluoride, or boron trifluoride diethyl etherate,

In a preferred Δ8-THC embodiment, the catalyst is p-toluenesulfonic acid monohydrate.

In some Δ8-THC embodiments, the catalyst may be an activated powder comprising of activated carbon, bentonite clay, and/or bleaching clay.

In some Δ8-THC embodiments, the reaction mixture is filtered before being loaded into the evaporation equipment by activated carbon, bentonite clay, bleaching clay, silica, diatomaceous earth, celite, and/or mag-sil.

In some Δ8-THC embodiments, the reaction mixture is neutralized with cold water, another alkali metal hydrogen carbonate or a carbonate of an alkali metal.

In some Δ8-THC embodiments the reaction mixture is stirred at room temp, stirred while being heated or stirred while being chilled.

In some Δ8-THC embodiments Cannabidiol (CBD) isolate, distillate, crude can be used.

In some Δ8-THC embodiments the reaction can be carried out under inert atmosphere with argon, nitrogen, and/or equivalent gas.

Any of the Δ8-THC process embodiments herein may include a step of verifying compliance of less than 0.3% Δ9-THC of the crude Δ8-THC oil.

Any of the Δ8-THC process embodiments herein may include a step of verifying compliance of less than 0.3% Δ9-THC of the crude Δ8-THC oil, and comprising another step of verifying compliance of less than 0.3% Δ9-THC of the clear Δ8-THC distillate and/or Δ8-THC oil.

Any of the Δ8-THC embodiments herein may include a process for obtaining a crude Δ8-THC oil having about 73.59-99.73% Δ8-THC by HPLC and less than Δ9-THC by HPLC, then performing vacuum distillation of the crude Δ8-THC oil with a short path vacuum distillation system to obtain a clear Δ8-THC distillate, followed by a wiped film distillation of the clear Δ8-THC distillate to obtain a Δ8-THC oil having >99% Δ8-THC by HPLC, optionally followed by repeating the wiped film distillation a second time, and including the step of verifying compliance of less than 0.3% Δ9-THC of the crude Δ8-THC oil, the clear Δ8-THC distillate and/or Δ8-THC oil using a verification method selected from the group consisting of post decarboxylation, HPLC, gas chromatography (GC), GC coupled with mass spectrometry (MS), GC coupled with flame ionization detection (FID), HPLC with MS, HPLC with ultraviolet (UV) absorbance, HPLC with diode array detection (DAD), HPLC-electrospray ionization-quadrupole time of flight (ESI-qTOF), HPLC-MS/MS, ultra-performance liquid chromatography (UPLC), UPLC-qTOF, matrix assisted laser desorption ionization mass spectrometry (MALDI-MS), thin layer chromatography (TLC), Fourier transform infrared spectroscopy (FTIR), and nuclear magnetic resonance spectrometry (NMR).

### Specific Definitions

The terms Delta-8-THC or Δ8-tetrahydrocannabinol or Δ8-THC refers to

6,6,9-trimethyl-3-pentyl-6a,7,10,10a-tetrahydrobenzo[c]chromen-1-ol (IUPAC 2019-06). Delta-8-THC can be represented by 2D structure as follows:

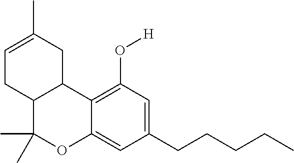 [SMILES: CCCCCc1cc(O)c2c(c1)OC(C)(C)C1CC=C(C)CC21]

The term delta-9-THC or Δ9-tetrahydrocannabinol or Δ9-THC refers to

(6aR,10aR)-6,6,9-trimethyl-3-pentyl-6a,7,8,10a-tetrahydrobenzo[c]chromen-1-ol (IUPAC 2019-06). Delta-9-THC can be represented by 2D structure as follows:

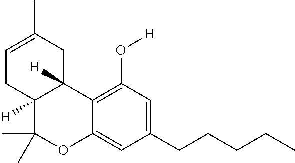

The term “pure” or “essentially pure” or “highly pure” refers to greater than 99% of Delta-8-THC in a given final product. Purity may be obtained using HPLC.

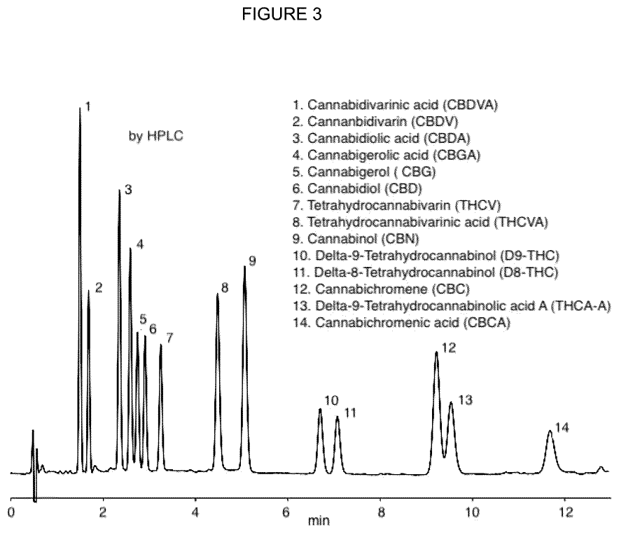

FIG. 3 illustrates, using an HPLC chromatograph, the peaks of various cannabinoids. The first to come off at around 90 seconds is cannabidivarinic acid, followed by cannabidivarin at around 1′45″. The next peaks, 3-4-5-6-7, fall between 2 minutes and 3 minutes (2′-3′) are cannabidiolic acid, cannabigerolic acid, cannabigerol, cannabidiol, and tetrahydrocannabivarin, respectively. Between 4′ and 5′, the peaks for 8 and 9 are shown for tetrahydrocannabivarinic acid, and cannabidiol (CBD). Between 6′ and 7′, the peaks for 10 and 11 are Delta-9-tetrahydrocannabinol (D9-THC), and Delta-8-tetrahydrocannabinol (D8-THC). At around the 9 minute (9′) mark the number 12 and 13 species are cannabichromene and Delta-9-tetrahydrocannabinolic acid A. And finally at about 11′30″, the cannabichromenic acid comes off.

The term “CBD” refers to cannibidiol and has a molecular weight of 314.47 g/mol.

The term “CBD Distillate” refers to the process of applying high heat (boiling point) to raw extracted oil in a distillation chamber to separate the oil components and obtain highly pure CBD. CBD distillate does not contain or contains only a very small percentage of terpenes.

The term “CBD Isolate” refers to 99% pure CBD created by cooling and crystallizing CBD extract to form a white powder

The term “hemp” does not include marijuana, and “natural hemp”, “industrial hemp”, or “hemp” as used herein refers to a variety of Cannabis sativa that contains less than 0.3% Delta-9-tetrahydrocannabinol (THC).

The term “cannabinoid” or “cannabinoids” as used herein encompasses at least the following substances: Δ-8 tetrahydrocannabinol, Δ-9-tetrahydrocannabinol (THC), cannabinol (CBN), cannabidiol (CBD), cannabigerol (CBG), Δ-9(11)-tetrahydrocannabinol (exo-THC), cannabichromene (CBC), tetrahydrocannabinol-C3 (THC-C3), tetrahydrocannabinol (THC-C4).

Examples of cannadinoids include:

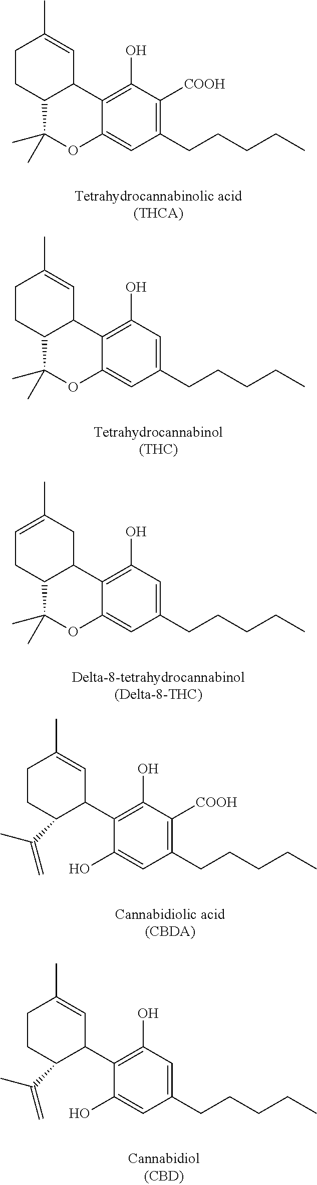

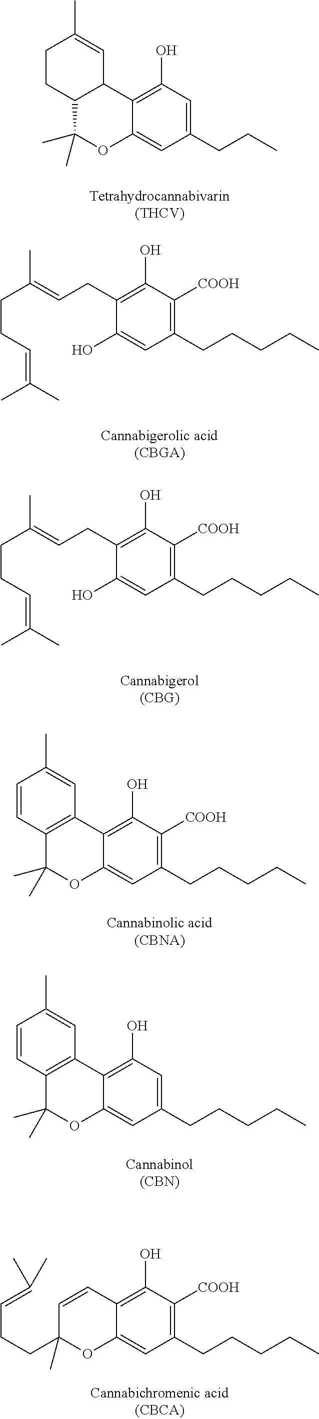

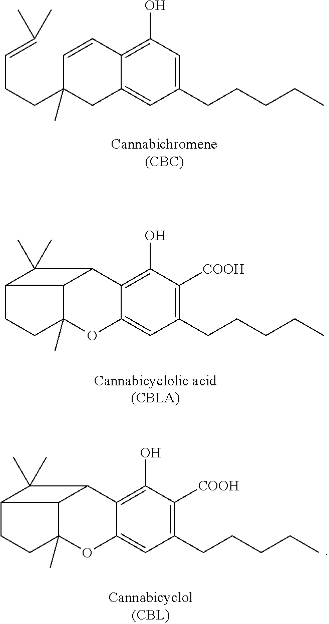 [SMILES: CCCCCc1cc(O)c2c(c1)CC(C)(CCC=C(C)C)C=C2 ; CCCCCc1cc2c(c(O)c1C(=O)O)C1C3C(CCC3(C)O2)C1(C)C ; CCCCCc1cc2c(c(O)c1C(=O)O)C1C3C(CCC3(C)O2)C1(C)C]

Δ8-THC has a published boiling point at about 177° C. Δ9-THC has a published boiling point at about 157° C. Solvents, non-compliant cannabinoids, and volatile cannabinoids are defined as having a boiling point less than about 160° C. In distillation, as used herein, these low temperature compounds are known as “heads”. High boiling point cannabinoids (vacuum) are defined herein as cannabinoids having a boiling point above about 180° C., and do not include, by definition Δ8-THC. In distillation, as used herein, these high temperature compounds are known as “tails”, with the “main” being Δ8-THC, its crude oils, its distillates, and its purified oils.

Boiling points differ among cannabinoids. This permits separation by distillation techniques.

TABLE
Relevant cannabinoid structures and boiling points

| STRUCTURE | NAME | B.P. |
|---|---|---|
| 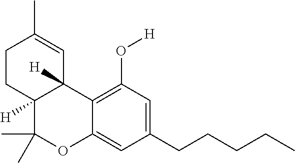 | Δ-9-THC Δ-9- tetrahydrocannabinol | 390.4° C. ± 42.0° C. at 760 mmHg; 157° C. under vacuum |
| 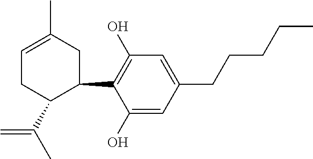 [SMILES: C=C(C)[C@@H]1CC=C(C)C[C@H]1c1c(O)cc(CCCCC)cc1O] | CBD cannabidiol | 463.9º C. ± 45.0° C. at 760 mmHg; 160-180° C. under vacuum |
| 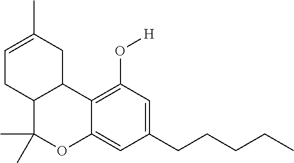 [SMILES: CCCCCc1cc(O)c2c(c1)OC(C)(C)C1CC=C(C)CC21] | Δ-8-THC Δ-8- tetrahydrocannabinol | 383.5° C. ± 42.0° C. at 760 mmHg; 175-178° C. under vacuum |

TABLE
Cannabinoid b.p. - lowest
to highest, under vacuum

| NAME | B.P. ° C. |
|---|---|
| THCA | 105 |
| CBG | 105 |
| B-CARYOPHYLLENE | 119 |
| p-CYMENE | 134 |
| a-PINENE | 156 |
| D9-THC | 157 |
| CBD | 160-180 |
| B-MYRCENE | 166-168 |
| D8-THC | 175-178 |
| 1,8-CINEOLE | 176 |
| d-LIMONENE | 177 |
| CBC | 185 |
| CBN | 185 |
| LINALOOL | 198 |
| TERPINEOL-4-OL | 209 |
| a-TERPINEOL | 218 |
| THCV | 220 |
| PULEGONE | 224 |
| APIGENIN | 270 |
| QUERCETIN | 302 |
| CBDA | 316-531 |
| B-SITOSTEROL | 414 |

The term “extraction” refers to a process for obtaining raw Cannabinoid extract from dried Hemp plant material. Non-limiting illustrative processes include CO2 extraction, liquid chromatography, solvent extraction, and olive oil extraction. Extracts contain other plant components—major and minor cannabinoids, terpenes, and flavonoids—that isolates do not.

The term “CO2 extraction” refers to a process for obtaining CBD from industrial hemp that comprises by way of illustration in a non-limiting example the following steps: —extraction with supercritical CO2 (e.g. 60° C., 250 bar); —decarboxylation (e.g. 80° C., 2 hours); and —separation in a high pressure column (using CO2 as solvent). The method is shown to yield an extract containing CBD in approximately 90% purity.

The term “Winterization” refers to combining extracted CBD oil with ethanol and freezing overnight, which is then filtered to remove fats and other impurities, and the filtrate is heated to evaporate the ethanol.

The term “Kief” refers to a high potency THC composition consisting of accumulated trichomes, or resin glands, sifted from Cannabis flowers through a mesh screen or sieve. Trichomes are the crystal-like hairs that cover the Cannabis flower bud. Trichomes secrete a sticky resin containing the terpenes and cannabinoids that give Cannabis its unique qualities. As concentrated resin glands, kief occurs as a fine powder and is a potent form of Cannabis. More simply, Kief is a Cannabis concentrate that contains from about 50%-80% THC and includes both cannabinoids and terpenes.

The term “optically active” or “chiral” CBD refers to the CBD extract comprising an optically active chiral CBD having an R,R or trans(−) rotation. CBD is known to have two chiral centers. Natural, plant-based CBD has this R,R or trans(−) rotation, specifically −125 deg. to about −129 deg. in alcohol. Contrast this with synthetic (crystalline) CBD that is not optically pure. This is due to the fact that synthetic CBD is formed from limonene, with the freebase treated with ethanol. The consequence of using limonene, which is sourced from California or Florida, is that the resulting synthetic CBD is not optically pure, and when synthetic CBD is used to form Δ9-tetrahydrocannabinol, the reaction cannot provide optically pure Δ9-tetrahydrocannabinol isomer, without additional processing to form a crystalline ester of the Δ9-tetrahydrocannabinol to obtain a single diastereomer, and then hydrolyzing back from the ester to obtain an optically pure Δ9-tetrahydrocannabinol. Thus using synthetic CBD sourced from California or Florida limonene can result in a “California isomer” or “Florida isomer” of an optically impure Δ9-tetrahydrocannabinol isomer.

The term “organic solvent” refers to ethanol, methanol, isopropanol, acetone, toluene, hexane, pentane, heptane, methylene chloride (dichloromethane), ethylene dichloride (dichloroethane), tetrahydrofuran, benzene, chloroform, purified water, diethyl ether, and/or xylene. In a preferred embodiment, the organic solvent is toluene.

The term “catalyst” for a Δ8-THC process refers to a Lewis and/or Bronsted Lowry acid comprising hydrochloric acid, hydrogen chloride, phosphoric acid, sulfuric acid, p-toluenesulfonic acid, p-toluenesulfonic acid monohydrate, zinc chloride, zinc bromide, zinc iodide, tin chloride, tin bromide, tin iodide, magnesium chloride, magnesium bromide, magnesium iodide, silver chloride, silver bromide, silver iodide, boron trifluoride, or boron trifluoride diethyl etherate, In a preferred embodiment, the catalyst is p-toluenesulfonic acid monohydrate.

The term “catalyst” for a Δ9-THC process refers to an organoaluminum catalyst is selected from the group consisting of a trialkyl- or triarylaluminum, dialkyl- or diarylaluminum halide, alkylarylaluminum halide, dialkyl- or alkylaryl- or diarylaluminum alkoxide or aryloxide, dialkyl- or alkylaryl- or diarylaluminum thioalkoxide or thioarylate, dialkyl- or alkylaryl- or diarylaluminum carboxylate, alkyl- or arylaluminum dihalide, alkyl- or arylaluminum dialkoxide or diaryloxide or alkylaryloxide, alkyl- or arylaluminum dithioalkoxide or dithioarylate, alkyl- or arylaluminum dicarboxylate, aluminum trialkoxide or triaryloxide or mixed alkylaryloxide, aluminum triacylcarboxylate, and mixtures thereof. In a preferred embodiment, the organoaluminum catalyst is a C1-C30 alkylaluminum-based catalyst. In a more preferred embodiment, the organoaluminum-based Lewis acid catalyst is ethyl aluminum dichloride, diethylaluminum chloride, diethylaluminum sesquichloride, isobutylaluminum dichloride, diisobutylaluminum chloride, or mixtures thereof. In another preferred embodiment, the trialkylaluminum is trimethylaluminum, triethylaluminum, triisopropylaluminum, triisobutylaluminum, trioctylaluminum, or tridecylaluminum. In another preferred embodiment, the catalyst is 1-2 molar triisobutylaluminum in hexane or 1 molar triisobutylaluminum in toluene. In another preferred embodiment, the catalyst is in an amount of from about 0.5 mol % to about 100 mol % with respect to the homogenized mixture, or in an amount of from about 5 mol % to about 15 mol % with respect to the homogenized mixture.

The term “Short Path Distillation” refers to slowly heating CBD oil until extraneous substances having a different boiling point than CBD, such as heads (terpenes and high volatiles), and tails (high boiling point cannabinoids), are vaporized into a distillation tube, condensed by cooling coils, and separated, leaving purified CBD oil. Short Path distillation is generally not known for scalability into large batches. Short path distillation produces a high-quality distillate, but is limited in scale.

Short path distillation utilizes an apparatus with a multi-position receiver and condensing head. This process is very limited in scale and production, but can produce high-quality distillate with an experienced operator. Crude oil is heated in a boiling ask with a magnetic stirrer. The condensing head is jacketed and requires a recirculating chiller to cool the condensing head to condense the cannabinoid vapor back into a liquid form, with the different fractions condensing into different receiving flasks.

A short path will typically have 3 fractions—heads (terpenes and high volatiles), main body (THC/CBD), and tails (high boiling point cannabinoids).

The term “Thin Film Distillation” or “Wipe Film Distillation” refers to adding CBD crude oil, under vacuum, to the top of a heated vertical cylinder on a rotating plate. As the oil enters the cylinder (a jacketed, chilled condensing head), it encounters the rotating, specially designed wipers or rollers that create and renew a thin film on the heated surface. A long, condenser in the middle of the wipers in the evaporator body, cooled with recirculating fluid, condenses the vapor. Receiving vessels collect the distillate and the high temperature residue at the bottom. A recirculating heater maintains the temperature of the feed container and outer jacketed wiped film evaporator body. Refrigerated circulators cool the condenser and cold trap.

Optimizing the feed rate, vacuum, and temperatures is essential to yield the desired component composition in the distillate. This method reduces the exposure time of the oil. With a wiped film extraction, two passes through the system are required to achieve a distillate. As in distillation, wiped film strips the crude of low boiling point compounds first, for example, terpenes and leftover volatiles. Then, during the second pass, the residue is run again to achieve the final CBD distillate.

The term “hexanes” refers to mixed isomers of hexane used as a solvent. The boiling point of hexanes is 68-70 degrees Celsius.

The term “verification” or “compliance” refers to quantitative methods for ensuring a level of less than 0.3% Δ9-THC of the starting material, the reaction intermediates and reaction mixtures, the crude Δ8-THC oil, the clear Δ8-THC distillate, and the highly pure Δ8-THC oil.

Quantitative compliance verification methods contemplated as within the scope of the invention include, without limitation, a method selected from the group consisting of post decarboxylation, HPLC, gas chromatography (GC), GC coupled with mass spectrometry (MS), GC coupled with flame ionization detection (FID), HPLC with MS, HPLC with ultraviolet (UV) absorbance, HPLC with diode array detection (DAD), HPLC-electrospray ionization-quadrupole time of flight (ESI-qTOF), HPLC-MS/MS, ultra-performance liquid chromatography (UPLC), UPLC-qTOF, matrix assisted laser desorption ionization mass spectrometry (MALDI-MS), thin layer chromatography (TLC), Fourier transform infrared spectroscopy (FTIR), and nuclear magnetic resonance spectrometry (NMR).

The term “HPLC” refers to high performance liquid chromatography.

The term “normal phase chromatography” is a type of HPLC technique. It separates analytes based on the degree of interaction towards the absorbent, which is polar silica. Therefore, the stationary phase of this type of chromatography is hydrophilic. It can also make hydrophilic interactions with the hydrophilic molecules in the sample mixture. Generally, these interactions include hydrogen bonding, dipole-dipole interactions, etc. Therefore, more non-polar analytes stay longer in the stationary phase, increasing the retention time. Furthermore, the mobile phase in the normal phase chromatography is non-polar and non-aqueous. Therefore, non-polar or hydrophobic analytes in the mixture wash out effectively with the mobile phase at the beginning of the process. Meanwhile, the retention time of analytes reduces with the increasing polarity of the mobile phase. In the present invention, it is contemplated in one embodiment that normal phase chromatography may be used for analysis of the reaction products and purified products from the intermediate steps of the process up to, but not including, the final purification step of the process.

The term “reverse-phase chromatography” is a type of recent HPLC. It has an increased reproducibility of the retention time when compared to normal phase chromatography. Basically, this increase of the reproducibility is achieved by making the stationary phase non-polar. To do that, the surface of the silica stationary phase is modified as R-(Me)2SiCl, where R is a straight-chain alkyl group such as C18H37 or C8H17. However, due to the non-polar nature of the stationary phase, less polar analytes in the sample mixture tend to have a higher retention time in contrast to the normal phase chromatography. Moreover, one can increase the retention time by adding more water to the mobile phase, which, in turn, increases the hydrophobic interactions between the non-polar analytes and the stationary phase. Also, the mobile phase of the reverse phase chromatography is polar, washing out polar analytes in the sample mixture. This facilitates the separation of the non-polar analytes in the sample mixture. Furthermore, the surface tension of the mobile phase, as well as its pH, have effects on the retention time. In the present invention, it is contemplated in one embodiment that reverse phase chromatography may be used for analysis of the final purified product from the final step of the purification process.

In contrast, normal phase chromatography refers to a separation method which allows the distribution of components of a mixture between two phases, one of which is a polar stationary phase while the mobile phase is non-polar, whereas reverse phase chromatography refers to the separation method, whose mobile phase is more polar than the stationary phase. Normal phase chromatography uses a polar stationary phase, which is mainly pure silica, while reverse phase chromatography uses a non-polar stationary phase, which is a modified silica substrate with long hydrophobic long chains. Normal phase chromatography uses a non-polar, non-aqueous solvent as the mobile phase, which is mainly chloroform while reverse phase chromatography uses a polar mobile phase, which is mainly water, methanol or acetonitrile. Normal phase chromatography separates polar analytes with high retention time in the column, while reverse phase chromatography separates less polar analytes, which have a high retention time in the column. In normal phase chromatography, the mobile phase carries non-polar analytes at the beginning of the separation while in reverse phase chromatography, the mobile phase carries polar analytes. A non-polar mobile phase increases the retention time of normal phase chromatography while a polar mobile phase increases the retention time of reverse phase chromatography. Analytes can be eluted by increasing the polarity of the mobile phase in the normal phase chromatography while the analytes can be eluted by decreasing the polarity of the mobile phase in the reverse phase chromatography. The stationary phase of normal phase chromatography contains a layer of water or protic organic solvent while the stationary phase of reverse phase chromatography does not contain water or a layer of protic solvent.

### Process Examples—Δ8-Thc

The invention will now be described by means of examples, although the invention is not limited to these examples.

TABLE 1
Summary, Examples of Conversion of CBD to Δ8-THC
Summary of Examples

| | | | Catalyst-to-solvent | | | | | |
|---|---|---|---|---|---|---|---|---|
| | Solvent | Catalyst | % | Time | Temp ° C. | Δ8 | Δ9 | CBD |
| Inventive Example 1 | Toluene | P-Tosic | 2.6 | 120 | 70 | 99.73 | N/D | N/D |
| Comparative Example 2 | Toluene | P-Tosic | 2.6 | 120 | 100 | 73.59 | N/D | N/D |
| Comparative Example 3 | Toluene | P-Tosic | 1 | 1440 | 100 | 87.01 | N/D | N/D |
| Comparative Example 4 | Toluene | P-Tosic | 1 | 2880 | 100 | 88.13 | 9.18 | N/D |
| Comparative Example 5 | Toluene | P-Tosic | 20 | 35 | 100 | 88.28 | 7.98 | N/D |
| Comparative Example 6 | Toluene | P-Tosic | 20 | 90 | 100 | 84.88 | 4.51 | N/D |
| Comparative Example 7 | Ethanol | P-Tosic | 3 | 120/30 | 30/60 | 3.20 | 2.49 | 89.43 |
| Comparative Example 8 | DCE | ZnCl2 | 250 | 1440 | 80 | 56.01 | 18.92 | N/D |
| Comparative Example 9 | DCE | ZnCl2 | 200 | 1440 | 80 | 62.13 | 30.55 | N/D |
| Comparative Example 10 | DCE | ZnCl2 | 200 | 720 | 80 | 14.36 | 27.73 | 40.41 |

### Example 1—Δ8-THC Process

CBD isolate (5000 g) was added and dissolved into toluene (25 L) to create a homogenized mixture. The solution was loaded into the reaction vessel and heat was added. P-toluenesulfonic acid monohydrate (130 g) was introduced to the reaction vessel. The mixture was refluxed at 70° C. for 120 minutes, although other time periods may be used, as discussed in alternative embodiments. The solution was then quenched with aqueous 10% NaHCO₃, then with purified water, and evaporated. The collected crude oil showed the presence of 91.68% Δ8-THC by HPLC. This extract was then loaded into a distillation unit and purified through distillation. Raschig rings were used in the distillation heads to increase purity. The completed residue was clear and stable at room temperature. The collected distilled oil showed the presence of 99.73% Δ8-THC by HPLC. An example of an essentially pure Δ8-THC by HPLC is shown in FIG. 1

In example 1, CBD isolate may have a non-limiting molar mass of 314.47 g/mol with 5000 g equivalent to about 15.90 moles. Toluene may have a molar mass of 92.14 g/mol, and 25 liters of toluene at a density of 0.87 g/mL is about 21750 g, and is equivalent to about 236.05 moles. P-toluenesulfonic acid monohydrate has a molar mass of about 172.2 g/mol, and 130 g of p-tosic is equivalent to about 0.755 moles.

The calculated mass fraction of CBD to toluene to p-tosic is:

5000 g+21750 g+130 g=26880 g, or 18.60%.+80.92%+0.48%, respectively.

The calculated weight/weight ratios of grams solute in grams solvent is:

5000 g CBD isolate in 21750 g toluene=22.99 wt %

130 g p-tosic in 21750 g toluene=0.598 wt %

The calculated mole fraction is:

15.9 mol CBD isolate/252.705 mol total=6.29% (mole fraction),

236.5 mol toluene/252.705 mol total=93.41% (mole fraction), and

0.755 mol p-tosic/252.705 mol total=0.299% (mole fraction).

### Comparative Examples—Δ8-THC Process

Following are some comparative examples, i.e. failures, that show that only a variation of the process parameters will unexpectedly result in a failed product. Decreasing or increasing the % of the catalyst will change the amount of the Δ8-THC and the Δ9-THC in the final product. This is critical because anything above Δ9-THC, by law, is a non-compliant product and must be destroyed. Similarly, changing the reflux time will change the amount of the Δ8-THC and the Δ9-THC in the final product. And changing the reflux temperature will change the amount of the Δ8-THC and the Δ9-THC in the final product. Further, performing a thermal distillation versus a vacuum short-path distillation will degrade various cannabinoids and result in higher impurities. And, letting the vacuum distillation run past the point where solvent and volatile, low boiling point cannabinoids are removed risks reducing yield of a Δ8-THC final product. Similarly, using only a single distillation process, e.g. without using the wiped film distillation, will leave high boiling point cannabinoids as impurities. As stated, these specific process parameters are critical because anything above 0.3% Δ9-THC, by law, is a non-compliant product and must be destroyed.

### Δ8-THC Process Remaining Δ9-THC Compliant

It is important to note that the inventive process starts with an industrial hemp plant that is less than 0.3% Δ9-THC. The cannabidiol (CBD) extract obtained from the compliant hemp is processed to also have less than 0.3% Δ9-THC. The next step of processing with an organic acid, e.g. p-toluenesulfonic acid, in a chemically-related and compatible solvent, e.g. toluene, followed by quenching with a neutralizing compound, e.g. sodium bicarbonate, and washing with water, also yields a crude Δ8-THC oil having less than 0.3% Δ9-THC. Thus, the entire process stays Δ9-THC-compliant at each step. Further performing a short-path vacuum distillation to remove the low temperature impurities ensures that the crude Δ8-THC oil produces a Δ8-THC distillate without allowing the cannabidiol to isomerize to the unwanted and non-compliant Δ9-THC. Lastly, performing a wiped film distillation to remove the high temperature impurities also ensures that the Δ8-THC distillate produces a highly pure Δ8-THC oil having >99% Δ8-THC by HPLC without allowing any further isomerization to the unwanted and non-compliant Δ9-THC.

### Example 8.2—Δ8-THC Comparative Failure

CBD isolate (5000 g) was added and dissolved into toluene (25 L) to create a homogenized mixture. The solution was loaded into the reaction vessel and heat was added. P-toluenesulfonic acid monohydrate (130 g) was introduced to the reaction vessel. The mixture was refluxed at 100° C. for 120 minutes, although other time periods may be used, as discussed in alternative embodiments. The solution was then quenched with aqueous 10% NaHCO₃, then with water, and evaporated. The collected crude oil showed the presence of 65.61% Δ8-THC by HPLC. This extract was then loaded into a distillation unit and purified through distillation. The completed residue was a light yellow color and stable at room temperature.

The collected distilled oil showed the presence of 73.59% Δ8-THC by HPLC.

### Example 8.3—Δ8-THC Comparative Failure

CBD isolate (5000 g) was added and dissolved into toluene (25 L) to create a homogenized mixture. The solution was loaded into the reaction vessel and heat was added. P-toluenesulfonic acid monohydrate (50 g) was introduced to the reaction vessel. The mixture was refluxed for 1440 minutes, although other time periods may be used, as discussed in alternative embodiments. The solution was then quenched with aqueous 10% NaHCO₃, then with water, and evaporated. The collected crude oil showed the presence of 81.27% Δ8-THC by HPLC. This extract was then loaded into a distillation unit and purified through distillation.

The completed residue was clear and stable at room temperature. The collected distilled oil showed the presence of 87.01% Δ8-THC by HPLC.

### Example 8.4—Δ8-THC Comparative Failure

CBD isolate (5000 g) was added and dissolved into toluene (25 L) to create a homogenized mixture. The solution was loaded into the reaction vessel and heat was added. P-toluenesulfonic acid monohydrate (50 g) was introduced to the reaction vessel. The mixture was refluxed for 2880 minutes, although other time periods may be used, as discussed in alternative embodiments. The solution was then quenched with aqueous 10% NaHCO₃, then with water, and evaporated. The collected crude oil showed the presence of 76.38% Δ8-THC but also had 5.26% Δ9-THC by HPLC. This extract was then loaded into a distillation unit and purified through distillation. The completed residue was clear and stable at room temperature. The collected distilled oil showed the presence of 88.13% Δ8-THC and 9.18% Δ9-THC by HPLC.

### Example 8.5—Δ8-THC Comparative Failure

CBD isolate (5000 g) was added and dissolved into toluene (25 L) to create a homogenized mixture. The solution was loaded into the reaction vessel and heat was added. P-toluenesulfonic acid monohydrate (1000 g) was introduced to the reaction vessel. The mixture was refluxed at 100° C. for 35 minutes, although other time periods may be used, as discussed in alternative embodiments. The solution was then quenched with aqueous 10% NaHCO₃, then with water, and evaporated. The collected crude oil showed the presence of 79.31% Δ8-THC but also had 4.32% Δ9-THC by HPLC. This extract was then loaded into a distillation unit and purified through distillation. The completed residue was a light yellow color and stable at room temperature. The collected distilled oil showed the presence of 88.28% Δ8-THC and 7.98% Δ9-THC by HPLC.

### Example 8.6—Δ8-THC Comparative Failure

CBD isolate (5000 g) was added and dissolved into toluene (25 L) to create a homogenized mixture. The solution was loaded into the reaction vessel and heat was added. P-toluenesulfonic acid monohydrate (1000 g) was introduced to the reaction vessel. The mixture was refluxed at 100° C. for 90 minutes, although other time periods may be used, as discussed in alternative embodiments. The solution was then quenched with aqueous 10% NaHCO₃, then with water, and evaporated. The collected crude oil showed the presence of 74.90% Δ8-THC but also had 2.92% Δ9-THC by HPLC. This extract was then loaded into a distillation unit and purified through distillation. The completed residue was a light yellow color and stable at room temperature. The collected distilled oil showed the presence of 84.88% Δ8-THC and 4.51% Δ9-THC by HPLC.

### Example 8.7—Δ8-THC Comparative Failure

CBD isolate (5000 g) was added and dissolved into ethanol (25 L) to create a homogenized mixture. The solution was loaded into the reaction vessel and heat was added. P-toluenesulfonic acid monohydrate (150 g) was introduced to the reaction vessel. The mixture was refluxed at 30° C. for 120 minutes, then refluxed at 60° C. for 30 minutes although other time periods may be used, as discussed in alternative embodiments. The solution was then quenched with aqueous 10% NaHCO₃, then with water, and evaporated. The collected crude oil showed the presence of 2.92% Δ8-THC but also had 2.45% Δ9-THC and 85.87% CBD by HPLC. This extract was then loaded into a distillation unit and purified through distillation. The completed residue was a light yellow color and stable at room temperature. The collected distilled oil showed the presence of 3.20% Δ8-THC, 2.49% Δ9-THC, and 89.43% CBD by HPLC.

### Example 8.8—Δ8-THC Comparative Failure

269. CBD isolate (5000 g) was added and dissolved into dichloroethane (25 L) to create a homogenized mixture. The solution was loaded into the reaction vessel and heat was added. Zinc Chloride (12500 g) was introduced to the reaction vessel. The mixture was refluxed at 80° C. for 1440 minutes, although other time periods may be used, as discussed in alternative embodiments. The solution was then quenched with aqueous 10% NaHCO₃, then with water, and evaporated. The collected crude oil showed the presence of 49.54% Δ8-THC but also had 15.25% Δ9-THC by HPLC. This extract was then loaded into a distillation unit and purified through distillation. The completed residue was a light yellow color and stable at room temperature. The collected distilled oil showed the presence of 56.01% Δ8-THC and 18.92% Δ9-THC by HPLC.

### Example 8.9—Δ8-THC Comparative Failure

CBD isolate (5000 g) was added and dissolved into dichloroethane (25 L) to create a homogenized mixture. The solution was loaded into the reaction vessel and heat was added. Zinc Chloride (10000 g) was introduced to the reaction vessel. The mixture was refluxed at 80° C. for 1440 minutes, although other time periods may be used, as discussed in alternative embodiments. The solution was then quenched with aqueous 10% NaHCO₃, then with water, and evaporated. The collected crude oil showed the presence of 54.20% Δ8-THC but also had 25.93% Δ9-THC by HPLC. This extract was then loaded into a distillation unit and purified through distillation. The completed residue was a light yellow color and stable at room temperature. The collected distilled oil showed the presence of 62.13% Δ8-THC and 30.55% Δ9-THC by HPLC.

### Example 8.10—Δ8-THC Comparative Failure

CBD isolate (5000 g) was added and dissolved into dichloroethane (25 L) to create a homogenized mixture. The solution was loaded into the reaction vessel and heat was added. Zinc Chloride (10000 g) was introduced to the reaction vessel. The mixture was refluxed at 80° C. for 720 minutes, although other time periods may be used, as discussed in alternative embodiments. The solution was then quenched with aqueous 10% NaHCO₃, then with water, and evaporated. The collected crude oil showed the presence of 12.20% Δ8-THC but also had 24.09% Δ9-THC and 37.84% CBD by HPLC. This extract was then loaded into a distillation unit and purified through distillation. The completed residue was a light yellow color and stable at room temperature. The collected distilled oil showed the presence of 14.36% Δ8-THC, 27.73% Δ9-THC, and 40.41% CBD by HPLC.

### Example 8.11—Δ8-THC Another Preferred Embodiment

CBD isolate (5000 g) was added and dissolved into toluene (25 L) to create a homogenized mixture. The solution was loaded into the reaction vessel and heat was added.

P-toluenesulfonic acid monohydrate (130 g) was introduced to the reaction vessel. The mixture was refluxed at 70° C. for 120 minutes, although other time periods may be used, as discussed in alternative embodiments. The solution was then quenched with aqueous 10% NaHCO₃, then with purified water, and evaporated. After the majority of the solvent has been evaporated, the collected crude oil showed the presence of 91.41% Δ8-THC by HPLC. The crude is then loaded into a short path vacuum distillation system. The temperature is adjusted until the residual solvent and terpenes are collected. Once the clear distillate starts to condense, the system is turned off, and the remaining crude is loaded into a wiped film distillation unit.

Parameters are set, and the material is run through the system in a much more efficient manner to establish the industrial scale processing desired by large manufacturers. Raschig rings were used in the short path distillation head to increase purity. The completed residue was clear and stable at room temperature. The collected distilled oil showed the presence of 99.68% Δ8-THC by HPLC.

### Example 8.12—Δ8-THC Another Preferred Embodiment

CBD isolate (50 kg) was added and dissolved into toluene (250 L) to create a homogenized mixture. The solution was loaded into the reaction vessel and heat was added. P-toluenesulfonic acid monohydrate (1300 g) was introduced to the reaction vessel. The mixture was refluxed at 70° C. for 120 minutes, although other time periods may be used, as discussed in alternative embodiments. The solution was then quenched with aqueous 10% NaHCO₃, then with purified water, and evaporated. After the majority of the solvent has been evaporated, the collected crude oil showed the presence of greater than 90% Δ8-THC by HPLC. The crude is then loaded into a short path vacuum distillation system. The temperature is adjusted until the residual solvent and terpenes are collected. Once the clear distillate starts to condense, the system is turned off, and the remaining crude is loaded into a wiped film distillation unit. Parameters are set, and the material is run through the system in a much more efficient manner to establish the industrial scale processing desired by large manufacturers.

Raschig rings were used in the short path distillation head to increase purity. The completed residue was clear and stable at room temperature. The collected distilled oil showed the presence of greater than 99% Δ8-THC by HPLC.

### Example 8.13—Δ8-THC Compliance Verification Embodiment

Crude Δ8-THC oil having about 73.59-99.73% Δ8-THC by HPLC and less than 0.3% Δ9-THC by HPLC is obtained using a process described herein. Vacuum distillation of the crude Δ8-THC oil with a short path vacuum distillation system to obtain a clear Δ8-THC distillate is followed by a wiped film distillation of the clear Δ8-THC distillate to obtain a Δ8-THC oil having >99% Δ8-THC by HPLC, optionally followed by repeating the wiped film distillation a second time. The step of verifying compliance of less than 0.3% Δ9-THC of the crude Δ8-THC oil, the clear Δ8-THC distillate and/or Δ8-THC oil is performed at one or more points in the process using a verification method selected from the group consisting of post decarboxylation, HPLC, gas chromatography (GC), GC coupled with mass spectrometry (MS), GC coupled with flame ionization detection (FID), HPLC with MS, HPLC with ultraviolet (UV) absorbance, HPLC with diode array detection (DAD), HPLC-electrospray ionization-quadrupole time of flight (ESI-qT0F), HPLC-MS/MS, ultra-performance liquid chromatography (UPLC), UPLC-qT0F, matrix assisted laser desorption ionization mass spectrometry (MALDI-MS), thin layer chromatography (TLC), Fourier transform infrared spectroscopy (FTIR), and nuclear magnetic resonance spectrometry (NMR).

### Preferred Embodiments—Δ9-THC

Provided herein are methods for obtaining CBD from industrial hemp plant having 0.3% or less Δ9-THC (also known as federally compliant hemp) and converting the CBD to a highly pure Δ9-THC. The reaction mixture can be manipulated by time, temperature, and catalyst concentration to produce oils at different purities depending on the goal of the reaction. The method of converting CBD yields an essentially pure Δ9-THC with potency greater than 90%. This process is completed by introducing cannabidiol and adding it to an organic solvent with a catalyst to form a reaction mixture, loading the mixture into a reaction vessel, heating the solution to the preferred temperature, allowing it to reflux for the preferred duration, quenching the reaction mixture when complete, removing the aqueous phase, recovering the solvent, stripping the terpenes and distilling the crude residue to form an essentially pure Δ9-THC oil.

Essentially pure is defined as greater than 90% presence of Δ9-THC on a weight to weight basis as detected by HPLC. Such purity of Δ9-THC is generally accepted as a pharmaceutical, nutraceutical, skin care and/or cosmetic compositions. Additionally, the method consists of the ability not only to produce high purity Δ9-THC (i.e. 90% to 99.9%) but also to scale up from converting hundreds of grams of CBD to the ability to convert hundreds of kilograms of CBD while maintaining said high purity Δ9-THC (i.e. 90% to 99.9%). In essence the purity of said Δ9-THC is considered essentially pure (i.e. 90% to 99.9%) on a weight to weight percent basis of the total composition.

More specifically, in one Δ9-THC process embodiment the invention relates to a process having the steps comprising: obtaining CBD distillate or isolate from industrial hemp plant that is less than 0.3% Δ9-THC, dissolving CBD distillate or CBD isolate in dichloromethane (DCM) to create a homogenized mixture; adding the homogenized mixture to a reactor vessel continuously purged with an inert gas and adding a 10 mol % solution of organoaluminum catalyst in hexane slowly over 30 minutes at a temperature of 18-26° C. to create a reaction mixture; stirring the reaction mixture for approximately 6-20 hours at a temperature of −20° C. to about 70° C.; quenching the reaction mixture with water or a C2-C4 alcohol, and stirring for 1 hour; filtering the reaction mixture through a filter of diatomaceous earth, perlite, or cellulose to collect a filtrate, and rinsing the filter and reaction vessel with a rinse solvent selected from dichloromethane, hexanes, or a combination of both, and combining the filtrate and the rinse solvent to obtain a combined filtrate and rinse; and performing a split path distillation of the combined filtrate and rinse, wherein the split path distillation comprises a short path distillation and a wiped film distillation to remove terpenes, high volatiles, or high boiling point cannabinoids from the combined filtrate and rinse, to obtain a Δ9-THC oil comprising over 90% Δ9-THC and trace amounts of CBD.

In another preferred Δ9-THC process embodiment, the process comprises wherein the CBD isolate is extracted from natural hemp containing 0.3% of less Δ9-THC, wherein the solvent is dichloromethane; wherein the inert gas is Argon gas or Nitrogen gas; wherein the organoaluminum catalyst is triisobutylaluminum (iBu3Al); wherein quenching uses water; wherein the filter is a diatomaceous earth filter; wherein split path distillation comprises short path distillation first to obtain a main portion separated from a heads portion and a tails portion, followed by wiped film distillation of the main portion; and, wherein the Δ9-THC oil comprises 95% or greater Δ9-THC and 3% or less unreacted CBD.

In another preferred Δ9-THC process embodiment, the process comprises wherein the CBD isolate is extracted from natural hemp containing 0.3% of less Δ9-THC, wherein the solvent is dichloromethane; wherein the inert gas is Argon gas or Nitrogen gas; wherein the organoaluminum catalyst is triisobutylaluminum (iBu3Al); wherein quenching uses water followed by sodium hydroxide followed by additional water; wherein the filter is a diatomaceous earth filter; wherein split path distillation comprises short path distillation first to obtain a main portion separated from a heads portion and a tails portion, followed by wiped film distillation of the main portion; and, wherein the Δ9-THC oil comprises 95% or greater Δ9-THC and 3% or less unreacted CBD.

In another preferred Δ9-THC process embodiment, the process comprises wherein the CBD isolate is extracted from natural hemp containing 0.3% of less Δ9-THC, wherein the solvent is dichloromethane; wherein the inert gas is Argon gas or Nitrogen gas; wherein the organoaluminum catalyst is triisobutylaluminum (iBu3Al); wherein quenching uses water followed by base such as sodium hydroxide or potassium hydroxide followed by aqueous ammonia followed by additional water to afford a granular filterable precipitate; wherein the filter is a diatomaceous earth filter; wherein split path distillation comprises short path distillation first to obtain a main portion separated from a heads portion and a tails portion, followed by wiped film distillation of the main portion; and, wherein the Δ9-THC oil comprises 95% or greater Δ9-THC and 3% or less unreacted CBD.

In a preferred Δ9-THC process embodiment, the invention relates to a process for the preparation of a high purity Δ9-tetrahydrocannabinol (Δ9-THC) product compound of the formula shown in FORMULA 1 describing the chemical structure of Δ9-THC.

The final high purity Δ9-tetrahydrocannabinol (Δ9-THC) is preferably derived from Cannabidiol isolate (CBD isolate) described in FORMULA 2.

It can also be derived from CBD Distillate or a combination thereof. As the scale of these reactions increases, the control over the process becomes more difficult, due to the exothermic reaction that results from such mixtures. The combination of a CBD isolate solution in a solvent such as dichloromethane (DCM) with the slow addition of an organoaluminum catalyst, results in much higher levels of Δ9-THC than other metal catalyst or acid previously tested such as aluminum chloride or boron trifluoride diethyl etherate. Running the reaction in a solvent such as dichloromethane (DCM) at reflux temperatures below its boiling point, further increases the conversion of CBD to Δ9-THC. Hence, the method of the present invention, by the slow addition of an organoaluminum catalyst, such as triisobutylaluminum (iBu3Al) in hydrocarbon solution, in a CBD isolate that is dissolved in DCM at a temperature below its boiling point, gives vastly improved selectivities for the production of Δ9-THC over its unwanted isomers found in the prior art. The temperature of the reaction may occur at room temperature or a temperature below the boiling point of DCM to maximize the rate of conversion of Δ9-THC.

Further, the cyclization of cannabidiol to Δ9-THC is a notoriously difficult reaction to control and carry out selectively. Previously, catalysts, such as BF3OEt2, (boron trifluoride diethyl etherate) or aluminum chloride have been used. These can induce isomerization of the desired Δ9-THC isomer to the thermodynamically more stable Δ8-THC isomer, which is very difficult to separate from the product. Moreover, cyclization of the phenol unit can occur onto the endocyclic double bond to give significant levels of iso-THC derivatives, which are also very difficult to remove. The method of the present invention, by using organoaluminum-based Lewis acid catalysts, gives vastly superior selectivities in this cyclization. For example, with boron trifluoride diethyl etherate, yields of Δ9-THC are approximately 50-60% at best, with ca. 20% iso-THC and the inherent problem of isomerization of the Δ9-THC to the Δ8-THC isomer by the strong Lewis acid. Extended reaction time favors the double bond isomerization to Δ8-THC. In contrast, when the method of the present invention is used as described herein, e.g., when triisobutylaluminum in hydrocarbon solution is used, yields of Δ9-THC are >90% with <2% iso-THC with practically no isomerization of the desired product to Δ8-THC.

Any of the Δ9-THC processes herein may include wherein quenching uses water followed by base such as sodium hydroxide or potassium hydroxide followed by aqueous ammonia followed by additional water to afford a granular filterable precipitate.

Any of the Δ9-THC processes herein may include a final basic adjustment step to remove alumina in the final product.

The Δ9-THC process produces an essentially pure Δ9-tetrahydrocannabinol (Δ9-THC) oil. The original CBD used for the reaction is obtained from an industrial hemp-based extract containing less than 0.3% Δ9-THC.

Cannabidiol is further dissolved in an organic solvent, such as dichloromethane, and cyclized by an organometallic compound. The resulting crude residue is further purified by short path, fractional, or vacuum distillation to produce an essentially pure Δ9-tetrahydrocannabinol oil.

The Δ9-THC procedure for converting cannabidiol (CBD) to Δ9-tetrahydrocannabinol consists of a cannabidiol added to an organic solvent with a catalyst to form a reaction mixture. The solution is loaded into a reaction vessel for processing. The reaction mixture is either cooled, held at room temperature or heated to preferred temperature below the boiling point of the solvent. The reaction mixture is mixed for preferred duration depending on the thermodynamic conditions to yield the highest levels of Δ9-THC. The reaction mixture is quenched with a neutralizing solution. The mixture is separated into an aqueous phase and organic phase. The aqueous layer is drained. The organic solvent is evaporated to leave a crude Δ9-tetrahydrocannabinol residue. The crude Δ9-THC is loaded into a boiling flask. The Rashig rings are added to the distillation head. The solution is heated up to a specific temperature. Any remaining terpenes, plant material, or solvent is condensed. Crude Δ9-THC either continues on to be distilled in short path or is loaded into wiped film for distillation. The crude residue is distilled to concentrate the Δ9-tetrahydrocannabinol (Δ9-THC) at scale. At the end of the procedure, essentially pure Δ9-tetrahydrocannabinol (Δ9-THC) oil is collected. The final composition of the product is essentially pure Δ9-THC at a purity of greater than 90% and preferably even greater than 95% on a weight to weight percent basis as measured by HPLC. The remaining composition consists of the original CBD isolate at a concentration of a few percent up to 3% on a weight to weight percent basis as measured by HPLC.

The preferred Δ9-THC process embodiment uses dichloromethane (DCM) as the organic solvent. Other solvents do not work to provide the high percentage of purity desired, >90% Δ9-THC.

Although, it is contemplated within the scope of the Δ9-THC invention to use this organoaluminum catalyst to obtain lower percentages of Δ9-THC, e.g. 20-50% in a final Δ9-THC oil. Any of these embodiments herein may include where the organic solvent comprises ethanol, methanol, isopropanol, ethyl acetate, acetone, acetonitrile, dimethylfuran, dimethyl sulfoxide, toluene, butane, hexane, pentane, heptane, methylene chloride (dichloromethane), ethylene dichloride, (dichloroethane), tetrahydrofuran, benzene, chloroform, purified water, diethyl ether, xylene, and combinations or mixtures thereof.

Any of the preferred Δ9-THC process embodiments herein may include where the catalyst may be an organoaluminum based catalyst compound comprising of triisobutylaluminum (iBu3Al) in hydrocarbon solution, triisobutylaluminum, or triethylaluminum, and diethylaluminum sesquachloride in a hydrocarbon solvent.

Any of the preferred Δ9-THC process embodiments herein may include where the organoaluminum catalyst is selected from the group consisting of a trialkyl- or triarylaluminum, dialkyl- or diarylaluminum halide, alkylarylaluminum halide, dialkyl- or alkylaryl- or diarylaluminum alkoxide or aryloxide, dialkyl- or alkylaryl- or diarylaluminum thioalkoxide or thioarylate, dialkyl- or alkylaryl- or diarylaluminum carboxylate, alkyl- or arylaluminum dihalide, alkyl- or arylaluminum dialkoxide or diaryloxide or alkylaryloxide, alkyl- or arylaluminum dithioalkoxide or dithioarylate, alkyl- or arylaluminum dicarboxylate, aluminum trialkoxide or triaryloxide or mixed alkylaryloxide, aluminum triacylcarboxylate, and mixtures thereof.

Any of the preferred Δ9-THC process embodiments herein may include where the organoaluminum catalyst is a C1-C30 alkylaluminum-based catalyst, or more specifically the organoaluminum-based Lewis acid catalyst is ethyl aluminum dichloride, diethylaluminum chloride, diethylaluminum sesquichloride, isobutylaluminum dichloride, diisobutylaluminum chloride, or mixtures thereof.

Any of the preferred Δ9-THC process embodiments herein may include where the trialkylaluminum is trimethylaluminum, triethylaluminum, triisopropylaluminum, triisobutylaluminum, trioctylaluminum, or tridecylaluminum.

Any of the preferred Δ9-THC process embodiments herein may include where the trialkylaluminum is triisobutylaluminum.

Any of the preferred Δ9-THC process embodiments herein may include where the organoaluminum catalyst is in an amount of from about 0.5 mol % to about 100 mol % with respect to the amount of CBD charged, the amount put in the reactor.

Any of the preferred Δ9-THC process embodiments herein may include where said organoaluminum catalyst in an amount of from about 5 mol % to about 15 mol % with respect to the amount of CBD charged.

Any of the preferred Δ9-THC process embodiments herein may include where the catalyst may be hydrolyzed with isopropyl alcohol, or another alcohol. The reaction can further be quenched with water.

Any of the preferred Δ9-THC process embodiments may include where quenching uses water followed by aqueous base such as sodium hydroxide or potassium hydroxide, optionally followed by aqueous ammonia, and then followed by additional water to afford a granular filterable precipitate.

Any of the preferred Δ9-THC process embodiments herein may include where the reaction mixture containing an organic and non-organic mixture is then separated and the organic fraction is further treated to remove the solvent from the desired Δ9-THC fraction. In some embodiments a separation funnel can be used to separate the organic phase. The organic fraction is filtered through celite before being loaded into the evaporation.

Any of the preferred Δ9-THC process embodiments herein may include where the reaction IS carried out under an inert atmosphere with an inert gas. In some embodiments the inert gas is nitrogen or argon, and/or equivalent gas, or a mixture of argon and nitrogen.

Any of the preferred Δ9-THC process embodiments herein may include where the process includes an additional aprotic solvent selected from toluene, hexane, heptane, xylene, chloroform, 1,2-dichloroethane, dichloromethane, or a mixture thereof, and preferably the solvent is dichloromethane.

Any of the preferred Δ9-THC process embodiments herein may include where said stirring is carried out at a temperature of from about −20° C. to about 70° C., or more particularly said stirring is carried out at a temperature of from about −10° C. to about 70° C., or said stirring is carried out at a temperature of from about 0° C. to about 40° C., or said stirring is carried out at a temperature of from about ° C. to about 35° C.

Any of the preferred Δ9-THC process embodiments herein may include where the process may comprise an additional purification method selected from the group consisting of chromatography, and countercurrent extraction.

Any of the preferred Δ9-THC process embodiments herein may include where the starting CBD distillate or CBD isolate is selected from the group consisting of a crude CBD extract, a CBD Isolate, a CBD distillate, and combinations thereof.

Any of the preferred Δ9-THC process embodiments herein may include wherein Kief is added to the CBD distillate or isolate. In a preferred embodiment, the Kief is 1-2% by weight. In other preferred embodiments, the Kief may include by weight.

Any of the preferred Δ9-THC embodiments herein may include where the invention is the Δ9-THC oil made according to the processes described and claimed herein.

Any of the preferred Δ9-THC embodiments herein may include where the invention comprises the Δ9-THC oil made by the processes herein and a pharmaceutically acceptable carrier.

Any of the preferred Δ9-THC embodiments herein may include where the invention comprises the Δ9-THC oil formulated as a pharmaceutical composition as a tincture, a gummi, or fast melt tab comprising: (i)>90% pure Δ9-THC oil at a dosage of 1-500 mg/dose, and (ii) a pharmaceutically acceptable carrier comprising a dietary wax, an optional secondary dietary oil, an optional secondary solvent and/or surfactant at 0.1-10% w/v, and an optional antioxidant, and an optional sweetener or flavorant.

Any of the preferred Δ9-THC embodiments herein may include where the invention comprises the Δ9-THC oil formulated as a pharmaceutical composition as a tincture, a gummi, or fast melt tab comprising: (i)>90% pure Δ9-THC oil at a dosage of 1-500 mg/dose, and (ii) a pharmaceutically acceptable carrier comprising: dietary wax selected from the group consisting of bees wax, plant waxes, very long chain fatty acid waxes, and mixtures thereof, a dietary oil selected from the group consisting of medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglycerides, MCT or LCT, sesame oil, vitamin E, soybean oil, vegetable oil, corn oil, olive oil, peanut oil, coconut oil, palmseed oil, and mixtures thereof, and an optional secondary solvents selected from the group consisting of a very long chain fatty alcohol (C24-C34), ethanol, glycerol, propylene glycol, and polyethylene glycols.

Any of the preferred Δ9-THC embodiments herein may include where the invention comprises a topical composition comprising the Δ9-THC oil in a topical formulation for skin care and cosmetic use.

Any of the preferred Δ9-THC embodiments herein may include where the invention comprises a topical composition comprising the Δ9-THC oil formulated as a topical formulation for skin care and cosmetic use, at a dosage of 1-500 mg/dose, said topical composition comprising: (i)>90% pure Δ9-THC oil, and (ii) a carrier formulation comprising: a self-emulsifying wax, a polyol, a fatty alcohol, a moisturizer, a hydrocarbon moisturizer/occlusive, an emulsifier, an antioxidant, and optionally a fragrance, a stabilizer, a skin conditioner, Aloe Barbadensis Leaf Juice, a surfactant, an anti-inflammatory, and a preservative.

Any of the preferred Δ9-THC embodiments herein may include where the invention comprises a topical composition comprising the Δ9-THC oil formulated as a topical formulation for skin care and cosmetic use, at a dosage of 1-500 mg/dose, said topical composition comprising: (i)>90% pure Δ9-THC oil, and (ii) a carrier formulation comprising: a self-emulsifying wax comprising glyceryl stearate, and/or PEG-100 stearate, a polyol comprising glycerin, a fatty alcohol comprising cetyl alcohol, a moisturizer comprising allantoin, a hydrocarbon moisturizer/occlusive comprising petrolatum, an emulsifier comprising steareth-21, an antioxidant comprising tocopheryl acetate, and optionally a fragrance, a stabilizer comprising xanthan gum, a skin conditioner comprising dipotassium glycyrrhizate, Aloe Barbadensis Leaf Juice, a surfactant comprising triethanolamine, an anti-inflammatory comprising bisabolol), and a preservative comprising disodium EDTA.

Any of the preferred Δ9-THC embodiments herein may include where the invention comprises a topical composition comprising the Δ9-THC oil formulated as a cream, an ointment, foam, gel, lotion, ointment, paste, spray, or solution, comprising: (i)>90% pure Δ9-THC, and a topical carrier selected from the group consisting of cream, ointment, foam, gel, lotion, ointment, paste, spray, and solution, wherein the cream, ointment, gel, lotion, ointment, paste is a water-in-oil or oil-in-water emulsion containing less than 20% water, greater than 50% hydrocarbons, waxes and/or polyols, and includes a surfactant to create a semi-solid, spreadable composition, wherein the foam is a cream or ointment packaged in a pressurized container and delivered with a gas, wherein the spray is a liquid packaged in a pressurized container and delivered with a gas, wherein the solution is a liquid packaged in a container and delivered with an alcohol.

Any of the preferred Δ9-THC embodiments herein may include where the invention comprises a nutraceutical composition comprising the Δ9-THC oil in a nutraceutical formulation.

Any of the preferred Δ9-THC embodiments herein may include where the invention comprises a method of administering the Δ9-THC oil to a patient in need thereof, comprising formulating the Δ9-THC oil as an oral or topical composition, wherein the patient has nausea, anxiety, stress, chronic pain, acute pain, or requires an appetite stimulant.

Any of the preferred Δ9-THC embodiments herein may include using a signature tracking marker that is added to a product or packaging containing the high purity Δ9-THC oil made herein.

Any of the preferred Δ9-THC embodiments herein may include a process for authenticating the Δ9-THC oil made according to the high purity process herein, by adding a signature marker to a product or packaging containing the Δ9-THC oil made according to the high purity process herein, testing the product or packaging for the presence of the signature marker, comparing a test result against a positive control and a negative control, and identifying the product or packaging as authentic if the test result matches the positive control more than the negative control.

### Specific Definitions

The terms Δ8-tetrahydrocannabinol or Δ8-THC or delta-8-tetrahydrocannbinol refers to

6,6,9-trimethyl-3-pentyl-6a,7,10,10a-tetrahydrobenzo[c]chromen-1-ol (IUPAC 2019-06). Δ8-THC can be represented by 2D structure as follows:

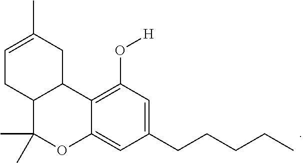 [SMILES: CCCCCc1cc(O)c2c(c1)OC(C)(C)C1CC=C(C)CC21]

The term Δ9-THC or Δ9-tetrahydrocannabinol or delta-9-tetrahydrocannbinol refers to

(6aR,10aR)-6,6,9-trimethyl-3-pentyl-6a,7,8,10a-tetrahydrobenzo[c]chromen-1-ol (IUPAC 2019-06). Δ9-THC can be represented by 2D structure as follows:

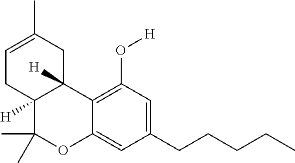

The term “pure” or “essentially pure” or “highly pure” refers to greater than 90% of Δ9-THC in the final product, and/or from 90-99% of Δ9-THC in a given final product. Purity may be obtained using HPLC.

The final high purity Δ9-tetrahydrocannabinol (Δ9-THC) is preferably derived from Cannabidiol distillate or CBD isolate, or a combination thereof. In a preferred embodiment the starting material is Cannabidiol Isolate.

As the scale of these Δ9-THC reactions increases, the control over the process becomes more difficult, considering the exothermic nature of the reaction. By adding the organoaluminum catalyst slowly, the reactions of the present invention can proceed with practically no overreaction to cyclized products. The combination of a CBD isolate solution in a solvent such as dichloromethane (DCM) with the slow addition of an organoaluminum catalyst, results in much higher levels of Δ9-THC. Running the reaction in dichloromethane (DCM) at temperatures below its boiling point further increases the conversion of CBD to Δ9-THC. Hence, the method of the present invention, by the slow addition of an organoaluminum catalyst in a CBD isolate that is dissolved in DCM at a temperature below its boiling point, gives vastly improved selectivities for the production of Δ9-THC over its unwanted isomers found in the prior art.

Cyclization of cannabidiol to Δ9-THC, without converting to the thermodynamically more stable 08-isomer, uses organoaluminum-based Lewis acid catalysts to obtain yields of Δ9-THC are >90% with <2% iso-THC with practically no isomerization of the desired product to Δ8-THC.

The organoaluminum-based Lewis acid catalyst used in the Δ9-THC method of the present invention can be a trialkyl- or triarylaluminum, dialkyl- or diarylaluminum halide, alkylarylaluminum halide, dialkyl- or alkylaryl- or diarylaluminum alkoxide or aryloxide, dialkyl- or alkylaryl or diarylaluminum thioalkoxide or thioarylate, dialkyl- or alkylaryl or diarylaluminum carboxylate, alkyl- or arylaluminum dihalide, alkyl- or arylaluminum dialkoxide or diaryloxide or alkylaryloxide, alkyl- or aryl aluminum dithioalkoxide or dithioarylate, alkyl- or arylaluminum dicarboxylate, aluminum trialkoxide or triaryloxide or mixed alkylaryloxide, aluminum triacylcarboxylate or mixtures thereof. Suitable examples of organoaluminum-based Lewis acid catalysts include, but are not limited to, trimethylaluminum, triethylaluminum, triisopropylaluminum, triisobutylaluminum, trioctylaluminum, tridecylaluminum, diethylaluminum chloride, diisobutylaluminum chloride, diethylaluminum sesquichloride, ethyl aluminum dichloride, methylaluminum dichloride, isobutylaluminum dichloride, diethylaluminum ethoxide, diethylaluminum isopropoxide, diisobutylaluminum methoxide, diisobutylaluminum phenoxide, diphenylaluminum isoproproxide, tetraisobutylalumoxane, methylalumoxane, methylaluminum bis-(2,6-di-t-butyl-4-methylphenoxide), diisobutylaluminum acetate, diisobutylaluminum benzoate, diisobutylaluminum trifluoroacetate, diisobutylaluminum isopropoxide, diisobutylaluminum 2,6-di-t-butyl-4-methylphenoxide, isobutylaluminum bis-(2,6-di-t-butyl-4-methylphenoxide), isobutylaluminum diacetate, aluminum trimethoxide, aluminum triisopropoxide, aluminum tri-tert-butoxide, and aluminum trifluoroacetate. Several such catalysts are commercially available or can be prepared from commercially available aluminum reagents, using methods known in the literature, such as described by Ooi and Maruoka, Science of Synthesis, Vol. 7, pp. 131-195, Stuttgart, Germany: Thieme (2000), which is hereby incorporated by reference in its entirety.

In one embodiment of the present Δ9-THC invention, the organoaluminum-based Lewis acid catalyst is a C1-C30 alkylaluminum-based or C6-C30 arylaluminum-based substance or mixture. In another embodiment of the present invention, the organoaluminum-based Lewis acid catalyst contains one or more oxygenated substituents bonded to the aluminum which modify the physical properties or performance of the catalyst. In another embodiment of the present invention, the organoaluminum-based Lewis acid catalyst may be made in situ before use by reaction of a precursor aluminum reagent with a modifying substituent. Specifically, the organoaluminum-based Lewis acid catalysts can be catalysts which provide high selectivity for Δ9-THC at lower levels of catalyst usage and at convenient rates for larger scale preparation. More specifically, the organoaluminum-based Lewis acid catalysts can be catalysts that produce Δ9-THC with very low levels of isomers (e.g., cis-Δ9-THC, Δ8-THC, and iso-THC), as these are difficult to remove from the product and render it difficult to achieve current standards of pharmaceutical purity.

In another embodiment of the present Δ9-THC invention, the step of treating is carried out with the organoaluminum-based Lewis acid catalyst in an amount from about 0.5 mol % to about 100 mol % with respect to the first intermediate compound. In yet another embodiment of the present invention, the step of treating is carried out with the organoaluminum-based Lewis acid catalyst in an amount from about 5 mol % to about 15 mol % with respect to the amount of CBD charged.

The step of treating can be carried out in an organic solvent. In one embodiment of the present invention, the solvent is aprotic. Examples of organic solvent include, but are not limited to ethanol, methanol, isopropanol, ethyl acetate, acetone, acetonitrile, dimethylfuran, dimethyl sulfoxide, toluene, butane, hexane, pentane, heptane, methylene chloride (dichloromethane), ethylene dichloride, (dichloroethane), tetrahydrofuran, benzene, chloroform, purified water, diethyl ether, and/or xylene and/or a mixture thereof.

The step of treating can be carried out at a temperature of from about −20° C. to the boiling point of the solvent used for the reaction. In another embodiment of the present invention, the step of treating can be carried out at a temperature of from about 0° C. to about 40° C. In yet in another embodiment of the present invention, the step of treating can be carried out at a room temperature or slightly higher than room temperature to speed up the reaction rate but cannot exceed the boiling point of the solvent. In one non-limiting embodiment, the temperature is below the boiling point of hexanes at 68-70° C. In one non-limiting embodiment, the temperature is below the boiling point of dichloromethane at 39-40° C. In one non-limiting embodiment, the temperature is below the boiling point of toluene at 110-111° C.

The starting product compound is a totally natural substance from natural hemp which contains less than 0.3% Δ9-THC as a starting material. Furthermore, the CBD is extracted from the hemp and converted to CBD distillate or CBD isolate or a combination thereof.

In another Δ9-THC embodiment, the process of the present invention further involves carrying out a method selected from chromatography, countercurrent extraction, and distillation on the product under conditions effective to produce a purified product.

### Examples—Δ9-THC

The following examples are provided to illustrate embodiments of the present invention but are by no means intended to limit its scope.

### Example—Extraction of CBD from Industrial Hemp Containing 0.3% or Less Δ9-THC

Dried Hemp plant material is processed, or extracted, to obtain a raw Cannabinoid extract. Non-limiting illustrative processes include CO2 extraction, liquid chromatography, solvent extraction, and olive oil extraction. The extract contains other plant components—major and minor cannabinoids, terpenes, and flavonoids—that isolates do not.

In a non-limiting preferred embodiment, industrial hemp containing 0.3% or less Δ9-THC is processed using “CO2 extraction” to obtain CBD. Specific steps include: —extraction with supercritical CO2 (e.g. 60° C., 250 bar); —decarboxylation (e.g. 80° C., 2 hours); and —separation in a high pressure column (using CO2 as solvent). The method yields an extract containing CBD in approximately 90% purity.

### Example—Extraction of CBD from Hemp Containing 0.3% or Less Δ9-THC, and Winterization

Industrial hemp containing 0.3% or less Δ9-THC is processed using “CO2 extraction” to obtain CBD. Specific steps include: —extraction with supercritical CO2 (e.g. 60° C., 250 bar); —decarboxylation (e.g. 80° C., 2 hours); and —separation in a high pressure column (using CO2 as solvent). The method yields an extract containing CBD in approximately 90% purity. The CBD extracted oil is combined with ethanol and is then frozen overnight. After freezing, the combined CBD-EtOH is then filtered to remove fats and other impurities, and the filtrate is heated to evaporate the ethanol.

The following examples show Δ9-THC from CBD Distillate.

### Example 9.1. Preparation of Δ9-THC from CBD Distillate Using Basic Ionic Resin Workup

CBD Distillate (15 kg @ 85% CBD content) was dissolved in dichloromethane (75 liters) in a nitrogen inerted, 150 L cylindrical glass reactor with an overhead stirrer, pressure equalizing addition funnel and a water-cooled reflux condenser to create a homogenized mixture. The reactor was inerted with nitrogen and triisobutylaluminum (6 L of 1 M solution in hexane, 10 mol % catalyst) was then slowly added over 30 minutes at a batch temperature of 26° C. The reaction mixture was stirred for 6 hours at a temperature of 26 to 30° C., at which point the reaction was determined to be complete by TLC and HPLC analysis (<2.0% CBD by HPLC). The solution was quenched with 25 liter of water and stirred for 1 hour, then circulated through a water washed Amberlyst A21 resin column for two hours. The reaction mixture was filtered through celite coated sparkler filter. The reactor and celite cake were rinsed with dichloromethane (5 L) and rinse was added to the clarified batch. The layers were separated, and the aqueous layer was washed with fresh dichloromethane (2.5 L) and this rinse was added to the organic layer. The organic layer was concentrated under reduced pressure. The remaining organic residue was wiped film distilled (165° C. at 15 mTorr vacuum) to afford 8.7 kg (58% yield when corrected for the assay of the starting material) of Δ9-THC as a clear to light yellow viscous oil. The HPLC analysis of this product was comparable to the assay of earlier batches.

### Example 9.2. Preparation of Δ9-THC from CBD Distillate, with Aqueous Sodium Hydroxide Workup

CBD Distillate (5.0 kg @ 85% CBD content) was dissolved in dichloromethane (15 liters) in a nitrogen inerted, 50 L cylindrical glass reactor with an overhead stirrer, pressure equalizing addition funnel and a water-cooled reflux condenser to create a homogenized mixture. The reactor was inerted with nitrogen and triisobutylaluminum (2.0 L of 1 M solution in hexane, 10 mol % catalyst) was then slowly added over 3.5 hours at a batch temperature between 18 and 30° C. The reaction mixture was stirred for approximately 6 hours at a temperature of 20 to ° C., at which point the reaction was determined to be complete by TLC and HPLC analysis (<2.0% CBD by HPLC). The batch was diluted with THF (4 L) and stirred for 15 minutes. The solution was quenched with water (75 mL) followed by 15% NaOH solution (75 g) and then water (235 mL). The quenched batch stirred for 1 hour, then magnesium sulfate (500 g) was added, and the stirring was continued for an additional hour. The alumina containing suspension was clarified through a celite coated sparkler filter. The filter cake was washed with fresh THF (1 L) and the wash was added to the batch. The filtrate was concentrated under reduced pressure using a rotary evaporator until the light solvents stopped distilling. The remaining organic residue was wiped film distilled in two temperature stages (first pass: 160° C. at 15 mTorr vacuum then second pass: 170° C. at 15 mTorr vacuum) to afford 3.83 kg (90% yield when corrected for the assay of the starting material) of Δ9-THC as a clear to light yellow viscous oil. The HPLC analysis of this product was comparable to the assay of earlier batches.

### Example 9.3. Preparation of Δ9-THC from CBD Distillate, with Aqueous Ammonia Workup

CBD Distillate (1.0 kg @ 85% CBD content) was dissolved in dichloromethane (3 liters) in a nitrogen inerted, 22 L glass reactor with an overhead stirrer, pressure equalizing addition funnel and a water-cooled reflux condenser to create a homogenized mixture. The reactor was inerted with nitrogen and triisobutylaluminum (400 mL of 1 M solution in hexane, 10 mol % catalyst) was then slowly added over 2 hours at a batch temperature between 18 and 30° C. The reaction mixture was stirred for approximately 6 hours at a temperature of 20 to 25° C., at which point the reaction was determined to be complete by TLC and HPLC analysis (<2.0% CBD by HPLC). The reaction was quenched with water (15 mL) and when the active catalyst was quenched, the reaction mixture was rotary evaporated under reduced pressure to ca. 1 L volume. The batch was diluted with methyl tert-butyl ether (MtBE) (2 L) and the mixture was stirred. To the stirred solution was added 28% aqueous ammonia solution (25 mL) followed by additional water (50 mL). To the resulting slurry was added sodium sulfate (100 g) and the stirring was continued for an additional hour. The batch was clarified through a celite coated sparkler filter, and the filter cake was washed with MtBE, and the wash was added to the filtered batch. The filtrate was concentrated under reduced pressure using a rotary evaporator until the light solvents stopped distilling. The remaining organic residue was wiped film distilled in two temperature stages (first pass: 160° C. at 15 mTorr vacuum then second pass: 170° C. at 15 mTorr vacuum) to afford 766 g (90% yield when corrected for the assay of the starting material) of Δ9-THC as a clear to light yellow viscous oil. The HPLC analysis of this product was comparable to the assay of earlier batches.

### Example 9.4. Preparation of Δ9-THC from CBD Distillate, with Aqueous KF Workup

CBD Distillate (500 g @ 85% CBD content) was dissolved in dichloromethane (2 liters) in a nitrogen inerted, 22 L glass reactor with an overhead stirrer, pressure equalizing addition funnel and a water-cooled reflux condenser to create a homogenized mixture. The reactor was inerted with nitrogen and triisobutylaluminum (200 mL of 1 M solution in hexane, 10 mol % catalyst) was then slowly added over 2 hours at a batch temperature between 18 and 30° C. The reaction mixture was stirred for approximately 6 hours at a temperature of 20 to 25° C., at which point the reaction was determined to be complete by TLC and HPLC analysis (<2.0% CBD by HPLC). The reaction was quenched with a mixture of potassium fluoride (28 g) in water (15 mL) and when the active catalyst was quenched, the reaction mixture was rotary evaporated under reduced pressure to ca. 0.5 L volume. The batch was diluted with methyl tert-butyl ether (MtBE) (2 L) and the mixture was stirred. To the stirred solution was added silica gel (30 g) and the stirring was continued for an additional hour. The batch was clarified through a celite coated sparkler filter, and the filter cake was washed with MtBE, and the wash was added to the filtered batch. The filtrate was concentrated under reduced pressure using a rotary evaporator until the light solvents stopped distilling. The remaining organic residue was short path distilled and the main fraction was collected at 165° C. to 175° C. at 25 mTorr vacuum to afford 228 g (80% yield when corrected for the assay of the starting material) of Δ9-THC as a clear to light yellow viscous oil. The HPLC analysis of this product was comparable to the assay of earlier batches.

### Example 9.5. Preparation of Δ9-THC from CBD Distillate, with Aqueous Workup

CBD Distillate (15 kg @ 85% CBD content) was dissolved in dichloromethane (75 liters) in a nitrogen inerted, 150 L cylindrical glass reactor with an overhead stirrer, pressure equalizing addition funnel and a water-cooled reflux condenser to create a homogenized mixture. The reactor was inerted with nitrogen and triisobutylaluminum (6 L of 1 M solution in hexane, 10 mol % catalyst) was then slowly added over 2 hours at a batch temperature of 26° C. The reaction mixture was stirred for 6 hours at a temperature of 26 to 30° C., at which point the reaction was determined to be complete by TLC and HPLC analysis (<2.0% CBD by HPLC). The reaction was quenched by pumping the batch into a 200 L reactor equipped with a bottom valve and mechanical stirrer containing water (40 L) The addition time was about 30 minutes. The biphasic mixture was stirred for an additional 2 hours. The stirring was stopped, and the layers were separated. The bottom organic layer was saved for further processing and the remaining aqueous layer was extracted fresh dichloromethane (20 L) and this wash was added to the quenched organic solution. This solution was clarified through celite coated sparkler filter. The reactor and celite cake were rinsed with dichloromethane (5 L) and rinse was added to the clarified batch. The organic layer was concentrated under reduced pressure. The remaining organic residue was wiped film distilled (165° C. at 15 mTorr vacuum) to afford 8.7 kg (58% yield when corrected for the assay of the starting material) of Δ9-THC as a clear to light yellow viscous oil. The HPLC analysis of this product was comparable to the assay of earlier batches.

### Example 9.6. Preparation of Δ9-THC from CBD Distillate Fortified with Kief, with the Aqueous Workup

CBD Distillate (15 kg @ 85% CBD content) was dissolved in dichloromethane (75 liters) in a nitrogen inerted, 150 L cylindrical glass reactor with an overhead stirrer, pressure equalizing addition funnel and a water-cooled reflux condenser to create a homogenized mixture. To this solution was added Kief (30 g, 0.2 weight %) and the mixture was stirred until the Kief dissolved. The reactor was re-inerted with nitrogen and triisobutylaluminum (4 L of 1 M solution in hexane, 6.6 mol % catalyst) was then slowly added over 2 hours at a batch temperature of 26° C. The reaction mixture was stirred for 8 hours at a temperature of 26 to 30° C., at which point the reaction was determined to be complete by TLC and HPLC analysis (<2.0% CBD by HPLC). The reaction was quenched by pumping the batch into a 200 L reactor equipped with a bottom valve and mechanical stirrer containing water (40 L). The addition time was about 30 minutes. The biphasic mixture was stirred for an additional 2 hours and celite (3.0 kg) was added. This mixture was clarified through celite coated sparkler filter. The quench reactor and celite cake were rinsed with dichloromethane (5 L) and rinse was added to the clarified biphasic mixture. The stirring was stopped, and the layers were separated. The bottom organic layer was saved for further processing and the remaining aqueous layer was extracted fresh dichloromethane (20 L) and this wash was added to the quenched organic solution. The organic solution was concentrated under reduced pressure. The remaining organic residue was wiped film distilled (165° C. at 15 mTorr vacuum) to afford 13.1 kg (87.3% yield when corrected for the assay of the starting material) of Δ9-THC as a clear to light yellow viscous oil. The HPLC analysis of this product was comparable to the assay of earlier batches.

The following examples show Δ9-THC from CBD isolate.

### Example 9.7. Preparation of Δ9-THC from CBD Isolate, Using Basic Ionic Resin Workup

CBD Isolate (185 g at >95% purity) was dissolved in dichloromethane (1 L) in an argon inerted, 5 L, 3-necked round bottom flask with an overhead stirrer, pressure equalizing addition funnel and a water-cooled condenser. Triisobutylaluminum (60 mL of 1 M solution in hexane, 10 mol % catalyst) was then slowly added over 30 minutes at about room temperature. The reaction mixture was stirred for approximately 20 hours, until the reaction was complete by TLC and HPLC analysis (<2.0% CBD by HPLC). The reaction was quenched with water (1 L) and the biphasic mixture was stirred for about 1 hour. The biphasic mixture was passed through a water washed Amberlyst A21 (mildly basic) column The biphasic reaction mixture was then clarified through a celite loaded sparkler filter. The celite cake and reaction vessels were rinsed with dichloromethane (1 L) and combined with the clarified biphasic batch mixtures. The layers were separated, and the water layer was washed with fresh dichloromethane (250 mL) The combined organic layers were transferred to a short path distillation apparatus and concentrated under reduced pressure and the residue was distilled (170° C. at 15 mTorr vacuum) to afford 135 g; (73% yield) of Δ9-THC as a clear to light yellow viscous oil. The HPLC analysis of this product showed 93% Δ9-THC and 4% unreacted CBD.

### Table—Quantitative Results

### Example 9.8. Scale Up Preparation of Δ9-THC from CBD Isolate, Using Basic Ionic Resin Workup

CBD Isolate (15 kg at >95% purity) was dissolved in dichloromethane (75 liters) in a nitrogen inerted, 150 L, cylindrical glass reactor with an overhead stirrer, pressure equalizing addition funnel and a water-cooled reflux condenser. to create a homogenized mixture. Triisobutylaluminum (3 L of 1 M solution in hexane, 10 mol % catalyst) was then slowly added over 30 minutes maintaining the reactor below 25° C. The reaction mixture was stirred for approximately 20 hours at which point the reaction was determined to be complete by TLC and HPLC analysis (<2.0% CBD by HPLC). The solution was quenched with water (25 L) and stirred for 1 hour, then circulated through a pair of water washed Amberlyst A21 resin column for two hours. The reaction mixture was then filtered through a celite loaded sparkler filter. The celite cake and reaction vessels were rinsed with dichloromethane (5 L) and combined with the total mixtures of solvents. The layers were separated, and the aqueous layer was washed with fresh dichloromethane (2.5 L) and the remaining aqueous layer was disposed. The organic solvent containing the desired product was concentrated under reduced pressure and the residue was wiped film distilled (165° C. at 15 mTorr vacuum) to afford 10.2 kg (68% yield) of Δ9-THC as a clear to light yellow viscous oil. The HPLC analysis of this product showed 93% Δ9-THC and 4% unreacted CBD.

### Example 9.9. Preparation of Δ9-THC from CBD Isolate Fortified with Kief, with the Aqueous Workup

CBD Isolate (300 g at >98% purity) was dissolved in dichloromethane (2 L) in an argon inerted, 12 L, 3-necked round bottom flask with an overhead stirrer, pressure equalizing addition funnel and a water-cooled condenser. To this solution was added Kief (3 g, 1 weight %) and the mixture was stirred until the Kief dissolved. Triisobutylaluminum (100 mL of 1 M solution in hexane, 10 mol % catalyst) was then slowly added over 30 minutes at about room temperature. The reaction mixture was stirred for approximately 20 hours, until the reaction was complete by TLC and HPLC analysis (<2.0% CBD by HPLC). The reaction was quenched with water (1 L) and the biphasic mixture was stirred for about 1 hour. Celite (66 g) was added to the batch and the slurry was stirred for 1 hour. The reaction mixture was then clarified through a celite loaded sparkler filter. The celite cake were rinsed with dichloromethane (1 L) and combined with the clarified biphasic mixtures. The layers were separated, and the water layer was washed with fresh dichloromethane (250 mL) The combined organic layers were transferred to a short path distillation apparatus and concentrated under reduced pressure and the residue was distilled (170° C. at 15 mTorr vacuum) to afford 235 g; (78% yield) of Δ9-THC as a clear to a pale-yellow viscous oil. The HPLC analysis of this product showed 93% Δ9-THC and 4% unreacted CBD.

The following examples demonstrate Δ9-THC from combined CBD distillate and isolate.

### Example 9.10. Preparation of Δ9-THC from Combined CBD Distillate and Isolate, with Aqueous Workup

CBD Distillate (7.5 kg @ 85% CBD content) and CBD isolate (7.5 kg at >95% purity) are dissolved in dichloromethane (75 liters) in a nitrogen inerted, 150 L cylindrical glass reactor with an overhead stirrer, pressure equalizing addition funnel and a water-cooled reflux condenser to create a homogenized mixture. The reactor is inerted with nitrogen and triisobutylaluminum (6 L of 1 M solution in hexane, 10 mol % catalyst) is then slowly added over 2 hours at a batch temperature of 26° C. The reaction mixture is stirred for 6 hours at a temperature of 26 to 30° C., at which point the reaction is determined to be complete by TLC and HPLC analysis (<2.0% CBD by HPLC). The reaction is quenched by pumping the batch into a 200 L reactor equipped with a bottom valve and mechanical stirrer containing water (40 L) The addition time is about 30 minutes. The biphasic mixture is stirred for an additional 2 hours. The stirring is stopped, and the layers are separated. The bottom organic layer is saved for further processing and the remaining aqueous layer is extracted fresh dichloromethane (20 L) and this wash is added to the quenched organic solution. This solution is clarified through celite coated sparkler filter. The reactor and celite cake are rinsed with dichloromethane (5 L) and rinse is added to the clarified batch. The organic layer is concentrated under reduced pressure. The remaining organic residue is wiped film distilled (165° C. at 15 mTorr vacuum) to afford 8.7 kg (58% yield when corrected for the assay of the starting material) of Δ9-THC as a clear to light yellow viscous oil. The HPLC analysis of this product is comparable to the assay of earlier batches.

### Example 9.11. Preparation of Δ9-THC from CBD Distillate and Isolate Fortified with Kief, with the Aqueous Workup

CBD Distillate (7.5 kg @ 85% CBD content) and CBD isolate (7.5 kg at >95% purity) are dissolved in dichloromethane (75 liters) in a nitrogen inerted, 150 L cylindrical glass reactor with an overhead stirrer, pressure equalizing addition funnel and a water-cooled reflux condenser to create a homogenized mixture. To this solution is added Kief (30 g, 0.2 weight %) and the mixture is stirred until the Kief is dissolved. The reactor is re-inerted with nitrogen and triisobutylaluminum (4 L of 1 M solution in hexane, 6.6 mol % catalyst) is then slowly added over 2 hours at a batch temperature of 26° C. The reaction mixture is stirred for 8 hours at a temperature of 26 to 30° C., at which point the reaction is determined to be complete by TLC and HPLC analysis (<2.0% CBD by HPLC). The reaction is quenched by pumping the batch into a 200 L reactor equipped with a bottom valve and mechanical stirrer containing water (40 L). The addition time is about 30 minutes. The biphasic mixture is stirred for an additional 2 hours and celite (3.0 kg) is added. This mixture is clarified through celite coated sparkler filter. The quench reactor and celite cake are rinsed with dichloromethane (5 L) and rinse is added to the clarified biphasic mixture. The stirring is stopped, and the layers are separated. The bottom organic layer is saved for further processing and the remaining aqueous layer is extracted fresh dichloromethane (20 L) and this wash is added to the quenched organic solution. The organic solution is concentrated under reduced pressure. The remaining organic residue is wiped film distilled (165° C. at 15 mTorr vacuum) to afford 13.1 kg (87.3% yield when corrected for the assay of the starting material) of Δ9-THC as a clear to light yellow viscous oil. The HPLC analysis of this product is comparable to the assay of earlier batches.

### Pharmaceutical Compositions/Medicaments

Any of the compositions of the invention may be converted using customary methods into pharmaceutical compositions and medicaments. The pharmaceutical composition and medicaments contain the composition of the invention either alone or together with other active substances. Such pharmaceutical compositions and medicaments can be for oral, topical, rectal, parenteral, local, or inhalant use. They are therefore in solid or semisolid form, for example oils, drops, lotions, balm, pills, tablets, creams, gelatin capsules, capsules, suppositories, soft gelatin capsules, gels, foams, powders, and formulated for internal use. For parenteral uses, those forms for intramuscular or subcutaneous administration can be used, or forms for infusion or intravenous injection can be used, and can therefore be prepared as solutions of the compositions and medicaments or as powders of the active compositions to be mixed with one or more pharmaceutically acceptable excipients or diluents, suitable for the aforesaid uses and with an osmolarity that is compatible with the physiological fluids. For local use, those preparations in the form of creams or ointments for topical use or in the form of sprays may be considered; for inhalant uses, preparations in the form of sprays, for example nose sprays, may be considered. Preferably, the composition and medicaments is administered topically or orally.

Any of the pharmaceutical compositions and medicaments can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Nack Publishing Company, Easton, Pa., USA 1985).

On this basis, the pharmaceutical compositions and medicaments include, albeit not exclusively, the composition of the invention in association with one or more pharmaceutically acceptable vehicles or diluents, and are contained in buffered solutions with a suitable pH and iso-osmotic with the physiological fluids.

Any of the compositions and medicaments are indicated as therapeutic agents either alone or in conjunction with other therapeutic agents or other forms of treatment. For example, in the case of skin care or cosmetic use, or for nausea, anxiety, stress, chronic pain, acute pain and used as an appetite stimulant. The compositions and agents of the invention are intended for administration to humans or animals.

### Example—Δ8-THC Oral Formulation

A >99% pure Δ8-THC oil having less than 0.3% Δ9-THC is prepared, the Δ8-THC oil at a dosage of 0.5-150 mg is homogenized with a dietary oil, an optional secondary solvent and/or surfactant at 0.1-10% w/v, and an optional anti-oxidant. An optional sweetener or flavorant may be added. An oral formulation of pure D8-THC is obtained. The dietary oil may comprise medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglyceride, and derivatives and mixtures thereof. The dietary oil may also comprise, alone or in combination with MCT or LCT, sesame oil, vitamin E, soybean oil, vegetable oil, corn oil, olive oil, peanut oil, coconut oil, palmseed oil, and mixtures thereof. The optional secondary solvents are selected from ethanol, glycerol, propylene glycol, and polyethylene glycols.

### Example—Δ8-THC Oral Formulation

A >99% pure Δ8-THC oil having less than 0.3% Δ9-THC is prepared, the Δ8-THC oil at a dosage of 0.5-150 mg is formulated into a tincture, a gummi, or fast melt tab, by mixing a dietary wax, an optional secondary dietary oil, an optional secondary solvent and/or surfactant at 0.1-10% w/v, and an optional anti-oxidant. An optional sweetener or flavorant may be added. An oral formulation of pure D8-THC is obtained. The dietary wax may comprise bees wax, plant waxes, very long chain fatty acid waxes, and mixtures thereof. The dietary oil may comprise medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglyceride, and derivatives and mixtures thereof. The dietary oil may also comprise, alone or in combination with MCT or LCT, sesame oil, vitamin E, soybean oil, vegetable oil, corn oil, olive oil, peanut oil, coconut oil, palmseed oil, and mixtures thereof. The optional secondary solvents are selected from a very long chain fatty alcohol (C24-C34), ethanol, glycerol, propylene glycol, and polyethylene glycols.

### Example—Δ8-THC Oral Formulation

A >99% pure Δ8-THC oil having less than 0.3% D9-THC is prepared, the Δ8-THC oil at a dosage of 0.5-150 mg is formulated into a tincture, a gummi, or fast melt tab, by mixing with sesame oil and ethanol. An oral formulation of pure Δ8-THC is obtained.

### Example—Δ8-THC Edible

An edible product comprising a composition of the present invention. Edible products include a pure Δ8-THC oil formulated in a food composition selected from an edible, a meltable form for adding to hot beverages selected from coffee, tea, cider, cocoa, and mixed hot drinks, a powder or dissolvable form for adding to cold or room temperature beverages selected from water, tea, coffee, a soda/carbonate drink, a cider, a juice, an energy drink, beer, ale, wine, a liquor, a mixed beverage, a gummy, lozenge, a candy, a hard candy, a boiled sweets, lollipop, gummy candy, candy bar, chocolate, a brownie, a cookie, a trail bar, a cracker, a dissolving strip, a mint, a pastry, a bread, and a chewing gum.

### Example—D8-THC Oral Formulation

A >99% pure Δ8-THC oil having less than 0.3% Δ9-THC is prepared, the Δ8-THC oil at a dosage of 0.5-150 mg is formulated into a tincture, a gummi, or fast melt tab, by mixing a dietary wax, an optional secondary dietary oil, an optional secondary solvent and/or surfactant at 0.1-10% w/v, and an optional anti-oxidant. An optional sweetener or flavorant may be added. An oral formulation of pure D8-THC is obtained. The dietary wax may comprise bees wax, plant waxes, very long chain fatty acid waxes, and mixtures thereof. The dietary oil may comprise medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglyceride, and derivatives and mixtures thereof. The dietary oil may also comprise, alone or in combination with MCT or LCT, sesame oil, vitamin E, soybean oil, vegetable oil, corn oil, olive oil, peanut oil, coconut oil, palmseed oil, and mixtures thereof. The optional secondary solvents are selected from a very long chain fatty alcohol (C24-C34), ethanol, glycerol, propylene glycol, and polyethylene glycols.

### Example—Δ9-THC Oral Formulation

A pure Δ9-THC oil is prepared, the Δ9-THC oil at a dosage of 0.5-150 mg is formulated into a tincture, a gummi, or fast melt tab, by mixing with sesame oil and ethanol. An oral formulation of pure Δ9-THC is obtained.

### Example—Δ9-THC Edible

An edible product comprising a composition of the present invention. Edible products include a pure Δ9-THC oil formulated in a food composition selected from an edible, a meltable form for adding to hot beverages selected from coffee, tea, cider, cocoa, and mixed hot drinks, a powder or dissolvable form for adding to cold or room temperature beverages selected from water, tea, coffee, a soda/carbonate drink, a cider, a juice, an energy drink, beer, ale, wine, a liquor, a mixed beverage, a gummy, lozenge, a candy, a hard candy, a boiled sweets, lollipop, gummy candy, candy bar, chocolate, a brownie, a cookie, a trail bar, a cracker, a dissolving strip, a mint, a pastry, a bread, and a chewing gum.

### Example—Δ9-THC Oral Formulation

A >95% pure Δ9-THC oil is prepared, the Δ9-THC oil at a dosage of 1-500 mg/dose is homogenized with a dietary oil, an optional secondary solvent and/or surfactant at 0.1-10% w/v, and an optional antioxidant. An optional sweetener or flavorant may be added. An oral formulation of pure Δ9-THC is obtained. The dietary oil may comprise medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglyceride, and derivatives and mixtures thereof. The dietary oil may also comprise, alone or in combination with mid-chain triglycerides or long-chain triglycerides, sesame oil, vitamin E, soybean oil, vegetable oil, corn oil, olive oil, peanut oil, coconut oil, palmseed oil, and mixtures thereof. The optional secondary solvents are selected from ethanol, glycerol, propylene glycol, and polyethylene glycols.

### Example Δ9-THC—Oral Formulation

A >95% pure Δ9-THC oil is prepared, the Δ9-THC oil at a dosage of 1-500 mg/dose is formulated into a tincture, a gummi, or fast melt tab, by mixing a dietary wax, an optional secondary dietary oil, an optional secondary solvent and/or surfactant at 0.1-10% w/v, and an optional antioxidant. An optional sweetener or flavorant may be added. An oral formulation of pure Δ9-THC is obtained. The dietary wax may comprise beeswax, plant waxes, very long chain fatty acid waxes, and mixtures thereof. The dietary oil may comprise medium chain (C8-C12) and long chain (C10-C22) dietary triglycerides selected from the group consisting of caprylic triglyceride, capric triglyceride, lauric triglyceride, myristic triglyceride, palmitic triglyceride, stearic triglyceride, oleic triglyceride, linoleic triglyceride, gamma linoleic triglyceride, ricinoleic triglyceride, arachidic triglyceride, behenic triglyceride, and derivatives and mixtures thereof. The dietary oil may also comprise, alone or in combination with MCT or LCT, sesame oil, vitamin E, soybean oil, vegetable oil, corn oil, olive oil, peanut oil, coconut oil, palmseed oil, and mixtures thereof. The optional secondary solvents are selected from a very long chain fatty alcohol (C24-C34), ethanol, glycerol, propylene glycol, and polyethylene glycols.

### Example Δ9-THC—Oral Formulation

A >99% pure Δ9-THC oil is prepared, the Δ9-THC oil at a dosage of 1-500 mg/dose is formulated into a tincture, a gummi, or fast melt tab, by mixing with sesame oil and ethanol. An oral formulation of pure Δ9-THC is obtained.

### Example Δ9-THC—Oral Formulation

A 90-99% pure Δ9-THC oil is prepared, the Δ9-THC oil at a dosage of 1-500 mg/dose is formulated into a food composition selected from an edible, a meltable form for adding to hot beverages selected from coffee, tea, cider, cocoa, and mixed hot drinks, a powder or dissolvable form for adding to cold or room temperature beverages selected from water, iced tea, iced coffee, a soda/carbonate drink, a cider, a juice, an energy drink, beer, ale, wine, a fermented beverage such as Kombucha and Kefir, a liquor, a mixed beverage, a gummy, a lozenge, a candy, a hard candy, a boiled sweets, lollipop, gummy candy, candy bar, chocolate, a brownie, a cookie, a trail bar, a cracker, a dissolving strip, a mint, a pastry, a bread, and a chewing gum.

### Dosages

Dosages for nanoemulsions containing Δ8-THC or Δ9-THC contemplated as within the scope of the invention include, without limitation, the following dosage examples:
1 mg to 2.5 mg Δ8-THC or Δ9-THC edibles—for mild relief of symptoms like pain, stress, and anxiety; increased focus and creativity.
2.5 mg to 15 mg Δ8-THC or Δ9-THC edibles—for stronger relief of pain and anxiety symptoms; sleep aid.
30 mg to 100 mg Δ8-THC or Δ9-THC edibles—for patients living with inflammatory disorders, cancer, and other serious conditions.

Other preferred dosages of the invention include 1 mg, 2.5 mg, 5 mg, and 10 mg capsules. For chemotherapy, as a non-limiting example, a 5 mg capsule is taken 1-3 hours before chemotherapy, and then additional 5 mg capsules every 2-4 hours as prescribed or as necessary. For anxiety, appetite increase (e.g. in people diagnosed with AIDS), opioid withdrawal, or narcotic relapse prevention, a patient may take a 1 or 2 mg tablet twice per day, as prescribed.

In another embodiment, the Δ8-THC or Δ9-THC is co-administered with CBD as a combination delivered simultaneously, or as a combination delivered sequentially. A preferred embodiment includes a ratio of Δ8-THC or Δ9-THC to CBD of about 1:2, or 1:3, or 1:4, or 1:5.

### Topical Formulations

In preferred embodiments, the present compositions can additionally comprise at least one skin conditioning agent. In this regard, the present compositions preferably contain about 1% to about 15% by weight, and more preferably from about 5% to about 10% of at least one agent. The skin conditioning agent can help provide the softening, smoothing, lubricating, and skin conditioning features of the presently preferred compositions.

Preferred non-limiting examples of skin conditioning agents useful in the present compositions include petrolatum, red petrolatum, white petrolatum, liquid petrolatum, semi-solid petrolatum, light mineral oil, heavy mineral oil, white mineral oil, mineral oil alcohols, calamine, derivatives thereof, and mixtures thereof.

### Organosiloxane

Any of the presently preferred compositions can further comprise at least one organosiloxane. Organosiloxanes useful in the present compositions can be volatile or nonvolatile, including but not limited to polyalkylsilicones, cyclic polyalkylsiloxanes, polydialkylsiloxanes, polydiarylsiloxanes, polyalkarylsiloxanes, or cyclomethicones.

Preferred polyalkylsiloxanes useful in this regard have a viscosity of from about to about 100,000 centistokes at 25.degree. C., and more preferably have a viscosity of less than 500 centistokes at 25.degree. C.

### Aqueous Solvent

Any of the present compositions additionally comprise an aqueous solvent. Preferably the aqueous solvent is present in the instant compositions from about 50% to about 95% by weight, and more preferably from about 60% to about 90% by weight.

### Emollient

Certain of the presently preferred compositions can additionally comprise at least one emollient. The present compositions may contain about 0.01% to about 5% by weight, and more preferably from about 0.1% to about 1% by weight of an emollient.

### Dermatologically Acceptable Excipients

Any of the preferred compositions discussed herein can additionally comprise at least one dermatologically acceptable excipient commonly known to those of ordinary skill in the art as useful in topical compositions. Preferred, non-limiting examples of dermatologically acceptable excipients useful in these compositions are those selected from the group consisting of moisturizers, preservatives, gelling agents, colorants or pigments, antioxidants, radical scavengers, emulsifiers, pH modifiers, chelating agents, penetration enhancers, derivatives thereof, and mixtures thereof.

### Moisturizers

Any of the presently preferred compositions may optionally further contain at least one moisturizer. Preferably, the presently preferred compositions can comprise about 0.01% to about 10% by weight of at least one moisturizer. Preferred non-limiting examples of moisturizers that can optionally be included in these compositions include glycerin, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, dipropylene glycol, sorbitol, derivatives thereof, and mixtures thereof.

### Preservatives

Any of the presently preferred compositions may optionally further contain at least one preservative. Preferred non-limiting examples of preservatives that can optionally be included in these compositions include benzyl alcohol, methyl paraben, ethyl paraben, derivatives thereof, and mixtures thereof. A particularly preferred preservative in this regard is benzyl alcohol or a derivative thereof. Additionally, the preservative is preferably present in an amount of about 0.1% to about 2.5% by weight of the overall weight of the composition.

### Gelling Agents

Any of the presently preferred compositions may optionally further contain a gelling agent. Preferred non-limiting examples of gelling agents that can optionally be included in these compositions include various cellulose agents, such as cellulosic polymers, methylcellulose, hydroxyethylcellulose, hydroxypropylcellulose, and hydroxypropylmethylcellulose. Additional, non-limiting examples of gelling agents include gum arabic, gum tragacanth, locust bean gum, guar gum, xanthan gum, cellulose gum, sodium carbomer, carbomer, polyacrylic polymers, derivatives thereof, and mixtures thereof. Other suitable gelling agents which may be useful in the present compositions include aqueous gelling agents, such as neutral, anionic, and cationic polymers, derivatives thereof, and mixtures thereof. Exemplary polymers which may be useful in the preferred compositions in this regard include carboxy vinyl polymers, such as carboxypolymethylene. Additionally preferred gelling agents include Carbopol® and Carbomer® polymers (i.e. polyacrylic polymers) such as is available from Noveon Inc., Cleveland, Ohio. The gelling agent is preferably present in the instant compositions in an amount of from about 0.01% to about 10%, more preferably from about 0.1% to about 5%, and most preferably from about 0.1% to about 2%, by weight.

### Anti-Oxidants

Any of the presently preferred compositions may optionally further contain at least one anti-oxidant. Preferably, the presently preferred compositions can comprise about 0.1% to about 5% by weight of at least one anti-oxidant. Preferred non-limiting examples of antioxidants that can optionally be included in these compositions include ascorbic acid, ascorbyl esters of fatty acids, magnesium ascorbyl phosphate, sodium ascorbyl phosphate, ascorbyl sorbate, tocopherol, tocopherol sorbate, tocopherol acetate, butylated hydroxy benzoic acid, thioglycolates, persulfate salts, 6-hydroxy-2,5,7,8-tetramethylchroman-2-carboxylic acid, lipoic acid, gallic acid, propyl gallate, uric acid, sorbic acid, lipoic acid, amines, N,N-diethylhydroxylamine, N-acetyl-L-cysteine, amino-guanidine, sulfhydryl compounds, glutathione, dihydroxy fumaric acid, lycine pidolate, arginine pilolate, nordihydroguaiaretic acid, bioflavonoids, curcumin, lysine, 1-methionine, proline, superoxide dismutase, silymarin, tea extracts, grape skin/seed extracts, melanin, rosemary extracts, derivatives thereof, and mixtures thereof.

### Emulsifiers

Any of the presently preferred compositions may optionally further contain an emulsifier. Preferably, the presently preferred compositions can comprise about to about 15% by weight, and more preferably from about 0.5% to about 10% by weight of at least one emulsifier. Preferred, non-limiting examples of specific emulsifiers useful in this regard include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-8 stearate, PEG-100 stearate, derivatives thereof, and mixtures thereof.

### pH Modifiers

Any of the presently preferred compositions may optionally further contain a pH modifier. Preferably, the presently preferred compositions can comprise about to about 1% by weight of a pH modifier. Preferred non-limiting examples of neutralizing pH modifiers that can optionally be included in these compositions include inorganic hydroxides, inorganic oxides, inorganic salts of weak acids, derivatives thereof, and mixtures thereof. Preferred, non-limiting examples of inorganic hydroxides useful in this regard include ammonium hydroxide, alkali metal hydroxide, alkaline earth metal hydroxides, derivatives thereof, and mixtures thereof. Preferred inorganic hydroxides useful in this regard include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof. Preferred, non-limiting examples of inorganic oxides useful in this regard include magnesium oxide, calcium oxide, derivatives thereof, and mixtures thereof. Preferred, non-limiting examples of inorganic salts of weak acids useful in this regard include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

### Chelating Agents

Any of the presently preferred compositions may optionally further contain a chelating agent. Preferably, the presently preferred compositions can comprise about 0.01% to about 1% by weight of a chelating agent. Preferred non-limiting examples of chelating agents that can optionally be included in these compositions include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosphonates, pharmaceutically acceptable salts thereof, derivatives thereof, and mixtures thereof.

In addition to those enumerated above, any other pharmaceutically active agent, occlusive skin conditioning agent, emollient, penetration enhancer, organosiloxane, moisturizer, preservative, gelling agent, colorant or pigment, antioxidant, radical scavenger, emulsifier, pH modifier, chelating agent, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein. Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In another particularly preferred embodiment, the presently preferred pharmaceutical compositions are formulated in a lotion, cream, ointment, gel, suspension, emulsion, foam, aerosol, or other pharmaceutically acceptable topical dosage form.

### Example—Δ8-THC Topical Transdermal Composition

A >99% pure Δ8-THC oil having less than 0.3% Δ9-THC is prepared, the Δ8-THC oil at a dosage of 0.5-150 mg is formulated into a transdermal formulation by mixing pure D8-THC with a transdermal formulation base, the transdermal formulation base comprising an emulsion formed from an aqueous phase and an oil phase, and an penetration enhancer, an optional emulsifier, and an optional emollient. A topical transdermal D8-THC composition is thereby obtained.

### Example—Δ8-THC Topical Composition

A >99% pure Δ8-THC oil having less than 0.3% Δ9-THC is prepared, the Δ8-THC oil at a dosage of 0.5-150 mg is formulated as a cream, an ointment, foam, gel, lotion, ointment, paste, spray, or solution. A topical >99% pure Δ8-THC composition having less than 0.3% Δ9-THC is thereby obtained.

The cream or ointment is a water-in-oil or oil-in-water emulsion containing less than 20% water, greater than 50% hydrocarbons, waxes and/or polyols, and using a surfactant to create a semi-solid, spreadable composition. The foam is a cream or ointment packaged in a pressurized container and delivered with a gas.

### Example—Δ8-THC Topical Composition

A >99% pure Δ8-THC oil having less than 0.3% Δ9-THC is prepared, the Δ8-THC oil at a dosage of 0.5-150 mg is formulated as a topical composition comprising: (i) >99% pure Δ8-THC oil having less than 0.3% Δ9-THC, and (ii) a carrier formulation comprising: a self-emulsifying wax (i.e. glyceryl stearate, PEG-100 stearate), a polyol (glycerin), a fatty alcohol (cetyl alcohol), a moisturizer (allantoin), a hydrocarbon moisturizer/occlusive (petrolatum), an emulsifier (i.e. steareth-21), an antioxidant (tocopheryl acetate), and optionally a fragrance, a stabilizer (xanthan gum), a skin conditioner (i.e dipotassium glycyrrhizate), Aloe Barbadensis Leaf Juice, a surfactant (triethanolamine), an anti-inflammatory (i.e. bisabolol), and a preservative (disodium EDTA).

Any of the topical formulations herein may include a hydrocarbon base (“oleaginous”), such a white petrolatum or white ointment, an absorption base (water-in-oil) such as hydrophilic petrolatum or lanolin, water-removable base (oil-in-water) such as hydrophilic ointment, or a water-soluble base, such as polyethylene glycol ointment.

The topical formulation may also include a wax such as bees wax, plant waxes, very long chain fatty acid waxes, and mixtures thereof, an oil such as medium chain (C8-C12) and long chain (C10-C22) triglycerides, and alone or in combination with MCT or LCT, sesame oil, vitamin E, soybean oil, vegetable oil, corn oil, olive oil, peanut oil, coconut oil, palmseed oil, and mixtures thereof. Any of the topical formulations herein may include solvents are selected from a very long chain fatty alcohol (C24-C34), ethanol, glycerol, propylene glycol, and polyethylene glycols. Any of the topical formulations herein may include a penetration enhancer such as ethoxydiglycol (i.e. transcutanol) or an equivalent.

### Example Δ9-THC—Topical Transdermal Composition

A >90% pure Δ9-THC oil is prepared, the Δ9-THC oil at a dosage of 1-500 mg/dose is formulated into a transdermal formulation by mixing pure Δ9-THC with a transdermal formulation base, the transdermal formulation base comprising an emulsion formed from an aqueous phase and an oil phase, and an penetration enhancer, an optional emulsifier, and an optional emollient. A topical transdermal Δ9-THC composition is thereby obtained.

### Example Δ9-THC—Topical Composition

A >95% pure Δ9-THC oil is prepared, the Δ9-THC oil at a dosage of 1-500 mg/dose is formulated as a cream, an ointment, foam, gel, lotion, ointment, paste, spray, or solution. A topical >95% pure Δ9-THC composition is thereby obtained. The cream or ointment is a water-in-oil or oil-in-water emulsion containing less than 20% water, greater than 50% hydrocarbons, waxes and/or polyols, and using a surfactant to create a semi-solid, spreadable composition. The foam is a cream or ointment packaged in a pressurized container and delivered with a gas.

### Example Δ9-THC—Topical Composition

A >99% pure Δ9-THC oil is prepared, the Δ9-THC oil at a dosage of 1-500 mg/dose is formulated as a topical composition comprising: (i)>99% pure Δ9-THC oil, and (ii) a carrier formulation comprising: a self-emulsifying wax (i.e. glyceryl stearate, PEG-100 stearate), a polyol (glycerin), a fatty alcohol (cetyl alcohol), a moisturizer (allantoin), a hydrocarbon moisturizer/occlusive (petrolatum), an emulsifier (i.e. steareth-21), an antioxidant (tocopheryl acetate), and optionally a fragrance, a stabilizer (xanthan gum), a skin conditioner (i.e dipotassium glycyrrhizate), Aloe Barbadensis Leaf Juice, a surfactant (triethanolamine), an anti-inflammatory (i.e. bisabolol), and a preservative (disodium EDTA).

Any of the topical formulations herein may include a hydrocarbon base (“oleaginous”), such a white petrolatum or white ointment, an absorption base (water-in-oil) such as hydrophilic petrolatum or lanolin, water-removable base (oil-in-water) such as hydrophilic ointment, or a water-soluble base, such as polyethylene glycol ointment.

The topical formulation may also include a wax such as beeswax, plant waxes, very long chain fatty acid waxes, and mixtures thereof, an oil such as medium chain (C8-C12) and long chain (C10-C22) triglycerides, and alone or in combination with MCT or LCT, sesame oil, vitamin E, soybean oil, vegetable oil, corn oil, olive oil, peanut oil, coconut oil, palmseed oil, and mixtures thereof. Any of the topical formulations herein may include solvents are selected from a very long chain fatty alcohol (C24-C34), ethanol, glycerol, propylene glycol, and polyethylene glycols. Any of the topical formulations herein may include a penetration enhancer such as ethoxydiglycol (i.e. transcutanol) or an equivalent.

### Uses

As used herein, the terms “administering”, “administration”, and like terms refer to any method which, in sound medical or cosmetic practice, delivers the composition to a subject in such a manner as to provide a positive effect on a dermatological disorder, condition, or appearance. The compositions are preferably administered such that they cover the entire area to be treated. “Direct administration” refers to any method which, in sound medical or cosmetic practice, delivers the composition to a subject without the use of another composition, delivery agent, or device. “Indirect administration” refers to any method which, in sound medical or cosmetic practice, delivers the composition to a subject with the use of at least one other composition, delivery agent, or device.

As used herein, the phrases an “effective amount” or a “therapeutically effective amount” of an active agent or ingredient, or pharmaceutically active agent or ingredient, which are synonymous herein, refer to an amount of the pharmaceutically active agent sufficient enough to have a positive effect on the area of application. Accordingly, these amounts are sufficient to modify the skin disorder, condition, or appearance to be treated but low enough to avoid serious side effects, within the scope of sound medical or dermatological advice. A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

### Anti-Tampering and Anti-Counterfeiting

The invention also includes adding a signature marker molecule to a product containing the high purity Δ9-THC oil made herein.

### Example—Authentication, Anti-Counterfeit Markers

Step 1. A product containing the high purity Δ9-THC oil is prepared. The product can include an over-the-counter product or a pharmaceutical composition or medicament. The product or dosage form can include manufactured for oral, topical, rectal, parenteral, local, or inhalant use. The product may be in in solid or semisolid form, and may include oils, drops, sprays, lotions, balm, pills, tablets, creams, ointments, gelatin capsules, capsules, suppositories, soft gelatin capsules, gels, foams, powders, and formulations for internal use including intramuscular forms, subcutaneous forms, infusion forms, injectable forms, reconstitutable powder forms.

Step 2. A signature marker molecule is added to the product and/or the packaging. The signature molecule marker can be a single identifier or may be specific combination of chemicals. Where the signature marker molecule is a single identifier is used, the invention contemplates the use of a DNA molecular tag such as the SigNature® molecular tag (Applied DNA Sciences, Inc.), which is a DNA based identifier added as a film to oral or topical dosage forms to form a covert authentication platform. This DNA tag functions as a “molecular bar code”, enabling identification to a source, as a product type, or other meaningful attribute. To validate the product, the product is tested for the presence of the tag using a rt-PCR kit that includes reagents and a reader, such as the reagent mix SigNify® Reagent Mix and the SigNify® IF portable reader (Applied DNA Sciences, Inc.) which uses real-time polymerase chain reaction (rt-PCR).

Where a signature marker molecule is a specific combination chemicals, the invention contemplates the use of specific trackable batches that containing specific varying amounts and types of substitutable inactive agents. Such inactive agent combinations can include one or more of a solvent, surfactant, antioxidant, triglyceride, oil, conditioning agent, organosiloxane, emollient, excipient, moisturizer, preservative, gelling agent, emulsifier, pH modifier, chelator, colorant, visible pigment and/or non-visible pigment (UV or near IR) such as a fluorescent pigment. By selecting and tracking the specific amount and type of inactive agent, the product can be authenticated.

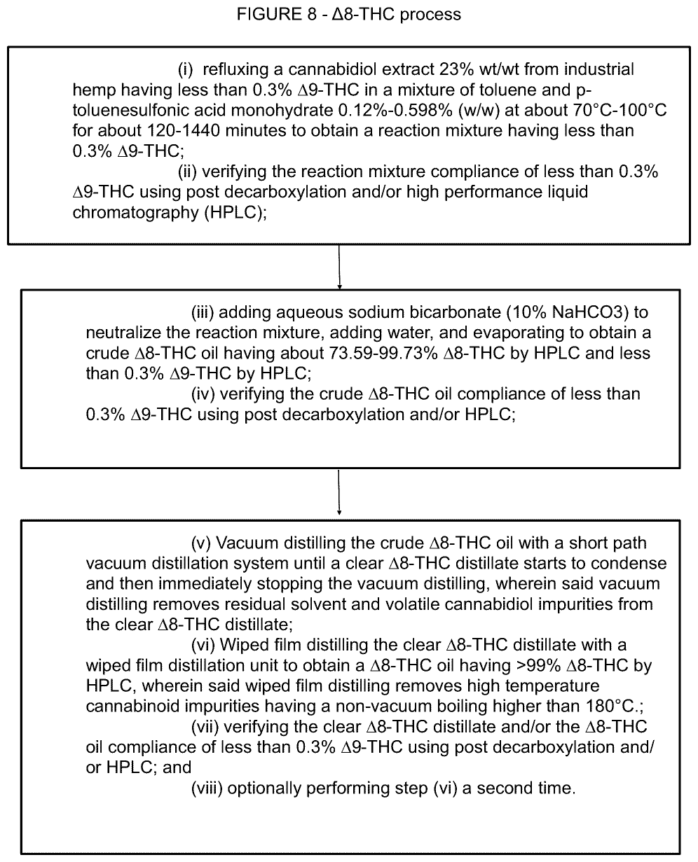

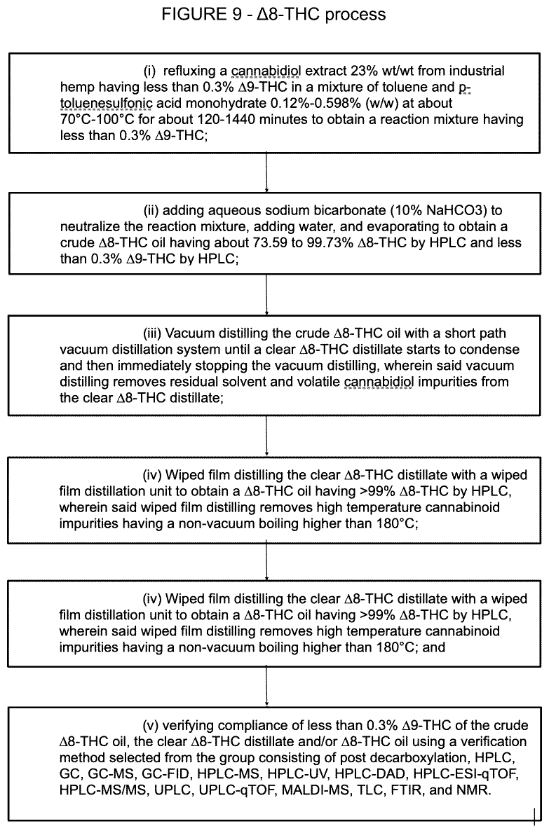

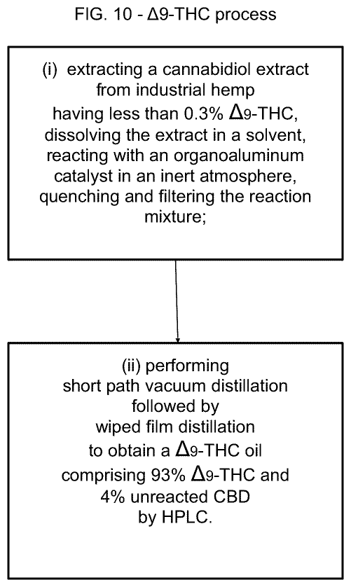

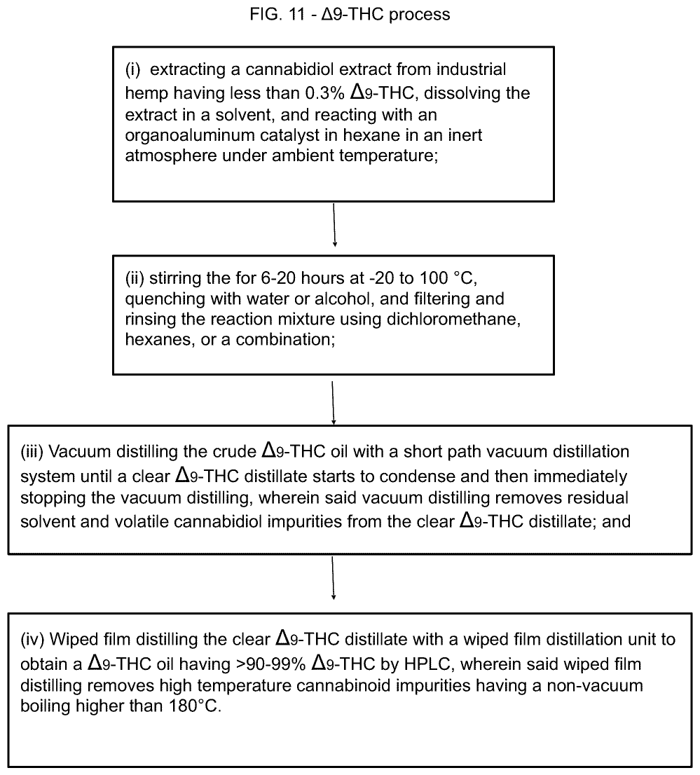

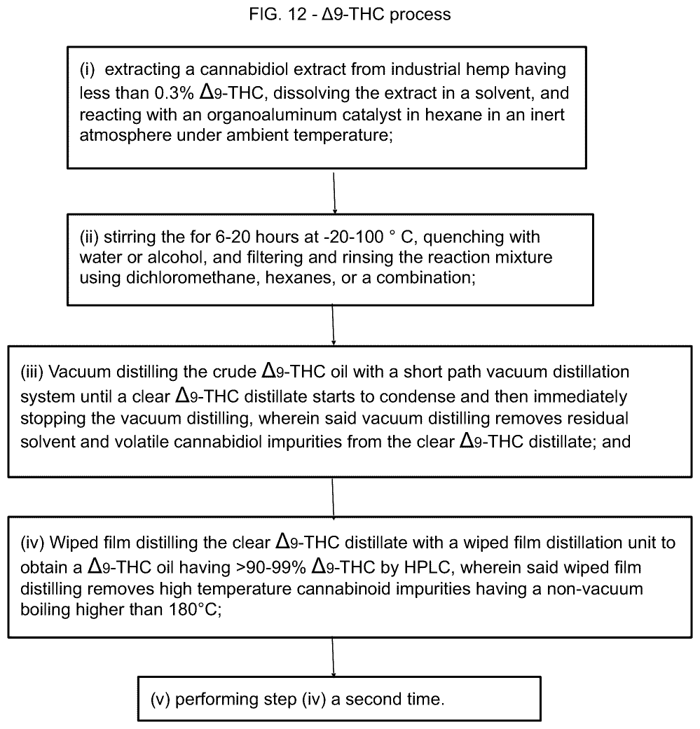

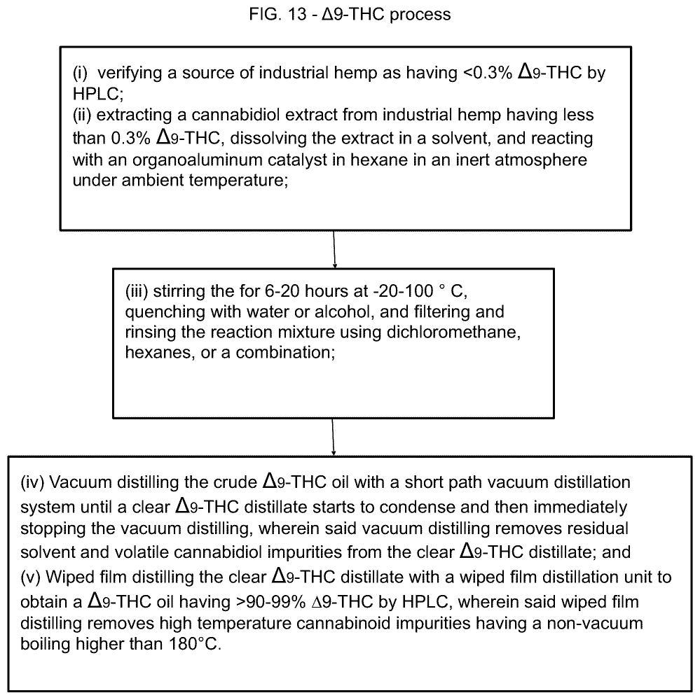

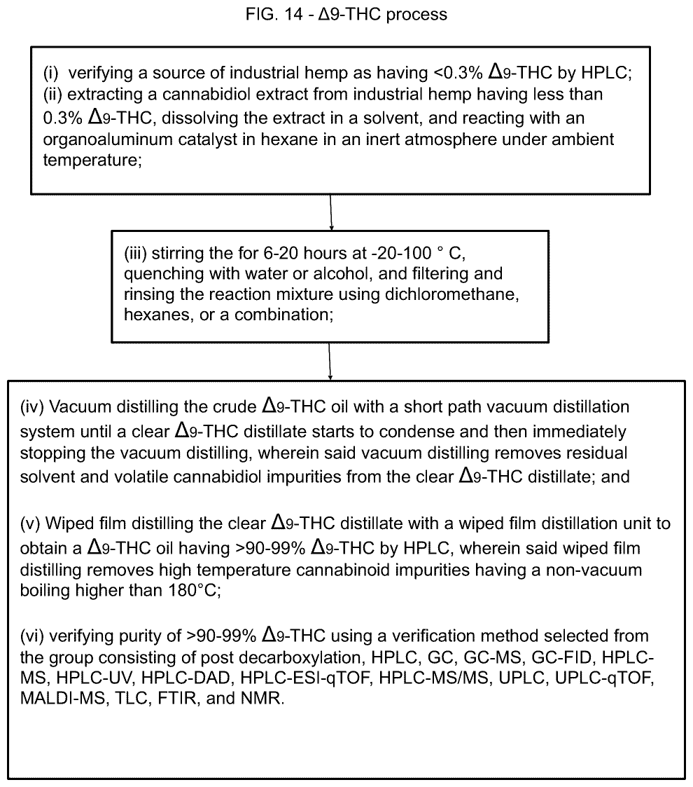

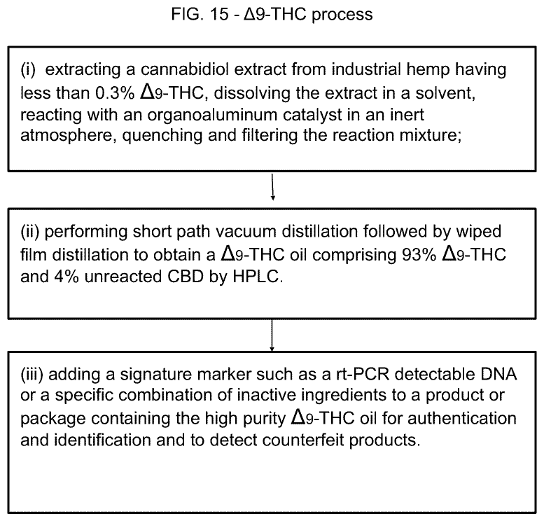

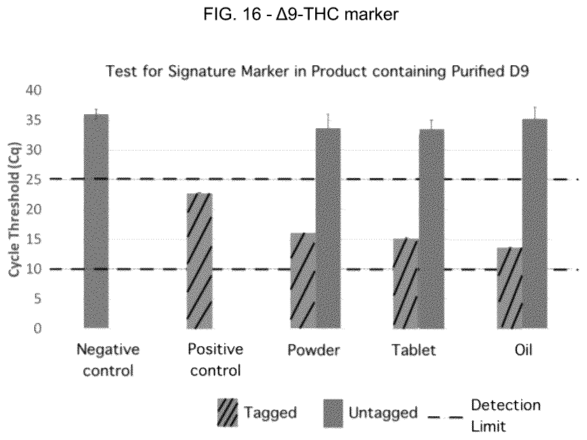

Step 3. Testing products for authenticity includes where a product containing the signature marker molecule will be positively identified in authentic products, and products that do not contain the signature marker molecule can be identified as counterfeit products.

Where samples are analyzed by rt-PCR, the resulting cycle threshold (Cq) can be compared against negative and positive controls, as shown in FIG. 8. A higher Cq value indicates more cycle time to amplify DNA tag to threshold and therefore lower quantity of DNA. Any Cq value greater than 25 is considered non-detectable. The negative control and untagged product provided a Cq>30 indicating no detection of the tag. In comparison, the tagged product samples and positive control had Cq values of 10-25 confirming the presence of the tag.

### Experimental Introduction:

Unless otherwise noted, reagents and solvents were used as received from commercial suppliers.

TLC analysis of Δ9-THC for in-process control: Analyses were obtained using MilliporeSigma TLC Silica Gel 60 F254 2.5×7.5 cm plates eluted with a 1:1 mixture of chloroform and 50% benzene and visualized in TLC chamber with UV 254 light.

Key Rf values: CBD 0.5, Δ9-THC 0.7 and Δ8-THC 0.8.

HPLC of Δ9-THC is a non-limiting preferred method for in-process control and product purity. In a specific, non-limiting example, the Δ9-THC purity was obtained using the following HPLC system. Analyses were obtained on a Shimadzu HPLC machine, Model LC 2030C-Plus using a Raptor ARC-18, 2.7 um, 150×4.6 mm column, at 35° C. column temperature, with a UV detector set at 220 nm and solvent gradient program:

TABLE
HPLC

| Time | Flow | % | % |
|---|---|---|---|
| (min) | (mL/min) | A | B |
| 0.0 | 1.6 | 30.0 | 70.0 |
| 3.0 | 1.6 | 30.0 | 70.0 |
| 7.0 | 1.6 | 15.0 | 85.0 |
| 7.01 | 1.6 | 5.0 | 95.0 |
| 8.0 | 1.6 | 5.0 | 95.0 |
| 10.0 | 1.6 | 30.0 | 70.0 |

Mobile Phase A=Water with 0.085% v/v Phosphoric acid

Mobile Phase B=Acetonitrile with 0.085% Phosphoric acid

Total Run Time: 10 min

### General Definitions

The embodiments herein and the various features and advantageous details thereof are explained more fully with reference to the non-limiting embodiments that are illustrated in the accompanying drawings and detailed description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

### EQUIVALENTS

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

The invention claimed is:

1. A process of making a self-emulsifying nano-concentrate for encapsulating a lipophilic active ingredient, comprising the steps: dissolving 95% phosphatidylcholine into 60° C. ethanol; heating the lipophilic active ingredient to 60° C. and adding it to the hot ethanol/PC solution; stirring until homogenized; dissolving Poloxamer 407 to the solution; stirring glycerin, d-limonene terpenes, cherry tart extract, and a natural flavor blend into the solution; and adding to an aqueous solution to obtain the self-emulsifying nano-concentrate having the encapsulated lipophilic active ingredient, wherein the self-emulsifying nano-concentrate is optically clear (transparent), has no lingering taste, and is shelf stable.

2. A process of making a self-emulsifying nano-concentrate for encapsulating a hydrophilic ingredient, comprising the steps: dissolving phosphatidylcholine into 60° C. ethanol; separately combining reverse osmosis water and a hydrophilic active ingredient to form an aqueous solution; adding the ethanol/PC solution to the aqueous solution to obtain the self-emulsifying nano-concentrate having the encapsulated hydrophilic active ingredient, wherein the self-emulsifying nano-concentrate is optically clear (transparent), has no lingering taste, and is shelf stable.

3. The process according to claim 1, wherein making a combined concentrate for encapsulating an active lipophilic ingredient and an active hydrophilic ingredient, comprising the steps: providing the self-emulsifying nano-concentrate having a lipophilic active ingredient that was made in claim 1 and heating to 60° C.; separately combining reverse osmosis water and a hydrophilic active ingredient to form an aqueous solution; adding the self-emulsifying nano-concentrate having a lipophilic active ingredient to the aqueous solution having the hydrophilic active ingredient to obtain a combined nano-concentrate having the lipophilic active ingredient and the hydrophilic active ingredient, wherein the combined nano-concentrate is optically clear (transparent), has no lingering taste, and is shelf stable.

4. The process according to claim 1, wherein making an ethanol-free oral formulation for encapsulating a lipophilic active ingredient, comprising the steps: providing the self-emulsifying nano-concentrate having the lipophilic active ingredient that was made in claim 1 and heating to 60° C.; loading the heated self-emulsifying nano-concentrate into a rotary evaporator; raising the temperature to 80° C. to condense and collect the ethanol, and optionally, introducing vacuum at 700 micron until no more ethanol is visible on the condenser; cooling to room temperature; pulling the vacuum down to 500 micron and holding the vacuum overnight to ensure all ethanol is removed and to obtain a film; heating the film back to 50° C.; introducing reverse osmosis water to the film and spinning on a rotary evaporator to produce invasomes as the ethanol-free oral formulation.

5. A process of making an ethanol-free topical formulation for encapsulating a lipophilic active ingredient, comprising the steps: dissolving 95% phosphatidylcholine into ethyl lactate at 60° C.; heating the lipophilic active ingredient to 60° C. and adding to the hot ethyl lactate/PC solution; adding PEG-40 Hydrogenated Castor Oil to the ethyl lactate/PC solution; adding d-limonene terpenes to the ethyl lactate/PC/PEG solution to obtain the ethanol free topical formulation having a lipophilic active ingredient.

6. A process of making a natural topical formulation for encapsulating a lipophilic active ingredient, comprising the steps: dissolving phosphatidylcholine into 60° C. ethanol; heating the lipophilic active ingredient to 60° C. and adding to the hot ethanol/PC solution; dissolving decyl glucoside (DG) into the ethanol/PC solution; adding a solution of d-limonene terpenes to the ethanol/PC/DG solution to obtain the natural topical formulation having a lipophilic active ingredient.

7. A process of making a glyceride formulation for encapsulating a lipophilic active ingredient, comprising the steps: dissolving Capmul MCM mono & di-glyceride emulsifier and Captex 355 caprylic/capric triglyceride (glyceride) into ethanol at 60° C.; heating the lipophilic ingredient to 60° C. and adding to the hot ethanol/glyceride solution; adding Poloxamer 407 to the ethanol/glyceride solution; adding glycerin, d-limonene terpenes, cherry tart extract, and natural flavor blend to obtain the glyceride formulation having the lipophilic active ingredient.

8. A process of making a sub-100 nm invasome formulation for encapsulating a lipophilic active ingredient, comprising the steps: providing the self-emulsifying nano-concentrate having the lipophilic active ingredient that was claim 1 and heating to 60° C.; adding reverse osmosis water to the self-emulsifying nano-concentrate on a spinning rotary evaporator to produce invasomes; loading the invasomes into a high pressure homogenizer; in one hour (4 cycles) applying 15,000 PSI to the solution; Once complete, changing the pressure to 25,000 PSI and running for an additional 2 hours (4 cycles), to obtain the sub-100 nm invasome formulation having the lipophilic active ingredient.

9. The process according to claim 1, wherein making a lyophilized ingestible/topical freeze dried powder, comprising the steps: providing the self-emulsifying nano-concentrate having the lipophilic active ingredient that was claim 1 and heating to 60° C.; adding Bovine Serum Albumin (BSA) to the self-emulsifying nano-concentrate; freezing the self-emulsifying nano-concentrate/BSA solution at −25° C. overnight; loading the frozen self-emulsifying nano-concentrate/BSA solution into a freeze dryer and running through at −20° C. for 12 hours at 350 microns of vacuum, to obtain the finished lyophilized ingestible/topical freeze dried powder having the lipophilic active ingredient, wherein the finished powder has a grainy consistency and is re-hydrated into nanoparticles by adding at least equal parts finished powder to reverse osmosis water.

10. A process of making a lyophilization inhalable freeze dried powder for encapsulating a lipophilic active ingredient, comprising the steps: dissolving 95% phosphatidylcholine into ethanol at 60° C.; heating the lipophilic active ingredient to 60° C. and adding to the hot ethanol/PC solution; dissolving PEG-40 Hydrogenated Castor Oil into the ethanol/PC solution; adding d-limonene terpenes and mannitol to the ethanol/PC/PEG solution; freezing the ethanol/PC/PEG solution at −25° C. overnight; loading the frozen solution into a freeze dryer and running through at −20° C. for 12 hours at 350 microns of vacuum to obtain a finished powder having the lipophilic active ingredient, wherein the finished powder has a grainy consistency and wherein the finished powder is loaded into a metered dose inhaler, dry powder inhaler, or a nebulizer for delivery.

11. The process according to any of the preceding claims 1, and 3-10 wherein the lipophilic active ingredient is selected from the group consisting of: Δ8-tetrahydrocannabinol, Δ9-tetrahydrocannabinol (THC), cannabidiol (CBD), CBD distillate, CBD isolate, cannabinol (CBN), cannabigerol (CBG), A9(11)-tetrahydrocannabinol (exo-THC), cannabichromene (CBC), tetrahydrocannabinol-C3 (THC-C3), tetrahydrocannabutol (THC-C4), and mixtures thereof.

12. The process according to any of the preceding claims 1, and 3-10 wherein the lipophilic active ingredient is selected from the group consisting of: >99% pure 48-tetrahydrocannabinol (THC) oil having less than 0.3% Δ9-THC; Δ9-tetrahydrocannabinol (THC) oil comprising over about 90% Δ9-THC, cannabidiol (CBD), CBD distillate, CBD isolate, and mixtures thereof.

13. A composition having a lipophilic active ingredient made using the process according to any of the preceding claims 1, and 3-11.

14. The composition of claim 13, wherein the lipophilic active ingredient is selected from the group consisting of: >99% pure 48-tetrahydrocannabinol (THC) oil having less than 0.3% Δ9-THC; Δ9-tetrahydrocannabinol (THC) oil comprising over about 90% Δ9-THC, cannabidiol (CBD), CBD distillate, CBD isolate, and mixtures thereof.

15. The process according to preceding claim 2 wherein the hydrophilic active ingredient is a hydrophilic prodrug of a cannabinoid, the hydrophilic prodrug group selected from the group consisting of hemisuccinate, valine hemisuccinate, organophosphate ester (O-phosphate), acetate ester (O-acetate), and morpholinylbutyrate, and wherein the cannabinoid is selected from from the group consisting of: Δ8-tetrahydrocannabinol, Δ9-tetrahydrocannabinol (THC), cannabidiol (CBD), CBD distillate, CBD isolate, cannabinol (CBN), cannabigerol (CBG), Δ9(11)-tetrahydrocannabinol (exo-THC), cannabichromene (CBC), tetrahydrocannabinol-C3 (THC-C3), tetrahydrocannabutol (THC-C4), and mixtures thereof.

16. A composition having a hydrophilic active ingredient made using the hydrophilic process of claim 2 or claim 15.

17. A composition according to preceding claim 13, 14, or 16 loaded into a capsule or beadlet for product delivery, wherein the capsule or beadlet is formulated to dissolve in stomach gastric fluid and form an in situ self-emulsifying nano-concentrate.

18. The process according to any of claims 1-10, wherein Δ8-tetrahydrocannabinol is obtained by a process, comprising:
(i) refluxing a cannabidiol extract from industrial hemp having less than 0.3% 49-THC in a mixture of toluene and p-toluenesulfonic acid monohydrate at about 70° C.-100° C. for about 120-1440 minutes to obtain a reaction mixture having less than 0.3% Δ9-THC;
(ii) adding aqueous sodium bicarbonate to neutralize the reaction mixture, adding water, and evaporating to obtain a crude Δ8-THC oil having about 73.59-99.73% Δ8-THC by HPLC and less than 0.3% Δ9-THC by HPLC.

19. The process of claim 18, wherein the p-toluenesulfonic acid monohydrate is about 0.12-0.598% (w/w) and the cannabidiol extract is about 23% (wt/wt).

20. The process of claim 18, comprising (iii) Vacuum distilling the crude Δ8-THC oil with a short path vacuum distillation system until a clear Δ8-THC distillate starts to condense and then immediately stopping the vacuum distilling, wherein said vacuum distilling removes residual solvent and volatile cannabidiol impurities from the clear Δ8-THC distillate; and (iv) Wiped film distilling the clear Δ8-THC distillate with a wiped film distillation unit to obtain a Δ8-THC oil having >99% Δ8-THC by HPLC, wherein said wiped film distilling removes high temperature cannabinoid impurities having a non-vacuum boiling higher than 180° C.

21. The process of claim 20, comprising wherein the wiped film distilling is performed twice.

22. The process of claim 18, wherein the source of cannabidiol extract is selected from the group consisting of CBD crude, CBD distillate, and CBD isolate, and wherein the mixture is refluxed at 70 C for 120 minutes, the aqueous sodium bicarbonate is 10% NaHCO₃, and the crude Δ8-THC oil is 91.68%-99.73% Δ8-THC by HPLC.

23. The process according to claim 18 wherein the mixture includes a second organic solvent selected from the group consisting of dichloromethane, dichloroethane, cyclohexane, ethanol, hexanes, heptanes, and a combination thereof, and wherein the mixture includes a second catalyst selected from the group consisting of Zinc Chloride, Hydrochloric acid, Sulfuric acid, Zinc Bromide, Boron Trifluoride, Boron Trifuluoride Diethyl Ethereate, and a combination thereof.

24. The process according to claim 18 wherein the refluxing is selected from the group consisting of a broad reflux performed for between 0.5 to about 48 hours, a medium range reflux performed for between 60 to 180 min, and a specific reflux performed for approximately 120 min., and wherein the resulting crude Δ8-THC oil is further purified using fractional, vacuum, short path, molecular, and/or wiped film distillation.

25. The method according to claim 24 wherein the dilution ratio of the Cannabinoid extract to the organic solvent is 3 to 6 on a weight basis.

26. The method according to claim 25, wherein the source Cannabidiol extract is CBD crude, CBD isolate or CBD distillate, wherein the organic solvent is toluene, wherein the catalyst is 2.6% of p-toluenesulfonic acid monohydrate, and wherein the refluxing is performed for between 60 to 180 minutes at a reaction temperature selected from the group consisting of a range between 50° C. to 100° C., a range between 60° C. to 80° C., and approximately 70° C.

27. The method according to claim 26 wherein the crude Δ8-THC having >99% purity is eluted with a second solvent or solvent mixture and separated from Δ9-THC on a Normal Phase HPLC column or a Reverse Phase HPLC column, following washing the column with the second solvent or solvent mixture, wherein the second solvent or solvent mixture is selected from toluene, ether in petroleum ether, and water-acetonitrile, wherein the eluting solvent or solvent mixture is the same as the washing solvent or solvent mixture.

28. The method according to claim 27, wherein the organic solvent consists essentially of dichloromethane, dichloroethane, ethanol, cyclohexane, hexanes, heptanes, toluene, and a combination thereof.

29. The method according to claim 28, wherein the catalyst is selected from the group consisting of Zinc Chloride or Hydrochloric acid or Sulfuric acid or Zinc Bromide or Boron Trifluoride or Boron Trifluoride Diethyl Ethereate, p-toluenesulfonic acid monohydrate, and a combination thereof.

30. The method according to claim 29, wherein the acidic reaction mixture is neutralized using a quenching agent followed by addition of purified water, the quenching agent selected from the group consisting of sodium carbonate, sodium bicarbonate, sodium sulfate, sodium thiosulfate, a 10% NaHCO₃ solution, and a combination thereof.

31. The process according to any of claims 1-10, wherein Δ8-tetrahydrocannabinol is obtained by a process, comprising:
dissolving 5 kg to 500 kg of CBD isolate with 25 to 250 liters of toluene to form a solution;
loading the solution into a reaction vessel and heating;
adding p-toluenesulfonic acid monohydrate (100 to 2 kg) to the reaction vessel and refluxing at 60° C.-80° C. for 100-150 minutes;
quenching the mixture with aqueous 10% NaHCO₃, and then adding purified water;
evaporating the mixture to collect a crude oil having greater than 90% Δ8-THC;
loading the crude oil into a short path vacuum distillation system having Raschig rings in a condensing head and heating to remove residual solvent and terpenes and obtain a clear distillate;
loading the clear distillate into a wiped film distillation unit and collecting a distilled oil having greater than 99% 48-tetrahydrocannabinol (Δ8-THC).

38. The process according to any of claims 1-10, wherein Δ9-tetrahydrocannabinol is obtained by a process, comprising:
extracting CBD distillate or isolate from industrial hemp having less than 0.3% Δ9-THC;
dissolving the CBD distillate or isolate in dichloromethane to create a homogenized mixture;
adding the homogenized mixture to a reactor vessel and adding a 10 mol % solution of organoaluminum catalyst in inert hydrocarbon solvent slowly over 30 minutes at a temperature of 18-30° C. to create a reaction mixture;
stirring the reaction mixture for approximately 6-20 hours at a temperature of −20° C. to about 70° C.;
quenching the reaction mixture with water or a C2-C4 alcohol, and stirring for 1 hour;
filtering the reaction mixture through a filter selected from diatomaceous earth, perlite, bentonite clay, celite, cellulose, or a mixture thereof, to collect a filtrate, and rinsing the filter and reaction vessel with a rinse solvent selected from dichloromethane, hexanes, or a combination of both, removing the water or a C2-C4 alcohol quench layer from the rinse, and combining the filtrate and the rinse to obtain a combined filtrate and rinse mixture;
performing a split path distillation of the combined filtrate and rinse mixture, wherein the split path distillation comprises vacuum distilling the combined filtrate and rinse mixture with a short path vacuum distillation system, wherein said vacuum distilling removes the rinse solvent and volatile cannabidiol impurities having a boiling point less than about 157° C. at about 15-20 mTorr vacuum to obtain Δ9-THC crude distillate, and then performing a vacuum wiped film distillation with a wiped film distillation unit at about 160° C. at about 15-20 mTorr vacuum to obtain a Δ9-THC oil comprising over about 90% Δ9-THC and about 4% or less of unreacted CBD, wherein said wiped film distilling removes high temperature cannabinoid impurities having a non-vacuum boiling higher than 160° C.

39. The process of claim 38,
wherein the extract from industrial hemp having less than 0.3% Δ9-THC is CBD distillate comprising at least 85% CBD and;
wherein the solvent is dichloromethane;
wherein the organoaluminum catalyst is triisobutylaluminum in inert hydrocarbon solvent (iBu3Al);
wherein quenching uses water;
wherein the filter is a diatomaceous earth filter;
wherein split path distillation comprises short path distillation first to concentrate the filtrate under reduced pressure to obtain a main portion separated from a heads portion and a tails portion, followed by wiped film distillation of the main portion; and,
wherein the Δ9-THC oil comprises 95% or greater Δ9-THC and 2% or less unreacted CBD.

40. The process of claim 39,
wherein the extract from industrial hemp having less than 0.3% Δ9-THC is CBD isolate;
wherein the solvent is dichloromethane;
wherein the organoaluminum catalyst is triisobutylaluminum (iBu3Al) in inert hydrocarbon solvent;
wherein quenching uses water;
wherein the filter is a diatomaceous earth filter;
wherein split path distillation comprises short path distillation first to concentrate the filtrate under reduced pressure to obtain a main portion separated from a heads portion and a tails portion, followed by wiped film distillation of the main portion; and,
wherein the Δ9-THC oil comprises 95% or greater Δ9-THC and 2% or less unreacted CBD.

41. The process of claim 40, wherein the CBD extract is about 85% CBD and 15% other cannabinoids, wherein the organoaluminum catalyst is 1-2 molar triisobutylaluminum in hexane, wherein the temperature of 25-30° C. to create a reaction mixture, and wherein the stirring is performed until CBD is 2-4% in the reaction mixture.

42. The process of claim 41, wherein the CBD extract is optically active chiral CBD having an R,R or trans(−) rotation.

43. The process according to claim 42, wherein the organoaluminum catalyst is selected from the group consisting of a trialkyl- or triarylaluminum, dialkyl- or diarylaluminum halide, alkylarylaluminum halide, dialkyl- or alkylaryl- or diarylaluminum alkoxide or aryloxide, dialkyl- or alkylaryl- or diarylaluminum thioalkoxide or thioarylate, dialkyl- or alkylaryl- or diarylaluminum carboxylate, alkyl- or arylaluminum dihalide, alkyl- or arylaluminum dialkoxide or diaryloxide or alkylaryloxide, alkyl- or arylaluminum dithioalkoxide or dithioarylate, alkyl- or arylaluminum dicarboxylate, aluminum trialkoxide or triaryloxide or mixed alkylaryloxide, aluminum triacylcarboxylate, and mixtures thereof.

44. The process according to claim 43, wherein the organoaluminum catalyst is a C1-C30 alkylaluminum-based catalyst.

45. The process according to claim 44, wherein the organoaluminum-based Lewis acid catalyst is ethyl aluminum dichloride, diethylaluminum chloride, diethylaluminum sesquichloride, isobutylaluminum dichloride, diisobutylaluminum chloride, or mixtures thereof.

46. The process according to claim 45, wherein the trialkylaluminum is trimethylaluminum, triethylaluminum, triisopropylaluminum, triisobutylaluminum, trioctylaluminum, or tridecylaluminum.

47. The process according to claim 46, wherein the trialkylaluminum is triisobutylaluminum (iBu3Al).

48. The process according to claim 47, wherein the trialkylaluminum is 1-2 molar triisobutylaluminum in hexane or 1 molar triisobutylaluminum in toluene.

49. The process according to claim 48, wherein the organoaluminum catalyst is in an amount of from about 0.5 mol % to about 100 mol % with respect to the homogenized mixture.

50. The process according to claim 49, wherein said organoaluminum catalyst in an amount of from about 5 mol % to about 15 mol % with respect to the homogenized mixture.

51. The process according to claim 50, comprising an additional aprotic solvent selected from toluene, hexane, heptane, xylene, dimethylformamide, dimethylsulfoxane, dichloromemethane, or a mixture thereof.

52. The process according to claim 51, wherein the solvent is dichloromethane.

53. The process according to claim 52, wherein said stirring is carried out at a temperature of from about −20° C. to about 70° C.

54. The process according to claim 53, wherein said stirring is carried out at a temperature of from about −10° C. to about 70° C.

55. The process according to claim 54, wherein said stirring is carried out at a temperature of from about 0° C. to about 40° C.

56. The process according to claim 55, wherein said treating is carried out at a temperature of from about 10° C. to about 35° C.

57. The process according to claim 56, comprising an additional purification method selected from the group consisting of chromatography, and countercurrent extraction.

58. The process according to claim 57, wherein the CBD distillate or isolate from industrial hemp having less than 0.3% Δ9-THC is combined with kief or a crude CBD extract before dissolving in dichloromethane to create a homogenized mixture.

59. The process of claim 58, wherein the homogenized mixture comprises a mixture of CBD distillate and CBD isolate.

60. The process of claim 59, wherein the homogenized mixture comprises a mixture of CBD distillate and CBD isolate and the homogenized mixture is combined with kief or a crude CBD extract.

61. A method of administering the nano-emulsion of claims 1-10 to a patient in need thereof, comprising formulating the nano-emulsion as an oral or topical composition, and delivering the oral or topical nano-emulsion composition to a patient in need thereof, wherein the patient has nausea, anxiety, stress, chronic pain, acute pain, opioid withdrawal, narcotic relapse risk, or requires an appetite stimulant.
